(19) United States
(12) Patent Application Publication
(10) Pub. No.: US 2010/0248251 A1
(43) Pub. Date: Sep. 30, 2010

(54) Tissue Rejection

(76) Inventor: Philip F. Halloran, Edmonton (CA)

Correspondence Address: FISH & RICHARDSON P.C., PO BOX 1022, MINNEAPOLIS, MN, 55440-1022, US

(21) Appl. No.: 12/797,364

(22) Filed: Jun. 9, 2010

Related U.S. Application Data
(62) Division of application No. 11/434,389, filed on May 15, 2006, now abandoned.
(60) Provisional application No. 60/681,340, filed on May 16, 2005.

(51) Int. Cl. C12Q 1/68

(52) U.S. Cl. 435/6

(57) ABSTRACT

This document relates to methods and materials involved in detecting tissue rejection (e.g., organ rejection). For example, this document relates to methods and materials involved in the early detection of kidney tissue rejection.

### CROSS-REFERENCE TO RELATED APPLICATIONS

This application is a divisional of U.S. application Ser. No. 11/434,389, filed May 15, 2006, which claims the benefit of priority from U.S. Provisional Application Ser. No. 60/681,340, filed May 16, 2005. The disclosures of the prior applications are considered part of (and are incorporated by reference in) the disclosure of this application.

### BACKGROUND

1. Technical Field

This document relates to methods and materials involved in tissue rejection (e.g., organ rejection) and detecting tissue rejection.

2. Background Information

The transplantation of tissue from one mammal to another has been used for years to save lives and to improve the quality of lives. For example, the first successful kidney transplant was performed in the mid-1950s between identical twin brothers. Since then, donors have grown to include not only close relatives but also distant relatives, friends, and total strangers. In some cases, the recipient may reject the transplanted tissue. Thus, tissue rejection is a concern for any recipient of transplanted tissue. If a doctor is able to recognize early signs of tissue rejection, anti-rejection medication often can be used to reverse tissue rejection.

### SUMMARY

This document relates to methods and materials involved in detecting tissue rejection (e.g., organ rejection). More particularly, this document relates to methods and materials involved in the early detection of tissue rejection (e.g., kidney rejection) and the assessment of a mammal's probability of rejecting tissue such as a transplanted organ. For example, this document provides nucleic acid arrays that can be used to diagnose tissue rejection in a mammal. Such arrays can allow clinicians to diagnose tissue rejection early based on a determination of the expression levels of nucleic acids that are differentially expressed in tissue being rejected as compared to control tissue not being rejected. The differential expression of such nucleic acids can be detected in tissue being rejected prior to the emergence of visually-observable, histological signs of tissue rejection. Early diagnosis of patients rejecting transplanted tissue (e.g., a kidney) can help clinicians determine appropriate treatments for those patients. For example, a clinician who diagnoses a patient as rejecting transplanted tissue can treat that patient with medication that suppresses tissue rejection (e.g., immunosuppressants).

The description provided herein is based, in part, on the discovery of nucleic acids that are differentially expressed in tissue being rejected as compared to control tissue that is not being rejected. Such nucleic acids can be nucleic acids expressed by, for example, cytotoxic T lymphocytes (CTL). The term “CTL associated transcripts” or “CATs” as used herein refers to transcripts that are expressed by activated CTL in culture at a level greater than the level of expression in normal kidney tissue. The description provided herein also is based, in part, on the discovery that the expression levels of CATs can be used to distinguish transplanted tissue that is being rejected from transplanted tissue that is not being rejected. For example, the expression levels of the nucleic acids listed in Table 4 or Table 5 can be assessed in transplanted tissue to determine whether or not that transplanted tissue is being rejected. In addition, the description provided herein is based, in part, on the discovery that the expression levels of CATs can be used to distinguish transplanted tissue that is being rejected from transplanted tissue that is not being rejected at a time point prior to the emergence of any visually-observable, histological sign of tissue rejection (e.g., tubulitis for kidney rejection).

In general, this description features a method for detecting tissue rejection. The method includes determining whether or not tissue transplanted into a mammal contains cells that express at least two of the nucleic acids listed in Table 4 or Table 5, wherein the presence of the cells indicates that the tissue is being rejected. The mammal can be a human. The tissue can be kidney tissue. The tissue can be a kidney. The method can include determining whether or not the tissue contains cells that express at least five of the nucleic acids. The method can include determining whether or not the tissue contains cells that express at least ten of the nucleic acids. The method can include determining whether or not the tissue contains cells that express at least twenty of the nucleic acids. The determining step can include measuring the level of mRNA expressed from the at least two nucleic acids. The determining step can include measuring the level of polypeptide expressed from the at least two nucleic acids. The method can include determining whether or not the tissue contains cells that express at least two of the nucleic acids at a level greater than the average level of expression exhibited in cells from control tissue that has not been transplanted.

In another embodiment, the description features a method for detecting tissue rejection. The method includes determining whether or not a sample contains cells that express at least two of the nucleic acids listed in Table 4 or Table 5, wherein the sample contains cells, was obtained from tissue that was transplanted into a mammal, and was obtained from the tissue within fifteen days of the tissue being transplanted into the mammal, and wherein the presence of the cells indicates that the tissue is being rejected. The mammal can be a human. The tissue can be kidney tissue. The tissue can be a kidney. The method can include determining whether or not the sample contains cells that express at least five of the nucleic acids. The method can include determining whether or not the sample contains cells that express at least ten of the nucleic acids. The method can include determining whether or not the sample contains cells that express at least twenty of the nucleic acids. The determining step can include measuring the level of mRNA expressed from the at least two nucleic acids. The determining step can include measuring the level of polypeptide expressed from the at least two nucleic acids. The sample can be a sample obtained from the tissue within ten days of the tissue being transplanted into the mammal. The sample can be a sample obtained from the tissue within five days of the tissue being transplanted into the mammal. The method can include determining whether or not the sample contains cells that express at least two of the nucleic acids at a level greater than the average level of expression exhibited in cells from control tissue that has not been transplanted.

In another embodiment, this description features a nucleic acid array containing at least 20 nucleic acid molecules, wherein each of the at least 20 nucleic acid molecules has a different nucleic acid sequence, and wherein at least 50 percent of the nucleic acid molecules of the array comprise a sequence from nucleic acid selected from the group consisting of the nucleic acids listed in Table 4 and Table 5. The array can contain at least 50 nucleic acid molecules, wherein each of the at least 50 nucleic acid molecules has a different nucleic acid sequence. The array can contain at least 100 nucleic acid molecules, wherein each of the at least 100 nucleic acid molecules has a different nucleic acid sequence. Each of the nucleic acid molecules that comprise a sequence from nucleic acid selected from the group can contain no more than three mismatches. At least 75 percent of the nucleic acid molecules of the array can contain a sequence from nucleic acid selected from the group. At least 95 percent of the nucleic acid molecules of the array can contain a sequence from nucleic acid selected from the group. The array can contain glass. The at least 20 nucleic acid molecules can contain a sequence present in a human.

In another embodiment, this description features a computer-readable storage medium having instructions stored thereon for causing a programmable processor to determine whether one or more nucleic acids listed in Table 4 or Table 5 are detected in a sample, wherein the sample is from a transplanted tissue. The computer-readable storage medium can further comprise instructions stored thereon for causing a programmable processor to determine whether one or more of the nucleic acids listed in Table 4 or Table 5 is expressed at a greater level in the sample than in a control sample of non-transplanted tissue.

This description also features an apparatus for determining whether a transplanted tissue is being rejected. The apparatus can include one or more collectors for obtaining signals representative of the presence of one or more nucleic acids listed in Table 4 or Table 5 in a sample from the transplanted tissue and a processor for analyzing the signals and determining whether the tissue is being rejected. The one or more collectors can be adapted to obtain further signals representative of the presence of the one or more nucleic acids in a control sample from non-transplanted tissue.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of a process for determining whether a transcript is classified as a CAT.

FIG. 2 contains photographs of the histopathology of rejecting mouse allografts using PAS staining (magnification 40×). Panel A: isograft (CBA into CBA) at day 5 with normal histology. Panel B: rejecting kidney allograft (CBA into B6) at day 5 with periarterial mononuclear interstitial infiltration. Panel C: rejecting kidney allograft at D7 (CBA into B6) with mononuclear interstitial infiltration and mild tubulitis. Panel D: kidney transplant (CBA into B6) at day 21 with heavy tubulitis.

FIG. 3 is a graph plotting the reproducibility of gene expression analysis. Gene expression values (n=22,690) from two biological replicates of pools of three kidneys rejecting in wild-type hosts at D5 (WTD5) demonstrate good reproducibility of microarray data (r=0.92).

FIG. 4 contains graphs plotting the correlation of gene expression analysis for 12 selected genes using microarrays versus real-time RT-PCR. The time course of gene expression in kidneys rejecting at day 5, 7, and 21 post transplant in selected genes (fold change versus normal kidney (NCBA)) for RT-PCR data (left) and microarrays (right).

FIG. 5 is a diagram of unsupervised hierarchical clustering of experimental groups. Unsupervised clustering of all genes, based on distance, demonstrates three main groups with a good separation between (1) isografts (ISO), (2) allografts rejecting in wild-type hosts (WT) or B cell deficient hosts (IghKO), and (3) lymphocyte cultures (MLR=mixed lymphocyte culture; CTL=CTL clone).

FIG. 6 is a graph plotting the expression level of CATs in isografts and WT allografts. CATs were absent in normal kidney, low in isografts, but highly expressed in rejecting kidneys at day 5. The expression of this set of CATs persisted throughout the rejection process.

FIG. 7 is a bar graph plotting the expression of CATs for K-means clusters in d4MLR and WT allografts. Based on their expression in a CTL clone, CATs (n=287) cluster in 5 groups. Expression in MLR and WT allografts in clusters 1-5 is shown as the percent of expression in the CTL clone. The boxplots represent the median and quartiles of expression of CATs for each time point. The CATs of cluster 1 (n=140) had low expression in MLR, but stable expression in all allografts. The CATs of cluster 2 (n=23) were more highly expressed in MLR than CTL and exhibited relatively strongly increased expression in day 5 rejecting kidneys, further increasing expression at D14. The CATs of cluster 3 (n=74) had relatively high expression in MLR versus CTL but lower expression in rejecting kidney, fluctuating somewhat among the different times while increasing between D5 and D7. The CATs of cluster 4 (n=46) had less expression in MLR than CTL, increased expression between D5 and D14, and decreased expression thereafter. The CATs of cluster 5 (n=4) were as highly expressed in rejecting grafts as in the CTL clone and MLR.

FIG. 8 is a bar graph plotting the expression of CATs for K-means clusters in kidneys rejecting in wild-type hosts and B cell deficient hosts at D7 and D21. Cluster analysis of CATs was based on expression in WT allografts (FIG. 7). Expression for each cluster is shown for WT and IghKO D7 and D21 as the percent of expression in the CTL clone. The boxplots represent the median and quartiles of expression of CATs for each time point. Expression of CATs was slightly higher in IghKO compared to WT at D7 but exhibited some attenuation in IghKO compared to their wild-type counterparts at D21.

### DETAILED DESCRIPTION

This description provides methods and materials involved in detecting tissue rejection (e.g., organ rejection). For example, this description provides methods and materials that can be used to diagnose a mammal (e.g., a human) as having transplanted tissue that is being rejected. A mammal can be diagnosed as having transplanted tissue that is being rejected if it is determined that the tissue contains cells that express one or more CATs or that express one or more of the nucleic acids listed in Table 4 or Table 5.

The methods and materials provided herein can be used to detect tissue rejection in any mammal such as a human, monkey, horse, dog, cat, cow, pig, mouse, or rat. In addition, the methods and materials provided herein can be used to detect rejection of any type of transplanted tissue including, without limitation, kidney, heart, liver, pancreas, and lung tissue. For example, the methods and materials provided herein can be used to determine whether or not a human who received a kidney transplant is rejecting that transplanted kidney.

Any type of sample containing cells can be used to determine whether or not transplanted tissue contains cells that express one or more CATs or that express one or more of the nucleic acids listed in Table 4 or Table 5. For example, biopsy (e.g., punch biopsy, aspiration biopsy, excision biopsy, needle biopsy, or shave biopsy), tissue section, lymph fluid, blood, and synovial fluid samples can be used. In some embodiments, a tissue biopsy sample can be obtained directly from the transplanted tissue. In some embodiments, a lymph fluid sample can be obtained from one or more lymph vessels that drain from the transplanted tissue. A sample can contain any type of cell including, without limitation, cytotoxic T lymphocytes, CD4⁺ T cells, B cells, peripheral blood mononuclear cells, macrophages, kidney cells, lymph node cells, or endothelial cells.

As explained herein, a CAT refers to a transcript that is expressed by activated CTL in culture at a level greater than the level of expression in normal kidney tissue. Examples of CATs include, without limitation, the nucleic acids listed in Table 4 and/or Table 5. Additional examples of CATs can be identified using the procedures described herein. For example, the procedures described in Example 1 and Example 3 can be used to identify CATs other than those listed in Tables 4 and 5.

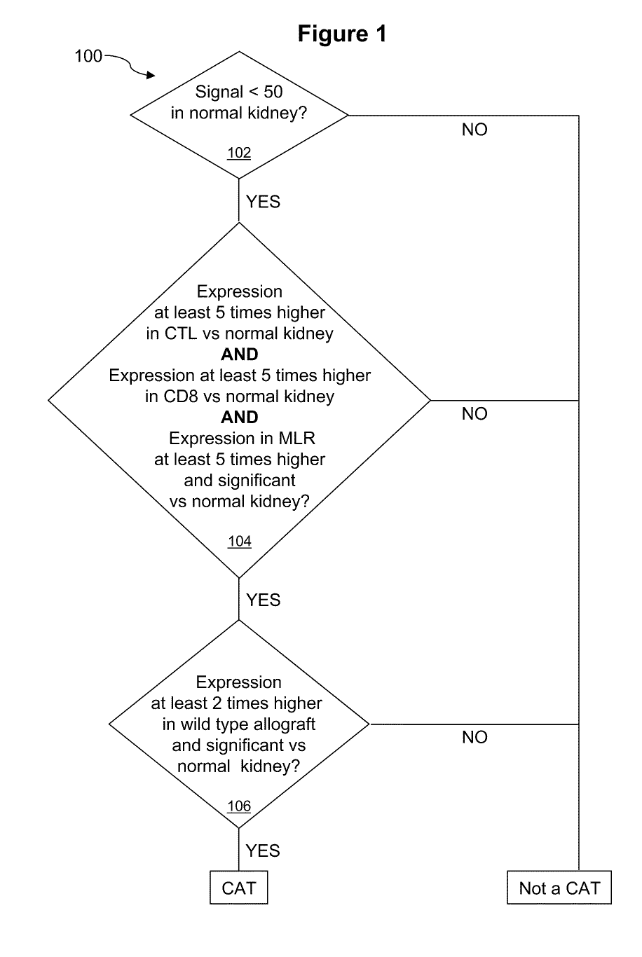

Any suitable process can be used to determine whether a particular transcript is classified as a CAT. In some embodiments, for example, a process can include determining whether a transcript is expressed in CTL and/or MLR at a level that is at least three (e.g., at least four, at least five, at least six, or at least seven) times higher than the level at which the transcript is expressed in normal kidney cells. FIG. 1 is a diagram of another embodiment of a process for determining whether a particular transcript is classified as a CAT. With reference to FIG. 1, process 100 can include step 102 for determining whether the transcript has a signal less than 50 in normal kidney (e.g., in kidney tissue from mouse strains such as CBA, B6, and Balbc), step 104 for determining whether expression of the transcript is at least five times higher in CTL as compared to expression in normal kidney, determining whether expression is at least five times higher in CD8 cells as compared to expression in normal kidney, and determining whether expression is at least five times higher in MLR and is significantly higher (p (fdr)<0.01, where “fdr” is the false discovery rate) as compared to expression in normal kidney, and step 106 for determining whether the transcript is expressed at a level that is at least two times increased in wild type allografts (CBA into B6) at day 5 and is significant (p (fdr)<0.01) as compared to expression in normal kidney. If the answer to each of these steps is “yes,” then the transcript can be classified as a CAT. If the answer to any of the steps is “no,” then the transcript is classified as not a CAT. The steps depicted in FIG. 1 can be carried out in any suitable order. Further, the steps depicted in FIG. 1 can be further divided into separate steps (e.g., step 104 can be separated into four steps, for determining (a) whether expression of the transcript is at least five times higher in CTL as compared to normal kidney, (b) whether expression is at least five times higher in CD8 cells as compared to normal kidney, (c) whether expression is at least five times higher in MLR as compared to normal kidney, and (d) whether expression in MLR is significantly higher (p (fdr)<0.01) than expression in normal kidney. Similarly, step 106 can be divided into two separate steps.

The expression of any number of CATs or nucleic acids listed in Table 4 or Table 5 can be evaluated to determine whether or not transplanted tissue is being or is likely to be rejected. For example, the expression of one or more than one (e.g., two, three, four, five, six, seven, eight, nine, ten, 15, 20, 25, 30, 40, 50, 75, 100, or more than 100) of the nucleic acids listed in Table 4 or Table 5 can be used. In some embodiments, determining that a nucleic acid listed in Table 4 or Table 5 is expressed in a sample at a detectable level can indicate that the transplanted tissue will be rejected. In some embodiments, transplanted tissue can be evaluated by determining whether or not the tissue contains cells that express a nucleic acid listed in Table 4 or Table 5 at a level that is greater than the average expression level observed in control cells obtained from tissue that has not been transplanted. Typically, a nucleic acid can be classified as being expressed at a level that is greater than the average level observed in control cells if the expression levels differ by at least 1-fold (e.g., 1.5-fold, 2-fold, 3-fold, or more than 3-fold). Control cells typically are the same type of cells as those being evaluated. In some cases, the control cells can be isolated from kidney tissue that has not been transplanted into a mammal. Any number of tissues can be used to obtain control cells. For example, control cells can be obtained from one or more tissue samples (e.g., at least 5, 6, 7, 8, 9, 10, or more tissue samples) obtained from one or more healthy mammals (e.g., at least 5, 6, 7, 8, 9, 10, or more healthy mammals).

Any suitable process can be used to determine whether a transplanted tissue is being or is likely to be rejected. In some embodiments, for example, a process can include determining whether a pre-determined number (e.g., one, two, three, four, five, six, seven, eight, nine, ten, 15, 20, 25, 30, 40, 50, 75, 100, or more than 100) of the nucleic acids listed in Table 4 or Table 5 is expressed in a sample (e.g., a sample of transplanted tissue) at a detectable level. If the number of nucleic acids that are expressed in the sample is equal to or exceeds the pre-determined number, the transplanted tissue can be predicted to be rejected. If the number of nucleic acids that are expressed in the sample is less than the pre-determined number, the transplanted tissue can be predicted to not be rejected. The steps of this process (e.g., the detection, or non-detection, of each of the nucleic acids listed in Table 4 or Table 5) can be carried out in any suitable order. In some embodiments, a process can include determining whether a pre-determined number of the nucleic acids listed in Table 4 or Table 5 is expressed in a sample at a level that is greater than the average level observed in control cells (e.g., cells obtained from tissue that has not been transplanted. If the number of nucleic acids having increased levels of expression in the sample is equal to or exceeds the pre-determined number, the transplanted tissue can be predicted to be rejected. If the number of nucleic acids having increased expression levels in the sample is less than the pre-determined number, the transplanted tissue can be predicted to not be rejected. Again, the steps of this process can be carried out in any suitable order.

Any suitable method can be used to determine whether or not a particular nucleic acid is expressed at a detectable level or at a level that is greater than the average level of expression observed in control cells. For example, expression of a particular nucleic acid can be measured by assessing mRNA expression. mRNA expression can be evaluated using, for example, northern blotting, slot blotting, quantitative reverse transcriptase polymerase chain reaction (RT-PCR), real-time RT-PCR, or chip hybridization techniques. Methods for chip hybridization assays include, without limitation, those described herein. Such methods can be used to determine simultaneously the relative expression levels of multiple mRNAs. Alternatively, expression of a particular nucleic acid can be measured by assessing polypeptide levels. For example, polypeptide levels can be measured using any method such as immuno-based assays (e.g., ELISA), western blotting, or silver staining.

The methods and materials provided herein can be used at any time following a tissue transplantation to determine whether or not the transplanted tissue is being or is likely to be rejected. For example, a sample obtained from transplanted tissue at any time following the tissue transplantation can be assessed for the presence of cells expressing a nucleic acid listed in Table 4. In some cases, a sample can be obtained from transplanted tissue 1, 2, 3, 4, 5, 6, 7, 8, or more hours after the transplanted tissue was transplanted. In some cases, a sample can be obtained from transplanted tissue one or more days (e.g., 2, 3, 4, 5, 6, 7, 8, 9, 10, 15, 20, 25, 30, 40, or more days) after the transplanted tissue was transplanted. Typically, a sample can be obtained from transplanted tissue 2 to 7 days (e.g., 5 to 7 days) after transplantation and assessed for the presence of cells expressing one or more CATs or expressing one or more nucleic acids listed in Table 4.

This description also provides nucleic acid arrays. The arrays provided herein can be two-dimensional arrays, and can contain at least 10 different nucleic acid molecules (e.g., at least 20, at least 30, at least 50, at least 100, or at least 200 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In addition, each nucleic acid molecule can have any sequence. For example, the nucleic acid molecules of the arrays provided herein can contain sequences that are present within the nucleic acids listed in Table 4. For the purpose of this document, a sequence is considered present within a nucleic acid listed in Table 4 when the sequence is present within either the coding or non-coding strand. For example, both sense and anti-sense oligonucleotides designed to human CD2 nucleic acid are considered present within CD2 nucleic acid.

Typically, at least 25% (e.g., at least 30%, at least 40%, at least 50%, at least 60%, at least 75%, at least 80%, at least 90%, at least 95%, or 100%) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid listed in Table 4. For example, an array can contain 100 nucleic acid molecules located in known positions, where each of the 100 nucleic acid molecules is 100 nucleotides in length while containing a sequence that is (1) 30 nucleotides in length, and (2) 100 percent identical, over that 30 nucleotide length, to a sequence of one of the nucleic acids listed in Table 4. A nucleic acid molecule of an array provided herein can contain a sequence present within a nucleic acid listed in Table 4, where that sequence contains one or more (e.g., one, two, three, four, or more) mismatches. Similarly, an array can contain 100 nucleic acid molecules located in known positions, where each of the 100 nucleic acid molecules is 100 nucleotides in length while containing a sequence that is (1) 30 nucleotides in length, and (2) 100 percent identical, over that 30 nucleotide length, to a sequence of one of the nucleic acids listed in Table 5. A nucleic acid molecule of an array provided herein can contain a sequence present within a nucleic acid listed in Table 5, where that sequence contains one or more (e.g., one, two, three, four, or more) mismatches.

The nucleic acid arrays provided herein can contain nucleic acid molecules attached to any suitable surface (e.g., plastic or glass). In addition, any method can be use to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

### Computer-Readable Medium and an Apparatus for Predicting Rejection

This disclosure further provides a computer-readable storage medium configured with instructions for causing a programmable processor to determine whether a transplanted tissue is being or is likely to be rejected. The determination of whether a transplanted tissue is being or will be rejected can be carried out as described herein; that is, by determining whether one or more of the nucleic acids listed in Table 4 or Table 5 is detected in a sample (e.g., a sample of the tissue), or is expressed at a level that is greater than the level of expression in a corresponding tissue that is not transplanted. The processor also can be designed to perform functions such as removing baseline noise from detection signals.

Instructions carried on a computer-readable storage medium (e.g., for detecting signals) can be implemented in a high level procedural or object oriented programming language to communicate with a computer system. Alternatively, such instructions can be implemented in assembly or machine language. The language further can be compiled or interpreted language.

The nucleic acid detection signals can be obtained using an apparatus (e.g., a chip reader) and a determination of tissue rejection can be generated using a separate processor (e.g., a computer). Alternatively, a single apparatus having a programmable processor can both obtain the detection signals and process the signals to generate a determination of whether rejection is occurring or is likely to occur. In addition, the processing step can be performed simultaneously with the step of collecting the detection signals (e.g., “real-time”).

Also provided herein, therefore, is an apparatus for determining whether a transplanted tissue is being or is likely to be rejected. An apparatus for determining whether tissue rejection will occur can include one or more collectors for obtaining signals from a sample (e.g., a sample of nucleic acids hybridized to nucleic acid probes on a substrate such as a chip) and a processor for analyzing the signals and determining whether rejection will occur. By way of example, the collectors can include collection optics for collecting signals (e.g., fluorescence) emitted from the surface of the substrate, separation optics for separating the signal from background focusing the signal, and a recorder responsive to the signal, for recording the amount of signal. The collector can obtain signals representative of the presence of one or more nucleic acids listed in Table 4 or Table 5 (e.g., in samples from transplanted and/or non-transplanted tissue). The apparatus further can generate a visual or graphical display of the signals, such as a digitized representation. The apparatus further can include a display. In some embodiments, the apparatus can be portable.

The invention will be further described in the following examples, which do not limit the scope of the invention described in the claims.

### EXAMPLES

### Example 1

### Early Diagnosis of Organ Rejection

Kidney rejection is mediated by infiltration of cytotoxic T lymphocytes (CTL) and diagnosed by histologic Banff lesions such as tubulitis. Using Affymetrix microarrays, the relationship between the evolution of pathologic lesions and the transcriptome in normal mouse kidneys, CBA isografts, CBA into C57Bl/6 allografts at days 5 to 42, and kidneys rejecting in B cell deficient hosts was evaluated. Histology was dominated by early infiltrate of mononuclear cells and slower evolution of severe tubulitis. A set of CATs was identified as having high expression in a CTL clone and day 4 mixed lymphocyte culture, while being absent in normal kidney. This set of CATs was fully expressed in rejecting kidneys at day 5, representing about 14 to 20 percent of the transcriptome of rejecting kidney. The expression persisted through day 42. Lack of mature B cells had little effect on expression of the set of CATs. In addition, expression of the identified set of CATs was established before diagnostic Banff lesions were observed and remained consistent through day 42 despite massive alterations in the pathology. Thus, the expression of the identified set of CATs in rejecting organs indicates the state of effector T cell infiltration, and can establish the diagnosis of T cell mediated rejection earlier and more securely than pathologic criteria.

Materials and Methods

Mice

Male CBA/J (CBA), C57Bl/6 (B6), B6.129P2-Igh-J^{tm1Cgn}(Igh-j), and B6.129S2-Igh-6^{tm1,Cgn}(Igh-6) mice were obtained from Jackson Laboratory (Bar Harbor, Me.) and maintained in the Health Sciences Laboratory Animal Services at the University of Alberta. All maintenance and experiments conformed to approved animal care protocols. CBA (H-2K, I-A^{k}) into C57Bl/6 (B6; H-2K^{b}D^{b}, I-A^{b}) mice strain combinations were studied across full MHC and non-MHC disparities. To ensure robust findings, two different types of IghKO mice, which were previously shown to have similar phenotypes as hosts for allografts (Jabs et al., Am. J. Transplant, 3(12):1501-1509 (2003)), were used.

Renal Transplantation

Donor mice of 9-11 weeks of age were anaesthetized, and the right kidney was removed through a midline abdominal incision and preserved in cold lactate Ringer's solution. Host mice were similarly anaesthetized, and the right native kidney excised. The donor kidney was anastomosed heterotopically to the aorta, inferior vena cava, and bladder on the right side, without removing the host's left kidney (non life-supporting kidney transplantation). Recovered mice were killed at day 5, 7, 14, 21, or 42 post-transplant, following anaesthesia and cervical dislocation. Kidneys were removed, snap frozen in liquid nitrogen, and stored at −70° C. No mice received immunosuppressive therapy. Kidneys with technical complications or infection at the time of harvesting were removed from the study.

Mixed Leukocyte Reaction (MLR)

CTL effectors were generated by co-culturing C57BL/61 responder splenocytes with mitomycin C-treated (5 μg/mL, Sigma Chemicals, St. Louis, Mo.) CBA splenocytes in complete RPMI 1640 medium (10% FCS, 1% antibiotic-antimycotic; Life Technologies, Grand Island, N.Y.), 1% nonessential amino acids, 1% sodium pyruvate (Flow Laboratories, McLean, Va.), and 50 μM β-ME at a concentration of 3×10⁶ cells/mL. Cultures were kept at 37° C., 5% CO₂ in 25 cm² cell culture flasks standing upright for 4 days. Cytolytic activity was confirmed by a ⁵¹Cr release assay.

CTL Culture

A CTL clone, C57/B6 anti C3H, was generated by co-culturing C57Bl/6 splenocytes with irradiated (2500 rads) C3H splenocytes at a 1:1 ratio for 3 days in RPMI 1640 medium (same composition as for the 4-day MLR). CTLs were purified using Ficoll gradient and cultured for another 4 days. Re-stimulation was performed at a 1:14 ratio for 3 days. After purification, cells were used for RNA extraction. Cytolytic activity was confirmed by a ⁵¹Cr release assay.

RNA Preparation

Total RNA was extracted from individual kidneys by the guanidinium-caesium chloride method (transplants) or by Trizol extraction (4-day MLR and CTL cultures), and RNA yields were measured by UV absorbance. Quality was assessed by the absorbance ratio, by agarose gel electrophoresis, and, in select samples, by Affymetrix T3 Test arrays (Affymetrix, Santa Clara, Calif.). For microarray analysis, equal amounts of RNA from 3 mice (20-25 μg each) were pooled and purified using the RNeasy Mini Kit (Quiagen, Ont. Canada). dsDNA and cRNA synthesis, hybridization to MOE 430A oligonucleotide arrays (Affymetrix), washing, and staining were carried out according to the manufacturer's manual. See, e.g., Affymetrix Technical Manual, 2003 version downloaded from Affymetrix's website.

Real-Time RT-PCR

To confirm the microarray results, expression of selected genes was assessed by TaqMan real-time RT-PCR. Two micrograms of RNA were transcribed using M-MLV reverse transcriptase and random primers. All TaqMan probe/primer combinations were designed using Primer Express software version 1.5 or purchased as Assay on demand (PE Applied Biosystems). cDNA was amplified in a multiplex system using murine hypoxanthine phosphoribosyltransferase (HPRT) cDNA as the control. Quantification of gene expression was performed utilizing the ABI prism 7700 Sequence Detection System (PE Applied Biosystems) as described elsewhere (Heid et al., Genome Research, 6(10):986-994 (1996)). Fold change over control kidney was determined using the ΔCt or ΔΔCt methods as described by the manufacturer.

Sample Designation and Analysis

Normal control kidneys were from CBA mice (NCBA). Allografts rejecting in wild-type hosts (B6) at day 5, 7, 14, 21, and 42 post transplant were designated WT D5, WT D7, WT D14, WT D21, and WT D42, respectively. Corresponding isografts were designated Iso D5, Iso D7, and Iso D21. Allografts rejecting in mature B cell deficient B6 hosts studied at days 7 and 21 were designated IghKO D7 and IghKO D21. Mixed leukocyte reaction, day 4, was designated as d4MLR and CTL clone, day 4, was designated as CTL. Two biological replicates (each consisting of RNA pooled from 3 mice) were tested in the following groups: WT D7, WT D14, WT D21, WT D42, Iso D7, and IghKO D7. Biological triplicates were analyzed in NCBA, WT D5, IghKO D21 (2 arrays with RNA pooled from 3 Igh-6 hosts, and 1 array with RNA pooled from 3 Igh-j hosts), and a single analysis was done in Iso D5, Iso D21, d4MLR, and CTL. Before processing for mRNA studies, every kidney was examined histologically to exclude kidneys with infection or surgical complication (global early infarction).

Initial data analysis was performed using Microarray Suite Expression Analysis 5.0 software (Affymetrix). Software default conditions were used to flag transcripts as present, marginal, or absent and to calculate the absolute signal strength. Total fluorescence for each array was globally scaled to a target value of 500. GeneSpring™ software (Version 6.1, Silicon Genetics, CA, USA) was used for further analyses. Following data importation, intensity values below 20 were adjusted to a value of 20, a per chip normalization was performed to the 50^{th} percentile, and a per gene normalization was performed using NCBA or CTL as control samples. Replicate samples were expressed as mean normalized value for further analysis. For unsupervised hierarchical cluster analysis, similarity measurements were based on distance and visualized by a tree diagram (Eisen et al., Proc. Natl. Acad. Sci., 95(25):14863-14868 (1998)). CATs were defined as CTL associated transcripts having a signal that was increased at least five-fold in CTL and MLR culture compared to the signal in normal kidney (significant by ANOVA; p<0.05), and that were “absent” (by Affymetrix GCOS software default conditions) in normal CBA kidney.

A second, more refined algorithm, used RMA (robust multichip analysis). In this process, CATs were identified based on (1) a signal less than 50 in normal kidneys in all three strains (CBA, B6, and Balb/c), (2) a signal at least 5 times higher in CTL, MLR, and CD8 as compared to normal kidneys, significantly higher (p(fdr)<0.01) in MLR vs. normal kidney, and at least 2 times higher in wild type allografts (CBA into B6) at day 5 and significant (p(fdr)<0.01) compared to normal kidney.

CATs were analyzed using a K-means cluster algorithm based on expression data normalized to the CTL clone.

Results

Pathological Lesions in Rejecting Kidneys

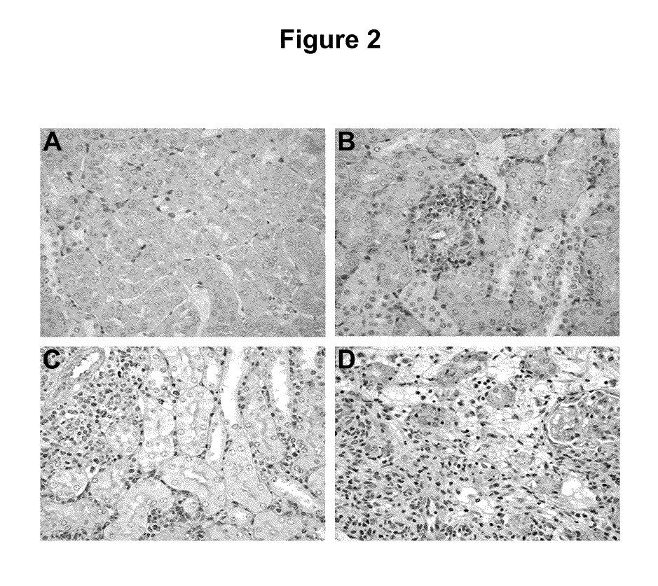

Histology of CBA kidney allografts in B6 hosts has been described elsewhere (Jabs et al., Am. J. Transplant., 3(12):1501-1509 (2003) and Halloran et al., Am. J. Transplant., 4(5):705-712 (2004)). Isografts at 5 (FIG. 2, panel A), 7, and 21 days post transplant appeared normal with no inflammation or acute tubular necrosis. Allografts exhibited an interstitial mononuclear infiltrate at day 5, which increased at day 7, and stabilized or regressed by day 21 (FIG. 2, panels B, C, and D, respectively). Tubulitis was absent at day 5, mild at day 7, and severe at days 14, 21, and 42. By immunostaining, the infiltrate in kidney allografts at days 5, 7, and 21 was comprised of 40-60 percent CD3⁺ T cells (mostly CD8⁺) and 35-50 percent CD68⁺ macrophages, with late appearance of about 5 percent CD19⁺ B cells at day 21. Hosts deficient in mature B cells (Igh6KO or IghJKO) exhibited similar infiltrate and tubulitis but less necrosis and hemorrhage at day 21 (Jabs et al., Am. J. Transplant., 3(12):1501-1509 (2003)), and 19 percent lower kidney weight (260±58 mg, n=8 versus 319±70 mg, n=6 in wild type hosts). Details of the histology of individual mice are found in Table 1 with the abbreviations being as follows: wt: weight; Tx: transplant; Nec: necrosis; PTC: peritubular capillary congestion; Glom: glomerulitis; Tub: tubulitis; Inf: interstitial infiltrate; Art: arteritis; AT: arterial thrombosis; Ven: venulitis; VT: venous thrombosis; NCBA: normal CBA kidney; iso: isograft; WT: wild-type allograft.

TABLE 1
Histology for individual mice.

| | | | | Mouse | Donor | Host | Tx | | | | | | | | | | | |
|---|---|---|---|---|---|---|---|---|---|---|---|---|---|---|---|---|---|---|
| | Donor | Host | day | ID | wt | wt | wt | Nec | PTC | Glom | Tub | Inf | Art | AT | Ven | VT | Ed | Cast |
| NCBA | CBA | — | 0 | 695 | | 24 | 170 | — | — | — | — | — | — | — | — | — | — | — |
| | | | | 627 | | | | — | — | — | — | — | — | — | — | — | — | — |
| | | | | 628 | | | | — | — | — | — | — | — | — | — | — | — | — |
| | | | | 696 | | 25 | 165 | — | — | — | — | — | — | — | — | — | — | — |
| | | | | 661 | | | | — | — | — | — | — | — | — | — | — | — | — |
| | | | | 662 | | | | — | — | — | — | — | — | — | — | — | — | — |
| | | | | 752 | | 28 | 209 | — | — | — | — | — | — | — | — | — | — | — |
| | | | | 755 | | 20 | 133 | — | — | — | — | — | — | — | — | — | — | — |
| | | | | 756 | | 20 | 132 | — | — | — | — | — | — | — | — | — | — | — |
| Iso | CBA | CBA | 5 | 727 | 34 | 28 | 249 | 0 | 0 | 1 | 0 | 0 | 0 | 0 | 2 | 0 | 0 | 0 |
| | | | | 728 | 27 | 26 | 226 | 0 | 0 | 0 | 0 | 0 | 0 | 0 | 1 | 0 | 0 | 0 |
| | | | | 740 | 29 | 28 | 242 | 0 | 0 | 0 | 0 | 0 | 0 | 0 | 0 | 0 | 0 | 0 |
| | | | 7 | 520 | 25 | 23 | 207 | 0 | 0 | 1 | 0 | 5 | 0 | 0 | 0 | 0 | 0 | 0 |
| | | | | 525 | 25 | 27 | 298 | 0 | 0 | 1 | 0 | 0 | 0 | 0 | 0 | 0 | 0 | 0 |
| | | | | 528 | 24 | 25 | 229 | 0 | 10 | 1 | 0 | 0 | 1 | 0 | 1 | 0 | 0 | 0 |
| | | | | 751 | 27 | 26 | 193 | 0 | 5 | 1 | 0 | 0 | 0 | 0 | 0 | 0 | 0 | 0 |
| | | | | 744 | 28 | 24 | 231 | 0 | 0 | 1 | 0 | 0 | 0 | 0 | 2 | 0 | 0 | 0 |
| | | | | 745 | 25 | 27 | 192 | 0 | 0 | 1 | 0 | 0 | 0 | 0 | 0 | 0 | 0 | 0 |
| | | | 21 | 513 | 27 | 27 | 193 | 0 | 0 | 1 | 0 | 0 | 0 | 0 | 1 | 0 | 0 | 0 |
| | | | | 518 | 34 | 25 | 182 | 0 | 10 | 1 | 0 | 5 | 1 | 0 | 1 | 0 | 0 | 0 |
| | | | | 531 | 26 | 27 | 204 | 0 | 0 | 1 | 0 | 0 | 0 | 0 | 0 | 0 | 0 | 0 |
| WT | CBA | B6 | 5 | 495 | 30 | 26 | 253 | 0 | 0 | 1 | 10 | 30 | 0 | 0 | 0 | 0 | 0 | 0 |
| | | | | 496 | 29 | 26 | 303 | 0 | 0 | 1 | 10 | 40 | 0 | 0 | 4 | 0 | 0 | 0 |
| | | | | 499 | 33 | 23 | 279 | 0 | 0 | 0 | 10 | 20 | 0 | 0 | 4 | 0 | 0 | 0 |
| | | | | 510 | 20 | 28 | 215 | 0 | 5 | 1 | 10 | 10 | 0 | 0 | 1 | 0 | 0 | 0 |
| | | | | 511 | 23 | 26 | 309 | 0 | 0 | 1 | 20 | 50 | 3 | 0 | 1 | 0 | 0 | 0 |
| | | | | 694 | 25 | 20 | 168 | 15 | 0 | 2 | 40 | 50 | 0 | 0 | 1 | 0 | 0 | 0 |
| | | | | 831 | | 24 | 299 | 0 | 0 | 1 | 10 | 20 | 0 | 0 | 4 | 0 | 0 | 0 |
| | | | | 874 | 30 | 24 | 270 | 0 | 0 | 1 | 15 | 20 | 0 | 0 | 2 | 0 | 0 | 0 |
| | | | 7 | 447 | | 31 | 423 | 0 | 0 | 2 | 20 | 40 | 0 | 0 | 5 | 0 | 0 | 0 |
| | | | | 455 | | 26 | 239 | 0 | 0 | 2 | 20 | 40 | 0 | 0 | 6 | 0 | 0 | 0 |
| | | | | 471 | | 26 | 338 | 0 | 0 | 2 | 20 | 60 | 0 | 0 | 8 | 0 | 0 | 0 |
| | | | | 350 | | 27 | 290 | 0 | 0 | 2 | 15 | 40 | 0 | 0 | 2 | 0 | 0 | 0 |
| | | | | 351 | | 28 | 267 | 0 | 0 | 2 | 10 | 30 | 0 | 0 | 3 | 0 | 0 | 0 |
| | | | | 352 | | 28 | 299 | 0 | 0 | 2 | 10 | 40 | 0 | 0 | 0 | 0 | 0 | 0 |
| | | | 14 | 404 | 24 | 27 | 349 | 0 | 10 | 3 | 50 | 60 | 2 | 0 | 3 | 0 | 10 | 5 |
| | | | | 405 | 23 | 27 | 389 | 0 | 15 | 3 | 50 | 40 | 4 | 1 | 3 | 0 | 15 | 5 |
| | | | | 406 | 24 | 28 | 228 | 0 | 15 | 3 | 60 | 50 | 3 | 2 | 2 | 0 | 10 | 5 |
| | | | | 403 | 24 | 25 | 321 | 0 | 10 | 3 | 50 | 60 | 1 | 1 | 4 | 0 | 0 | 0 |
| | | | | 787 | 26 | 27 | 347 | 0 | 0 | 1 | 30 | 50 | 0 | 0 | 2 | 0 | 0 | 0 |
| | | | | 859 | 27 | 25 | 358 | 5 | 0 | 2 | 50 | 30 | 0 | 0 | 2 | 0 | 0 | 0 |
| | | | 21 | 470 | | 26 | 371 | 5 | 0 | 3 | 50 | 60 | 1 | 0 | 2 | 0 | 0 | 0 |
| | | | | 346 | | 30 | 363 | 5 | 10 | 3 | 40 | 50 | 0 | 0 | 1 | 0 | 20 | 0 |
| | | | | 456 | | 32 | 398 | 10 | 0 | 3 | 60 | 70 | 1 | 0 | 3 | 0 | 20 | 10 |
| | | | | 436 | | 27 | 297 | 0 | 0 | 3 | 80 | 50 | 3 | 0 | 2 | 0 | 0 | 0 |
| | | | | 438 | | 28 | 264 | 5 | 0 | 3 | 70 | 50 | 4 | 0 | 3 | 0 | 10 | 10 |
| | | | | 445 | | 26 | 219 | 10 | 0 | 3 | 60 | 60 | 1 | 0 | 3 | 0 | 0 | 10 |
| | | | 42 | 287 | | 25 | 285 | 0 | 0 | 1 | 40 | 40 | 0 | 0 | 2 | 1 | 0 | 0 |
| | | | | 288 | | 29 | 224 | 0 | 0 | 2 | 70 | 75 | 0 | 0 | 0 | 0 | 0 | 0 |
| | | | | 566 | | 25 | 348 | 30 | 0 | 2 | 80 | 60 | 2 | 1 | 0 | 0 | 80 | 0 |
| | | | | 289 | | 29 | 627 | 75 | 0 | 2 | 30 | 30 | 0 | 2 | 1 | 0 | 75 | 0 |
| | | | | 291 | | 27 | 499 | 20 | 40 | 2 | 80 | 70 | 1 | 1 | 2 | 0 | 40 | 0 |
| | | | | 297 | | 29 | 392 | 50 | 60 | 2 | 80 | 70 | 2 | 2 | 3 | 0 | 70 | 0 |
| IghKO | CBA | Igh-6 | 7 | 116 | | 25 | 294 | 0 | 0 | 1 | 50 | 60 | 0 | 0 | 0 | 0 | 0 | 0 |
| | | | | 265 | | 22 | 330 | 20 | 0 | 1 | 30 | 70 | 5 | 1 | 5 | 0 | 0 | 5 |
| | | | | 274 | | 17 | 160 | 0 | 0 | 1 | 40 | 50 | 0 | 0 | 2 | 0 | 0 | 0 |
| | | | | 275 | | 21 | 232 | 0 | 0 | 1 | 30 | 60 | 3 | 0 | 4 | 0 | 0 | 0 |
| | | | | 276 | | 19 | 262 | 5 | 0 | 1 | 30 | 60 | 2 | 0 | 3 | 0 | 0 | 0 |
| | | | | 277 | | 20 | 286 | 0 | 0 | 1 | 30 | 70 | 0 | 0 | 3 | 0 | 0 | 0 |
| | | Igh-6 | 21 | 155 | | 28 | 283 | 0 | 0 | 1 | 30 | 0 | 0 | 0 | 0 | 0 | 0 | 0 |
| | | | | 244 | | 25 | 223 | 0 | 0 | 1 | 20 | 20 | 0 | 0 | 1 | 0 | 0 | 0 |
| | | | | 259 | | 24 | 214 | 0 | 0 | 0 | 20 | 20 | 1 | 0 | 0 | 0 | 0 | 0 |
| | | | | 156 | | 28 | 208 | 10 | 10 | 3 | 75 | 75 | 0 | 0 | 5 | 0 | 0 | 0 |
| | | | | 264 | | 25 | 216 | 0 | 10 | 2 | 75 | 75 | 1 | 1 | 0 | 0 | 0 | 0 |
| | | Igh-J | | 490 | | 21 | 373 | 0 | 0 | 1 | 5 | 30 | 0 | 0 | 2 | 0 | 5 | 0 |
| | | | | 491 | | 26 | 260 | 0 | 0 | 1 | 20 | 30 | 1 | 0 | 2 | 0 | 0 | 0 |
| | | | | 492 | | 30 | 306 | 0 | 5 | 1 | 30 | 20 | 3 | 0 | 2 | 0 | 0 | 0 |

Affymetrix Microarray Analysis and Validation

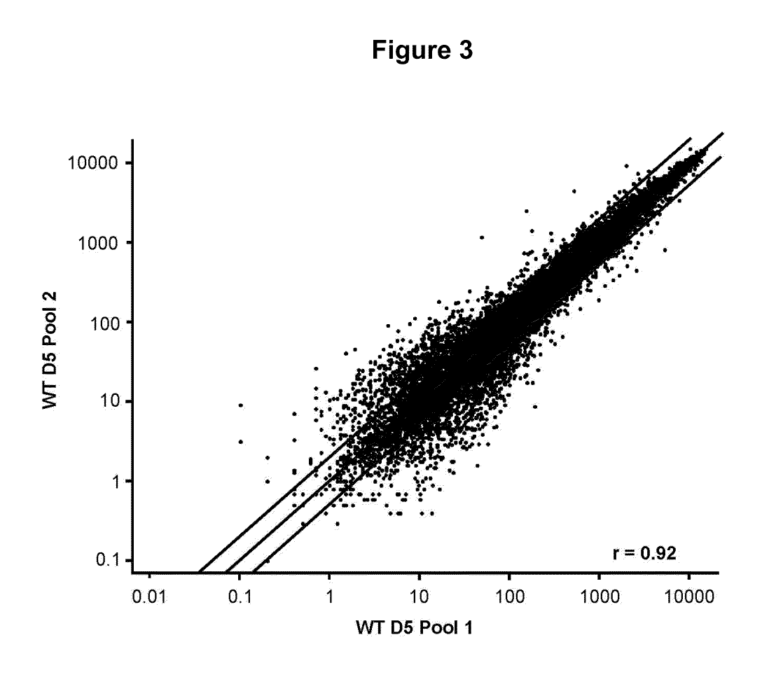

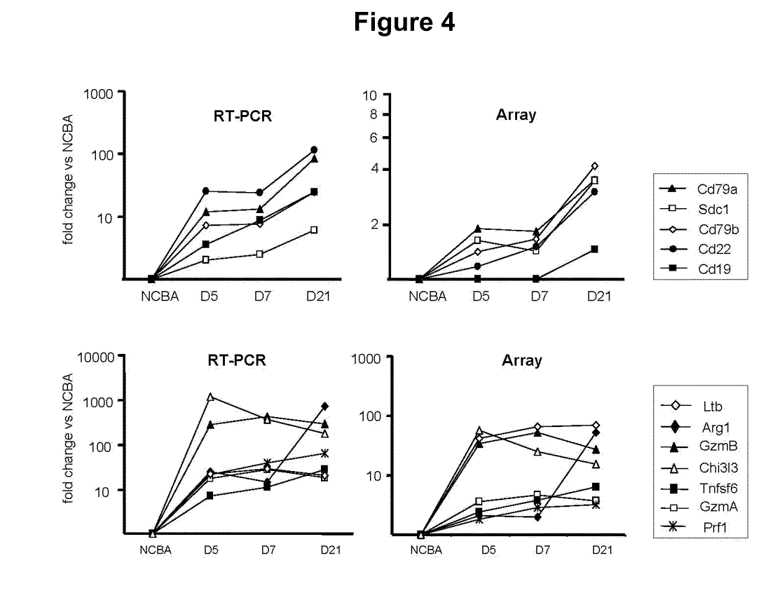

The global gene expression correlated well in biological replicates from two independent pools of three kidneys (NCBA: r=0.96; Iso D7: r=0.96; WT D5: r=0.92; WT D7: r=0.96; WT D14: r=0.98; WT D21: r=0.86; WT D42: r=0.90). The results for WT D5 transplants are presented in FIG. 3. Correlation between the d4MLR and a CTL clone was r=0.82. Microarray results were compared with real-time RT-PCR for a set of 35 genes encoding cytokines, chemokines, CD markers, and other factors involved in inflammation and cytolysis. Results from twelve selected genes are presented in FIG. 4. RT-PCR results revealed a 10 fold higher increase in gene expression when compared to the results obtained from microarrays, but the patterns of gene expression were similar for microarray and RT-PCR (r=0.87).

Hierarchical Clustering of the Global Gene Expression in Rejecting Kidneys, Isografts, CTL, and d4 MLR

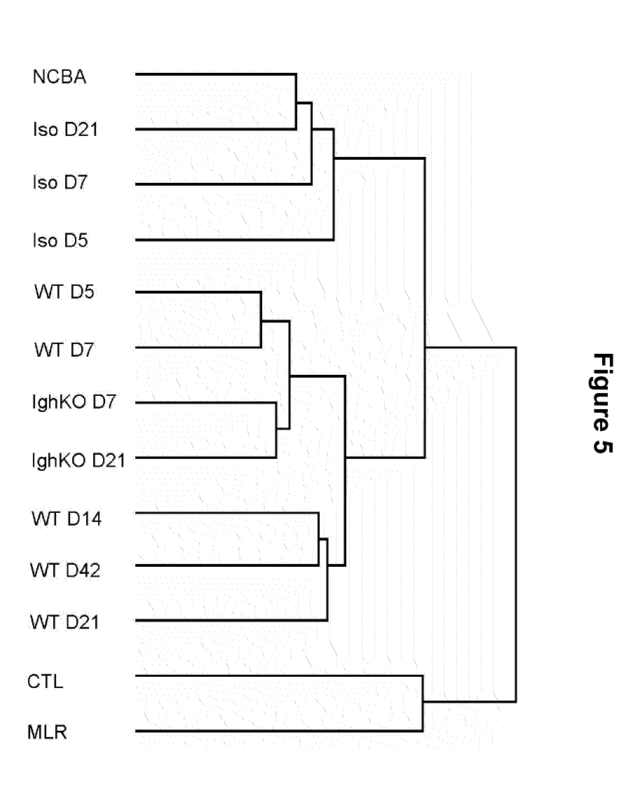

Unsupervised hierarchical cluster analysis was used to compare overall gene expression between control kidneys, isografts, allografts rejecting in WT and IghKO hosts, d4MLR, and the allostimulated CTL clone. The resulting dendrogram (FIG. 5) revealed that the transcriptomes cluster into three groups. One group included normal kidneys and isografts at days 5, 7, and 21, with Iso D21 being more similar to NCBA than Iso D5 or Iso D7. The allografts clustered in a second group, with WT D5, WT D7, IghKO D7, and IghKO D21 in one sub-cluster and WT D14, WT D21, and WT D42 in a second sub-cluster. d4MLR and CTL formed a distinct third cluster.

CD Antigen Transcript Expression

Expression of CD gene transcripts as a reflection of cellular infiltration was analyzed. Transcripts were selected by searching a master table for “CD antigen.” Genes having an expression level that was increased greater than two fold at least at one time point during rejection in allografts were chosen and compared to other samples.

The expression of thirty-three CD transcripts was increased at least two fold in wild-type allografts as compared to the expression levels observed in NCBA kidney (Table 2). Twenty-one of these had high expression in d4MLR and CTL (increased more than 5 fold as compared to NCBA). High expression of these transcripts in rejecting kidney is consistent with CTL infiltration at D5, which increases at D7 and stabilizes thereafter. CD2f10 and CD 14 were increased in rejecting allografts with no expression in d4MLR or CTL, suggesting that they represent infiltrating activated macrophages, which are poorly represented in d4MLR and absent in CTL. The relatively high CD68 expression in all rejecting grafts supports this view. The B cell specific transcripts CD79a and CD79b appeared late in rejection at days 14, 21, and 42 in wild-type but not in IghKO hosts, consistent with late recruitment of antibody-producing cells to the graft. The analysis of CD transcripts is consistent with an early and sustained CTL/macrophage infiltrate in wild-type and IghKO hosts, and with late B cell infiltration in wild-type hosts.

TABLE 2
Changes in CD antigen transcripts in isografts and kidneys rejecting in wild-type
hosts and in B cell deficient hosts.

| | | | | | | | | | | IghKO | | | |
|---|---|---|---|---|---|---|---|---|---|---|---|---|---|
| | | | | | | | | | | Allografts | | Lympho- | |
| | NCBA | Isografts | | | WT Allografts | | | | | Fold | | cytes | |
| | Signal | Fold Change | | | Fold Change | | | | | Change | | Fold Change | |
| Symbol | NCBA | D5 | D7 | D21 | D5 | D7 | D14 | D21 | D42 | D7 | D21 | CTL | MLR |
| Cd1d1 | 48 | — | — | — | — | — | 5.2 | 3.1 | 4.1 | 2.6 | 2.7 | 9.4 | 5.3 |
| Cd2 | 15 | — | — | — | 10.4 | 13.6 | 15.8 | 12.7 | 9.3 | 10.2 | 9.9 | 269.5 | 166.0 |
| Cd2f10- | 112 | — | — | — | 4.2 | 5.5 | 10.8 | 7.9 | 7.4 | 3.9 | 4.8 | — | — |
| pending | | | | | | | | | | | | | |
| Cd3d | 8 | — | — | — | 42.1 | 59.0 | 60.8 | 48.9 | 31.8 | 66.9 | 38.2 | 812.4 | 910.7 |
| Cd3e | 60 | — | — | — | 9.3 | 16.3 | 11.2 | 16.1 | 6.3 | 19.9 | 13.8 | 64.8 | 81.1 |
| Cd3g | 43 | — | — | — | 22.9 | 35.9 | 41.4 | 42.4 | 23.4 | 37.6 | 28.0 | 252.4 | 174.9 |
| Cd3z | 39 | — | — | — | 6.9 | 9.1 | 8.1 | 8.6 | 4.3 | 9.5 | 6.6 | 54.8 | 64.5 |
| Cd5 | 112 | — | — | — | 2.9 | 4.1 | 2.4 | 3.2 | — | 3.8 | 2.7 | 14.6 | 17.1 |
| Cd6 | 54 | — | — | — | 6.8 | 6.6 | — | 7.2 | — | 7.2 | 6.9 | 18.4 | 16.2 |
| Cd7 | 24 | — | — | — | — | — | — | — | — | — | 9.6 | — | — |
| Cd8a | 97 | — | — | — | 9.3 | 18.8 | 17.6 | 11.6 | 8.8 | 27.9 | 10.9 | 39.7 | 32.8 |
| Cd8b | 22 | — | — | — | 26.9 | 39.6 | 50.3 | 40.0 | 24.8 | 47.1 | 29.1 | 251.6 | 111.8 |
| Cd14 | 424 | — | 3.6 | — | 7.3 | 2.8 | 5.4 | 4.2 | 4.6 | 5.5 | 3.1 | — | — |
| Cd22 | 153 | — | — | — | — | — | 2.4 | 3.1 | 3.2 | — | 2.6 | — | 14.4 |
| Cd28 | 41 | — | — | — | 8.6 | 8.1 | 10.7 | 5.2 | 6.1 | 7.9 | 5.1 | 76.2 | 45.8 |
| Cd38 | 343 | — | — | — | — | — | 3.1 | 2.3 | 2.9 | 3.1 | 2.0 | — | 3.2 |
| Cd44 | 65 | 2.1 | 3.7 | — | 9.8 | 14.3 | 29.6 | 28.9 | 25.0 | 15.6 | 16.8 | 43.0 | 25.6 |
| Cd47 | 990 | — | — | — | 3.2 | 2.9 | 4.0 | 3.7 | 2.9 | 3.8 | 3.1 | 13.3 | 9.5 |
| Cd48 | 20 | — | 4.0 | — | 23.6 | 29.6 | 45.8 | 31.2 | 33.5 | 32.1 | 22.3 | 269.6 | 63.1 |
| Cd52 | 287 | — | 2.1 | — | 15.1 | 19.1 | 30.6 | 19.8 | 19.7 | 21.6 | 15.3 | 71.0 | 58.8 |
| Cd53 | 134 | — | 2.8 | — | 11.4 | 17.3 | 22.6 | 18.2 | 19.6 | 18.0 | 13.0 | 73.9 | 71.7 |
| Cd68 | 161 | — | — | — | 4.8 | 6.7 | 13.4 | 10.6 | 18.3 | 9.8 | 8.9 | 2.5 | 2.7 |
| Cd72 | 41 | — | — | — | 7.8 | 13.4 | 27.7 | 14.9 | 20.2 | 9.4 | 14.4 | 13.6 | 30.9 |
| Cd79a | 85 | — | — | — | — | — | 2.0 | 2.9 | 2.7 | — | — | — | 35.6 |
| Cd79b | 67 | — | — | — | — | — | 3.0 | 3.8 | 3.9 | — | — | — | 35.6 |
| Cd80 | 54 | — | — | — | — | 2.0 | 3.0 | 2.3 | 2.4 | — | — | 6.3 | 3.1 |
| Cd83 | 81 | — | — | — | 3.8 | 4.7 | 9.3 | 12.2 | 12.7 | 4.1 | 9.2 | — | 35.6 |
| Cd84 | 71 | — | — | — | 2.9 | 3.8 | 11.6 | 10.5 | 12.7 | 6.9 | 8.9 | 20.8 | 12.3 |
| Cd86 | 82 | — | — | — | 2.9 | 3.4 | 8.4 | 5.8 | 6.8 | 3.6 | 4.2 | 2.7 | 5.9 |
| Cd97 | 272 | — | — | — | 2.1 | 2.8 | 2.8 | 3.6 | 3.1 | 2.8 | 2.9 | 14.2 | 8.9 |
| Ptprc | 187 | — | 2.5 | — | 20.1 | 21.3 | 28.0 | 24.3 | 16.7 | 23.8 | 18.9 | 88.2 | 72.9 |
| (CD45) | | | | | | | | | | | | | |
| Sdc1 | 247 | — | — | — | — | — | 3.3 | 3.6 | 3.8 | 3.1 | 2.8 | — | — |
| (CD138) | | | | | | | | | | | | | |
| Thy1 | 132 | — | — | — | 9.2 | 10.6 | 7.6 | 11.2 | 5.7 | 12.0 | 11.6 | 71.9 | 91.6 |
| (CD90) | | | | | | | | | | | | | |

The table contains the signal strength for controls and fold changes for the transplants. (−) indicates that a given gene was not upregulated; bolded signal values indicate that a transcript was classified as present. In case of multiple probe sets querying the same gene, data obtained from probe sets with suffixes _s_at and _x_at were not considered, and a probe set displaying the most robust signal was selected.

Eighteen CD transcripts were present in normal kidney, perhaps reflecting immature dendritic cells in the interstitium (Austyn et al., J. Immunol., 152:2401-2410 (1994)). Expression of CD transcripts was similar between CTL and d4MLR. In addition, d4MLR contained the B cell specific transcripts CD79a and CD79b. Macrophage transcript CD14 was not expressed in CTL or d4MLR, while macrophage transcript CD68 was expressed at a low level in both.

Expression of CATs in Rejecting Mouse Kidney Allografts

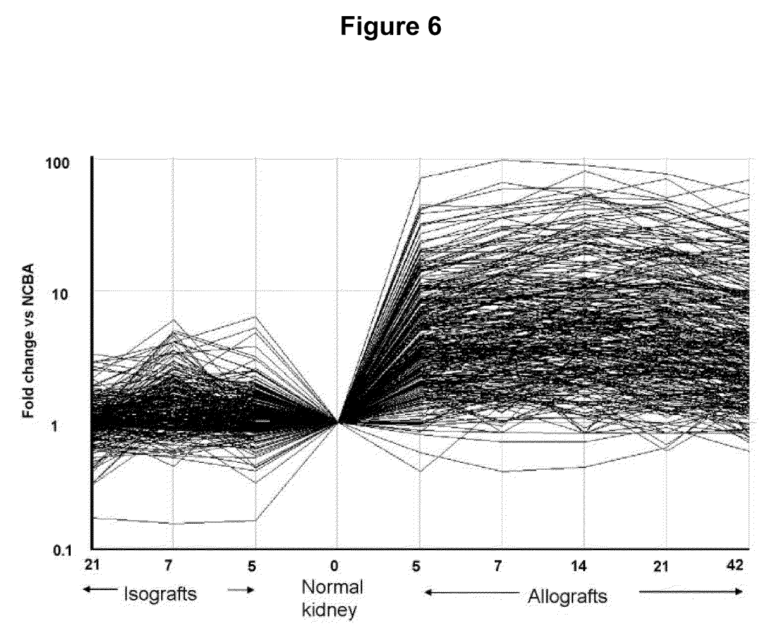

CATs were defined by high expression in both the CTL clone and in d4MLR but rated as “absent” in normal kidney. This algorithm identified 287 CATs. Expression of CATs was lower in d4MLR than in the CTL clone (mean 91±59 percent, median 87 percent). Compared to NCBA and isografts, the CATs were strongly expressed in rejecting WT allografts (FIG. 6). At day 5 post-transplant, the signal for CATs was increased 6.4 fold compared to NCBA and 14 percent (median) of that observed with the CTL clone (mean 20±28 percent). These results indicate that the CTL infiltrating the kidney are diluted about 5-6 fold compared to the CTL clone or the d4MLR. To confirm this interpretation, RNA from d4MLR was diluted with kidney RNA in a ratio 1:4. The resulting signal was similar to the signal in all rejecting kidneys (mean 20±7 percent, median 20 percent of the d4MLR and mean 18±11 percent, median 15 percent of expression in the CTL clone). Thus, at day 5, about one fifth to one sixth of the transcriptome of rejecting kidney is attributable to CATs. After day 5, mean expression of CATs was stable as a percent of the CTL signal (D7, 23.2±28 percent; D14, 27.3±45 percent; D21, 26.2±34 percent; and D42, 22.5±38 percent) and the median was also consistent (D5, 14 percent; D7, 16 percent; D14, 16 percent; D21, 16 percent; and D42, 12 percent).

To determine whether the pattern of CAT expression is consistent in vivo, the consistency of expression of individual CATs in various experimental conditions was analyzed. By non-parametric regression analysis, the expression of CATs correlated in all conditions, indicating robust maintenance of CAT expression in vivo and in vitro (Table 3). The d4MLR correlated well with the diluted MLR (r=0.91), despite the 80 percent decrease in signal, and slightly less well with the CTL clone (r=0.81; p<0.001). In rejecting transplants, the CAT signals exhibited a striking correlation among all days in wild-type hosts (r=0.90-0.96), indicating that most CATs displayed predictable and stable levels of expression in vivo in all rejecting kidneys. The correlations of d4MLR with the rejecting transplants at all days were considerably less (r=0.70-0.78; p<0.001), indicating significant differences between the relative transcript levels in vivo and in vitro. Expression in the CTL clone correlated least with that in the transplants (r=0.66-0.74; p=n.s.). Thus, the relative level of expression of individual CATs was similar in vitro between CTL and d4MLR, and was similar in vivo under all conditions in rejecting transplants, but was somewhat different in vivo compared to in vitro.

TABLE 3
Spearman rank correlations for CATs in lymphocytes from d4MLR and
a CTL clone (CTL), MLR diluted with kidney RNA 1:4 (MLRdil), and kidneys rejecting
in wild-type (WT) and B-cell deficient (IghKO) hosts at days 5-42 post transplant.

| | | | MLR | | | | | | IghKO | IghKO |
|---|---|---|---|---|---|---|---|---|---|---|
| | MLR | CTL | dil | WTD5 | WTD7 | WTD14 | WTD21 | WTD42 | D7 | D21 |
| MLR | 1 | .81 | .91 | .78 | .79 | .74 | .74 | .70 | .80 | .77 |
| CTL | .81 | 1 | .78 | .74 | .74 | .74 | .69 | .37 | .73 | .69 |
| MLRdil | .91 | .78 | 1 | .84 | .82 | .76 | .75 | .73 | .81 | .79 |
| WTD5 | .78 | .74 | .84 | 1 | .96 | .92 | .90 | .90 | .96 | .92 |
| WTD7 | .79 | .74 | .82 | .96 | 1 | .95 | .96 | .93 | .98 | .96 |
| WTD14 | .74 | .74 | .76 | .92 | .95 | 1 | .96 | .97 | .95 | .96 |
| WTD21 | .76 | .69 | .75 | .90 | .96 | .96 | 1 | .94 | .96 | .98 |
| WTD42 | .70 | .66 | .73 | .90 | .93 | .97 | .94 | 1 | .93 | .95 |
| IghKO | .80 | .73 | .81 | .96 | .98 | .95 | .96 | .93 | 1 | .97 |
| D7 | | | | | | | | | | |
| IghKO | .77 | .69 | .79 | .92 | .96 | .96 | .98 | .95 | .97 | 1 |
| D21 | | | | | | | | | | |

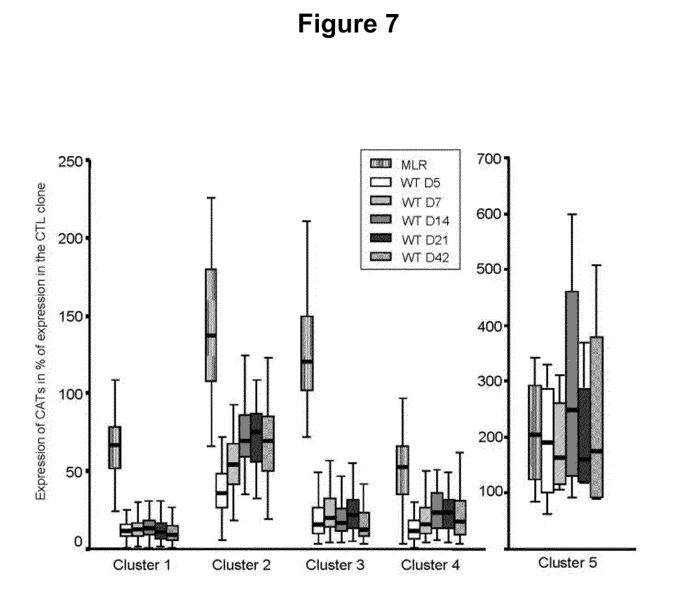

To further investigate expression patterns of individual CATs, a k-means cluster analysis of CATs was performed based on their expression level in wild-type allografts relative to the CTL clone. The 287 CATs were clustered into five clusters (FIG. 7). Cluster 1 has 140 transcripts (e.g., CD2, CD3g, GzmB, Tcrb, EOMES, and several genes related to the cell cycle) and was characterized by lower expression in d4MLR than CTL but relatively stable expression in all allografts (FIG. 7). The expression level for individual CATs are provided in Table 4. The mean expression was 6.1 fold increased versus NCBA at day 5, and remained unchanged thereafter. Cluster 2 has 23 transcripts (Table 4). The cluster 2 CATs were more highly expressed in d4MLR than CTL and relatively strongly increased in day 5 rejecting kidneys (6.7 fold; FIG. 7). A further 2.4 fold increase was observed from day 5 to day 14, and expression levels were stable thereafter. Cluster 3 has 74 transcripts, and the expression was also relatively high in d4MLR versus CTL, but lower in rejecting kidney, fluctuating somewhat among the different times (FIG. 7 and Table 4). Cluster 4 has 46 transcripts, and the CATs of this cluster were less expressed in d4MLR than CTL, exhibited a 2.2 fold increase in expression from day 5 to day 14, and exhibited a decreased expression thereafter by 1.4 fold. Cluster 5 has four transcripts, and the CATs of this cluster were as highly expressed in rejecting grafts as in the CTL clone and d4MLR (FIG. 7 and Table 4). Expression of CATs in cluster 2 and cluster 5 is higher than in clusters 1, 3, and 4, which contained the great majority of the CATs.

TABLE 4
CATs of clusters 1 through 5

| | | GenBank | | | | | | | | IghKO Allografts | | | |
|---|---|---|---|---|---|---|---|---|---|---|---|---|---|
| | | Accession | GenBank Accession Number | NCBA Signal | WT Allografts Fold Change | | | | | Fold Change | | Lymphocytes Fold Change | |
| Symbol | Gene Title | Number | for Human Ortholog | NCBA | D5 | D7 | D14 | D21 | D42 | D7 | D21 | CTL | MLRD4 |

CLUSTER 1

| | | | | | | | | | | | | | |
|---|---|---|---|---|---|---|---|---|---|---|---|---|---|
| Adam19 | a disintegrin and | D50410 | NM_023038 | 34 | 8.3 | 8.9 | 7.8 | 11.1 | 6.7 | 10.1 | 6.9 | 29.8 | 18.9 |
| | metalloproteinase domain | | NM_033274 | | | | | | | | | | |
| | 19 (meltrin beta) | | | | | | | | | | | | |
| Adam19 | a disintegrin and | NM_009616 | NM_023038 | 12 | 3.5 | 5.1 | 4.5 | 5.2 | 3.6 | 5.4 | 3.2 | 15.2 | 11.4 |
| | metalloproteinase domain | | NM_033274 | | | | | | | | | | |
| | 19 (meltrin beta) | | | | | | | | | | | | |
| Ask- | activator of S phase kinase | NM_013726 | NM_006716 | 72 | 3.6 | 3.5 | 3.8 | 3.2 | 2.8 | 3.4 | 2.1 | 22.3 | 18.5 |
| pending | | | | | | | | | | | | | |
| Aqp9 | aquaporin 9 | BC024105 | NM_020980 | 17 | 2.5 | 1.2 | 2.7 | 2.7 | 2.9 | 1.1 | 2.5 | 20.9 | 14.5 |
| Abcb9 | ATP-binding cassette, | AK020749 | NM_019624 | 8 | 1.6 | 1.3 | 1.1 | 1.0 | 0.8 | 1.7 | 1.8 | 19.6 | 16.0 |
| | sub-family B | BC017348 | NM_019625 | | | | | | | | | | |
| | (MDR/TAP), member 9 | | NM_203444 | | | | | | | | | | |
| | | | NM_203445 | | | | | | | | | | |
| | | | BC017348 | | | | | | | | | | |
| Brdg1- | BCR downstream | NM_019992 | NM_012108 | 130 | 1.5 | 1.6 | 2.2 | 1.5 | 1.6 | 1.6 | 1.4 | 10.5 | 8.6 |
| pending | signaling 1 | | BC014958 | | | | | | | | | | |
| Brca1 | breast cancer 1 | U31625 | AF005068 | 25 | 1.6 | 1.0 | 1.6 | 0.7 | 1.7 | 1.0 | 0.6 | 13.1 | 8.5 |
| | | | NM_007295 | | | | | | | | | | |
| Bub1 | budding uninhibited by | AF002823 | AF043294 | 9 | 5.7 | 5.6 | 6.9 | 4.8 | 4.8 | 7.0 | 4.1 | 77.7 | 55.9 |
| | benzimidazoles 1 homolog | | AK023540 | | | | | | | | | | |
| | (S. cerevisiae) | | | | | | | | | | | | |
| Bub1b | budding uninhibited by | NM_009773 | NM_001211 | 19 | 11.0 | 8.9 | 8.8 | 6.9 | 5.9 | 13.7 | 8.1 | 80.2 | 65.2 |
| | benzimidazoles 1 | | | | | | | | | | | | |
| | homolog, beta (S. cerevisiae) | | | | | | | | | | | | |
| Calmbp1 | calmodulin binding | BB648052 | AK001380 | 24 | 2.2 | 1.7 | 2.8 | 1.6 | 2.1 | 2.5 | 1.4 | 16.5 | 10.1 |
| | protein 1 | | | | | | | | | | | | |
| MGC38321 | CasL interacting molecule | BB209438 | NM_022765 | 10 | 5.3 | 6.5 | 6.7 | 8.4 | 5.7 | 6.4 | 8.1 | 62.6 | 44.4 |
| | MICAL | | | | | | | | | | | | |
| Ctsw | cathepsin W | NM_009985 | NM_001335 | 17 | 32.2 | 41.5 | 47.1 | 43.4 | 23.3 | 54.9 | 45.1 | 476.9 | 257.3 |
| Cd2 | CD2 antigen | NM_013486 | NM_001767 | 15 | 10.4 | 13.6 | 15.8 | 12.7 | 9.3 | 10.2 | 9.9 | 269.5 | 166.0 |
| Siva- | Cd27 binding protein | NM_013929 | AF033111 | 14 | 2.1 | 1.2 | 3.6 | 3.2 | 3.5 | 4.5 | 3.3 | 33.7 | 35.1 |
| pending | (Hindu God of | | NM_006427 | | | | | | | | | | |
| | destruction) | | AW024335 | | | | | | | | | | |
| Cd3g | CD3 antigen, gamma | M58149 | NM_000073 | 43 | 22.9 | 35.9 | 41.4 | 42.4 | 23.4 | 37.6 | 28.0 | 252.4 | 174.9 |
| | polypeptide | | | | | | | | | | | | |
| Cd53 | CD53 antigen | NM_007651 | NM_000560 | 134 | 11.4 | 17.3 | 22.6 | 18.2 | 19.6 | 18.0 | 13.0 | 73.9 | 71.7 |
| BC003314 | cDNA sequence | NM_030255 | NM_004900 | 86 | 8.6 | 9.8 | 8.6 | 9.5 | 7.9 | 12.5 | 8.5 | 44.4 | 35.1 |
| | BC003314 | | NM_145298 | | | | | | | | | | |
| | | | NM_021822 | | | | | | | | | | |
| Cdc6 | cell division cycle 6 | NM_011799 | NM_001254 | 11 | 5.6 | 3.0 | 5.2 | 3.5 | 3.2 | 4.7 | 2.4 | 44.0 | 19.0 |
| | homolog (S. cerevisiae) | | U77949 | | | | | | | | | | |
| Cenpa | centromere autoantigen A | AV132173 | NM_001809 | 22 | 10.5 | 10.4 | 10.0 | 8.3 | 6.0 | 10.2 | 6.0 | 180.3 | 166.7 |
| Chl12- | Chl12 homolog (yeast) | BM233289 | AK024476 | 5 | 0.9 | 0.8 | 0.8 | 0.8 | 0.8 | 0.9 | 0.9 | 12.4 | 10.5 |
| pending | | | | | | | | | | | | | |
| Hcapg- | chromosome condensation | AV277326 | NM_022346 | 5 | 4.4 | 3.0 | 4.7 | 2.7 | 3.8 | 3.5 | 1.6 | 73.1 | 22.0 |
| pending | protein G | | | | | | | | | | | | |
| Coro1a | coronin, actin binding | BB740218 | NM_007074 | 9 | 2.6 | 3.6 | 3.2 | 2.6 | 2.2 | 2.5 | 2.7 | 30.8 | 23.9 |
| | protein 1A | | | | | | | | | | | | |
| Ccna2 | cyclin A2 | NM_009828 | NM_001237 | 214 | 2.6 | 2.4 | 1.9 | 1.8 | 1.7 | 2.2 | 1.8 | 17.2 | 9.0 |
| Ccnb1 | cyclin B1 | AU015121 | NM_031966 | 15 | 12.4 | 11.0 | 12.1 | 7.2 | 4.1 | 8.5 | 6.0 | 109.3 | 51.0 |
| Ccnb2 | cyclin B2 | AK013312 | NM_004701 | 69 | 6.3 | 5.2 | 5.6 | 3.9 | 4.1 | 4.6 | 3.6 | 78.2 | 36.0 |
| | | | BF509102 | | | | | | | | | | |
| | | | AK023404 | | | | | | | | | | |
| | | | AU134430 | | | | | | | | | | |
| Ccnd2 | cyclin D2 | BM118679 | NM_001759 | 4 | 1.0 | 1.7 | 1.4 | 1.5 | 1.8 | 1.5 | 2.0 | 7.1 | 5.5 |
| Ccnd2 | cyclin D2 | BB840359 | NM_001759 | 9 | 2.0 | 1.4 | 3.0 | 1.2 | 2.6 | 1.1 | 1.2 | 8.1 | 7.1 |
| Ccne1 | cyclin E1 | NM_007633 | NM_001238 | 78 | 1.6 | 1.4 | 1.4 | 1.3 | 1.1 | 1.8 | 1.4 | 6.7 | 5.4 |
| | | | NM_057182 | | | | | | | | | | |
| Cst7 | cystatin F (leukocystatin) | NM_009977 | AF031824 | 15 | 15.3 | 23.5 | 28.7 | 25.9 | 22.4 | 31.9 | 23.0 | 223.5 | 199.5 |
| Diap3 | diaphanous homolog 3 | NM_019670 | NM_030932 | 6 | 1.7 | 2.0 | 2.7 | 1.1 | 2.3 | 2.1 | 1.0 | 20.8 | 9.8 |
| | (Drosophila) | | AL354829 | | | | | | | | | | |
| Dnmt1 | DNA methyltransferase | BB165431 | NM_001379 | 163 | 2.9 | 3.1 | 2.7 | 2.5 | 2.4 | 2.8 | 2.5 | 14.3 | 10.1 |
| | (cytosine-5) 1 | | | | | | | | | | | | |
| D2Ertd750e | DNA segment, Chr 2, | AK012148 | NM_033286 | 28 | 2.2 | 2.4 | 2.3 | 1.4 | 0.8 | 2.2 | 1.1 | 33.9 | 15.1 |
| | ERATO Doi 750, | | | | | | | | | | | | |
| | expressed | | | | | | | | | | | | |
| Emb | embigin | BG064842 | NM_198449 | 428 | 1.8 | 1.9 | 1.9 | 1.9 | 1.7 | 2.7 | 1.6 | 14.9 | 10.7 |
| Eomes | eomesodermin homolog | AB031037 | NM_005442 | 9 | 1.1 | 3.2 | 0.8 | 5.2 | 0.8 | 1.7 | 2.4 | 82.4 | 24.3 |
| | (Xenopus laevis) | | | | | | | | | | | | |
| | ESTs, Moderately similar | BM247465 | NM_024680 | 23 | 3.6 | 5.8 | 3.3 | 3.2 | 3.6 | 4.1 | 2.5 | 20.6 | 18.0 |
| | to hypothetical protein | | | | | | | | | | | | |
| | FLJ23311 [Homo sapiens] | | | | | | | | | | | | |
| | [H. sapiens] | | | | | | | | | | | | |
| Eef1b2 | eukaryotic translation | C77437 | NM_001008396 | 124 | 2.0 | 1.8 | 1.7 | 1.6 | 1.4 | 1.6 | 1.2 | 7.4 | 5.6 |
| | elongation factor 1 beta 2 | | NCBI | | | | | | | | | | |
| | | | NM_007086 | | | | | | | | | | |
| AA408511 | expressed sequence | AU018569 | AB040957 | 2 | 3.8 | 3.4 | 3.7 | 2.3 | 1.7 | 3.2 | 1.4 | 45.8 | 16.3 |
| | AA408511 | | NM_020890 | | | | | | | | | | |
| AA675320 | expressed sequence | BC025223 | NM_144595 | 100 | 1.4 | 1.5 | 1.9 | 1.4 | 1.2 | 1.6 | 1.4 | 8.5 | 6.9 |
| | AA675320 | | | | | | | | | | | | |
| AI173001 | expressed sequence | BC024727 | NM_014800 | 149 | 2.2 | 2.7 | 2.2 | 2.5 | 2.0 | 2.1 | 2.0 | 9.0 | 8.1 |
| | AI173001 | | NM_130442 | | | | | | | | | | |
| Fignl1 | fidgetin-like 1 | NM_021891 | NM_022116 | 10 | 9.6 | 9.8 | 8.4 | 7.4 | 3.8 | 10.6 | 5.4 | 77.4 | 53.0 |
| | | | AK023411 | | | | | | | | | | |
| Gtse1 | G two S phase expressed | NM_013882 | NM_016426 | 20 | 2.0 | 2.5 | 3.6 | 1.5 | 1.9 | 1.8 | 1.7 | 26.8 | 22.8 |
| | protein 1 | | BC006325 | | | | | | | | | | |
| | | | BF973178 | | | | | | | | | | |
| | | | AI218393 | | | | | | | | | | |
| | | | AI340239 | | | | | | | | | | |
| Glipr2 | GLI pathogenesis-related 2 | BM208214 | NM_022343 | 69 | 3.8 | 4.0 | 5.0 | 6.8 | 4.0 | 5.8 | 6.1 | 21.7 | 18.2 |
| Gzmb | granzyme B | NM_013542 | J03189 | 43 | 38.6 | 44.8 | 58.8 | 23.0 | 24.2 | 65.1 | 30.3 | 703.2 | 476.8 |
| Hemgn | hemogen | NM_053149 | AF130060 | 5 | 1.3 | 2.4 | 0.8 | 0.8 | 0.9 | 1.0 | 1.5 | 22.3 | 7.2 |
| | | | AF322875 | | | | | | | | | | |
| Hmmr | hyaluronan mediated | AF079222 | U29343 | 79 | 1.6 | 1.3 | 1.5 | 0.6 | 1.4 | 1.3 | 0.9 | 8.7 | 4.9 |
| | motility receptor | | NM_012485 | | | | | | | | | | |
| | (RHAMM) | | BC035392 | | | | | | | | | | |
| | | | BC002966 | | | | | | | | | | |
| | | | BM449961 | | | | | | | | | | |
| MGC37568 | hypothetical protein | BB327418 | BC006107 | 22 | 20.0 | 22.5 | 26.2 | 24.6 | 23.3 | 25.1 | 24.4 | 136.5 | 107.9 |
| | MGC37568 | | | | | | | | | | | | |
| Icos | inducible T-cell co- | AB023132 | AB023135 | 12 | 10.6 | 14.8 | 12.6 | 9.2 | 7.4 | 17.5 | 12.6 | 71.1 | 57.7 |
| | stimulator | | | | | | | | | | | | |
| Incenp | inner centromere protein | BQ175667 | NM_020238 | 60 | 4.2 | 4.4 | 3.3 | 4.0 | 3.0 | 4.6 | 3.0 | 18.7 | 14.6 |
| Incenp | inner centromere protein | BB418702 | NM_020238 | 85 | 5.1 | 5.3 | 5.0 | 5.5 | 4.0 | 5.1 | 3.5 | 59.0 | 19.6 |
| Il2ra | interleukin 2 receptor, | AF054581 | K03122 | 24 | 2.3 | 2.1 | 2.2 | 1.3 | 1.3 | 1.9 | 2.0 | 64.2 | 34.9 |
| | alpha chain | | NM_000417 | | | | | | | | | | |
| Il2rb | interleukin 2 receptor, beta | NM_008368 | NM_000878 | 24 | 23.8 | 30.9 | 32.9 | 39.5 | 26.2 | 32.4 | 30.2 | 168.8 | 119.7 |
| | chain | | | | | | | | | | | | |
| Il7r | interleukin 7 receptor | AI573431 | NM_002185 | 5 | 2.7 | 3.9 | 7.2 | 7.9 | 7.0 | 5.5 | 5.5 | 106.0 | 84.8 |
| | | | BE217880 | | | | | | | | | | |
| Kif10 | kinesin family member 10 | BG068387 | NM_001813 | 14 | 1.8 | 2.1 | 1.9 | 1.5 | 2.0 | 1.0 | 0.9 | 11.6 | 6.0 |
| Kif11 | kinesin family member 11 | BM234447 | NM_004523 | 46 | 3.8 | 3.0 | 2.9 | 2.2 | 2.2 | 2.6 | 1.7 | 39.1 | 13.4 |
| Kif11 | kinesin family member 11 | BB827235 | NM_004523 | 60 | 2.4 | 1.9 | 2.3 | 1.2 | 1.5 | 1.8 | 0.9 | 25.4 | 7.0 |
| Kif22-ps | kinesin family member 22, | BC003427 | NM_007317 | 7 | 3.6 | 2.4 | 3.2 | 2.5 | 1.9 | 4.2 | 1.2 | 37.2 | 15.6 |
| | pseudogene | | | | | | | | | | | | |
| Kif22-ps | kinesin family member 22, | BC003427 | NM_007317 | 14 | 4.0 | 3.6 | 5.4 | 2.7 | 3.9 | 4.1 | 2.5 | 65.2 | 24.7 |
| | pseudogene | | | | | | | | | | | | |
| Kif23 | kinesin family member 23 | BG082989 | NM_004856 | 3 | 1.4 | 1.0 | 1.3 | 0.8 | 1.6 | 0.9 | 0.9 | 16.5 | 10.4 |
| | | | NM_138555 | | | | | | | | | | |
| Kif23 | kinesin family member 23 | AW986176 | NM_004856 | 80 | 3.1 | 2.4 | 2.6 | 2.2 | 2.3 | 2.1 | 2.0 | 18.4 | 10.5 |
| | | | NM_138555 | | | | | | | | | | |
| Lmnb1 | lamin B1 | BG064054 | NM_005573 | 70 | 3.7 | 3.6 | 3.3 | 2.9 | 2.6 | 3.5 | 2.2 | 23.4 | 8.2 |
| Lek1 | leucine, glutamic acid, | BB049243 | NM_016343 | 152 | 1.1 | 1.5 | 1.4 | 1.3 | 0.7 | 1.5 | 1.2 | 11.5 | 7.4 |
| | lysine family 1 protein | | | | | | | | | | | | |
| Melk | maternal embryonic | NM_010790 | NM_014791 | 6 | 3.6 | 3.7 | 3.5 | 3.5 | 2.0 | 3.7 | 2.2 | 52.5 | 19.3 |
| | leucine zipper kinase | | | | | | | | | | | | |
| Ms4a4b | membrane-spanning 4- | BB199001 | n/a | 6 | 71.4 | 97.6 | 89.2 | 77.4 | 53.6 | 87.0 | 54.7 | 1238.4 | 519.1 |
| | domains, subfamily A, | | | | | | | | | | | | |
| | member 4B | | | | | | | | | | | | |
| Mcmd6 | mini chromosome | NM_008567 | NM_005915 | 285 | 3.1 | 3.2 | 3.4 | 2.9 | 2.3 | 3.4 | 2.5 | 18.7 | 13.2 |
| | maintenance deficient 6 | | | | | | | | | | | | |
| | (S. cerevisiae) | | | | | | | | | | | | |
| | Mus musculus adult male | BB014626 | n/a | 10 | 12.9 | 21.2 | 24.3 | 29.4 | 12.3 | 17.6 | 16.9 | 158.1 | 102.9 |
| | testis cDNA, RIKEN full- | | | | | | | | | | | | |
| | length enriched library, | | | | | | | | | | | | |
| | clone: 4930483L24 | | | | | | | | | | | | |
| | product: weakly similar to | | | | | | | | | | | | |
| | AT-HOOK PROTEIN | | | | | | | | | | | | |
| | AKNA [Homo sapiens], | | | | | | | | | | | | |
| | full insert sequence. | | | | | | | | | | | | |
| | Mus musculus, Similar to | BC026773 | AL832450 | 26 | 2.7 | 4.1 | 3.2 | 4.9 | 2.5 | 4.3 | 3.3 | 29.4 | 14.1 |
| | expressed sequence | | | | | | | | | | | | |
| | AI481279, clone | | | | | | | | | | | | |
| | MGC: 25733 | | | | | | | | | | | | |
| | IMAGE: 3982549, mRNA, | | | | | | | | | | | | |
| | complete cds | | | | | | | | | | | | |
| Myb | myeloblastosis oncogene | BC011513 | NM_005375 | 6 | 2.4 | 1.7 | 1.4 | 1.1 | 1.5 | 2.0 | 0.9 | 10.2 | 7.8 |
| Myb | myeloblastosis oncogene | NM_033597 | NM_005375 | 10 | 4.5 | 2.5 | 1.3 | 2.1 | 1.6 | 3.1 | 1.4 | 26.2 | 20.1 |
| Ncf4 | neutrophil cytosolic factor 4 | NM_008677 | NM_000631 | 122 | 5.6 | 6.1 | 7.8 | 6.2 | 6.9 | 7.0 | 6.0 | 17.3 | 14.7 |
| | | | NM_013416 | | | | | | | | | | |
| Np95 | nuclear protein 95 | NM_010931 | NM_013282 | 40 | 8.0 | 7.9 | 6.6 | 6.9 | 4.6 | 8.1 | 4.7 | 55.7 | 27.6 |
| Np95 | nuclear protein 95 | BB702754 | NM_013282 | 10 | 5.7 | 6.3 | 3.8 | 4.7 | 2.4 | 4.8 | 2.0 | 44.4 | 29.6 |
| Odf2 | outer dense fiber of sperm | AF000968 | AF053970 | 15 | 2.0 | 1.6 | 3.2 | 2.1 | 0.8 | 2.1 | 1.4 | 10.7 | 9.1 |
| | tails 2 | | AL138382 | | | | | | | | | | |
| Pvt1 | plasmacytoma variant | BE956863 | n/a | 31 | 0.6 | 0.4 | 0.5 | 0.6 | 2.0 | 0.8 | 0.8 | 8.9 | 9.1 |
| | translocation 1 | | | | | | | | | | | | |
| Plk | polo-like kinase homolog, | NM_011121 | NM_005030 | 102 | 2.9 | 2.8 | 2.5 | 1.9 | 2.0 | 2.5 | 1.8 | 19.7 | 14.7 |
| | (Drosophila) | | | | | | | | | | | | |
| Pole | polymerase (DNA | NM_011132 | NM_006231 | 7 | 2.8 | 4.5 | 4.2 | 2.6 | 1.2 | 4.1 | 3.6 | 53.4 | 36.5 |
| | directed), epsilon | | | | | | | | | | | | |
| Kcnn4 | potassium | NM_008433 | NM_002250 | 4 | 8.4 | 8.8 | 14.0 | 12.0 | 14.2 | 8.9 | 8.9 | 49.5 | 53.7 |
| | intermediate/small | | | | | | | | | | | | |
| | conductance calcium- | | | | | | | | | | | | |
| | activated channel, | | | | | | | | | | | | |
| | subfamily N, member 4 | | | | | | | | | | | | |
| Kcnn4 | potassium | BG865910 | NM_002250 | 18 | 19.8 | 19.7 | 32.4 | 26.3 | 31.5 | 23.8 | 25.4 | 123.4 | 97.3 |
| | intermediate/small | | | | | | | | | | | | |
| | conductance calcium- | | | | | | | | | | | | |
| | activated channel, | | | | | | | | | | | | |
| | subfamily N, member 4 | | | | | | | | | | | | |
| Pstpip1 | proline-serine-threonine | U87814 | AF038602 | 44 | 5.9 | 7.1 | 8.8 | 8.8 | 7.4 | 9.1 | 9.1 | 51.1 | 40.7 |
| | phosphatase-interacting | | | | | | | | | | | | |
| | protein 1 | | | | | | | | | | | | |
| Prss19 | protease, serine, 19 | NM_008940 | NM_007196 | 159 | 1.4 | 1.6 | 1.6 | 1.6 | 2.1 | 1.7 | 1.4 | 11.1 | 8.2 |
| | (neuropsin) | | NM_144505 | | | | | | | | | | |
| | | | NM_144506 | | | | | | | | | | |
| | | | NM_144507 | | | | | | | | | | |
| Prkcq | protein kinase C, theta | AB062122 | L01087 | 103 | 5.3 | 4.1 | 3.0 | 4.1 | 1.7 | 4.5 | 4.0 | 25.5 | 15.8 |
| | | | AK024876 | | | | | | | | | | |
| | | | AK024876 | | | | | | | | | | |
| | | | AL137145 | | | | | | | | | | |
| LOC233406 | protein regulator of | BC005475 | NM_003981 | 97 | 3.7 | 4.3 | 5.3 | 3.3 | 3.2 | 4.3 | 3.5 | 21.5 | 18.4 |
| | cytokinesis 1-like | | NM_199413 | | | | | | | | | | |
| | | | NM_199414 | | | | | | | | | | |
| Ptpn8 | protein tyrosine | NM_008979 | NM_015967 | 163 | 4.4 | 4.6 | 6.0 | 5.2 | 4.3 | 5.6 | 4.2 | 43.9 | 24.6 |
| | phosphatase, non-receptor | | NM_012411 | | | | | | | | | | |
| | type 8 | | AW665758 | | | | | | | | | | |
| Pycs | pyrroline-5-carboxylate | NM_019698 | NM_002860 | 87 | 3.3 | 2.7 | 3.9 | 3.5 | 4.0 | 3.3 | 3.1 | 8.0 | 8.7 |
| | synthetase (glutamate | | U76542 | | | | | | | | | | |
| | gamma-semialdehyde | | | | | | | | | | | | |
| | synthetase) | | | | | | | | | | | | |
| Pycs | pyrroline-5-carboxylate | BB833010 | NM_002860 | 13 | 4.4 | 5.6 | 8.1 | 4.4 | 7.2 | 3.3 | 4.9 | 15.0 | 14.5 |
| | synthetase (glutamate | | | | | | | | | | | | |
| | gamma-semialdehyde | | | | | | | | | | | | |
| | synthetase) | | | | | | | | | | | | |
| Racgap1 | Rac GTPase-activating | NM_012025 | AU153848 | 13 | 5.5 | 6.2 | 5.2 | 6.6 | 3.1 | 6.1 | 4.0 | 77.1 | 26.8 |
| | protein 1 | | | | | | | | | | | | |
| Racgap1 | Rac GTPase-activating | AF212320 | AU153848 | 164 | 2.1 | 2.2 | 2.1 | 1.7 | 1.5 | 2.3 | 1.8 | 21.2 | 7.9 |
| | protein 1 | | | | | | | | | | | | |
| Rad51ap1 | RAD51 associated protein 1 | NM_009013 | NM_006479 | 3 | 1.0 | 1.1 | 1.1 | 0.8 | 1.6 | 1.1 | 0.9 | 10.6 | 6.3 |
| Rad51 | RAD51 homolog (S. cerevisiae) | NM_011234 | D14134 | 8 | 14.6 | 12.8 | 12.3 | 9.7 | 8.8 | 12.4 | 7.5 | 96.8 | 51.6 |
| | | | NM_002875 | | | | | | | | | | |
| Rad541 | RAD54 like (S. cerevisiae) | AV310220 | NM_003579 | 45 | 3.6 | 4.0 | 4.0 | 3.3 | 3.2 | 4.2 | 2.9 | 17.8 | 13.9 |
| Rassf5 | Ras association | NM_018750 | AY062002 | 93 | 4.0 | 4.8 | 5.8 | 5.9 | 4.5 | 5.2 | 4.0 | 39.2 | 20.4 |
| | (RalGDS/AF-6) domain | | BC004270 | | | | | | | | | | |
| | family 5 | | | | | | | | | | | | |
| Rgs1 | regulator of G-protein | NM_015811 | S59049 | 28 | 6.9 | 8.0 | 11.9 | 11.2 | 9.6 | 8.4 | 9.1 | 82.1 | 73.1 |
| | signaling 1 | | NM_002922 | | | | | | | | | | |
| Rrm2 | ribonucleotide reductase | BF119714 | NM_001034 | 164 | 4.5 | 3.6 | 4.0 | 3.0 | 2.8 | 3.9 | 2.8 | 22.5 | 13.2 |
| | M2 | | | | | | | | | | | | |
| 1700054N08Rik | RIKEN cDNA | BC024705 | n/a | 15 | 2.5 | 1.2 | 3.6 | 2.8 | 0.8 | 4.6 | 3.0 | 14.0 | 10.4 |
| | 1700054N08 gene | | | | | | | | | | | | |
| 2310009O17Rik | RIKEN cDNA | BB799833 | NM_017447 | 28 | 4.0 | 3.7 | 7.1 | 3.3 | 5.3 | 3.8 | 3.1 | 55.6 | 30.4 |
| | 2310009O17 gene | | | | | | | | | | | | |
| 2310009O17Rik | RIKEN cDNA | BC019957 | NM_017447 | 214 | 1.6 | 1.8 | 2.3 | 2.3 | 1.8 | 1.9 | 2.0 | 8.3 | 6.2 |
| | 2310009O17 gene | | | | | | | | | | | | |
| 2310035M22Rik | RIKEN cDNA | NM_025863 | NM_173084 | 33 | 5.1 | 5.3 | 7.1 | 6.0 | 5.4 | 5.2 | 2.8 | 46.8 | 28.3 |
| | 2310035M22 gene | | | | | | | | | | | | |
| 2410003C07Rik | RIKEN cDNA | AK010351 | n/a | 16 | 10.9 | 10.1 | 11.2 | 6.7 | 5.3 | 9.6 | 6.1 | 91.6 | 40.6 |
| | 2410003C07 gene | | | | | | | | | | | | |
| 2410005L11Rik | RIKEN cDNA | BC022648 | NM_031423 | 17 | 3.2 | 2.6 | 2.0 | 2.5 | 2.1 | 2.4 | 2.1 | 25.3 | 16.7 |
| | 2410005L11 gene | | NM_145697 | | | | | | | | | | |
| 2600001J17Rik | RIKEN cDNA | BC006674 | n/a | 18 | 8.8 | 6.4 | 8.3 | 4.1 | 4.2 | 7.2 | 4.2 | 37.2 | 22.8 |
| | 2600001J17 gene | | | | | | | | | | | | |
| 2610020P18Rik | RIKEN cDNA | NM_023294 | NM_006101 | 47 | 1.9 | 1.8 | 2.3 | 1.7 | 1.8 | 2.0 | 1.3 | 19.2 | 6.9 |
| | 2610020P18 gene | | | | | | | | | | | | |
| 2610036L13Rik | RIKEN cDNA | NM_026410 | NM_080668 | 103 | 3.7 | 2.9 | 3.5 | 2.3 | 2.4 | 3.5 | 2.5 | 34.9 | 14.5 |
| | 2610036L13 gene | | | | | | | | | | | | |
| 2610201A12Rik | RIKEN cDNA | NM_133851 | NM_016359 | 103 | 3.4 | 3.3 | 3.3 | 2.5 | 2.3 | 3.4 | 2.3 | 51.0 | 26.5 |
| | 2610201A12 gene | | NM_018454 | | | | | | | | | | |
| | | | NM_002157 | | | | | | | | | | |
| 2610307O08Rik | RIKEN cDNA | AK012006 | NM_198282 | 183 | 6.8 | 6.4 | 6.0 | 6.4 | 7.3 | 6.2 | 5.9 | 6.8 | 6.8 |
| | 2610307O08 gene | | | | | | | | | | | | |
| 2610510J17Rik | RIKEN cDNA | BM230253 | NM_018455 | 52 | 3.5 | 3.2 | 3.5 | 2.6 | 2.5 | 3.3 | 2.7 | 14.0 | 9.5 |
| | 2610510J17 gene | | | | | | | | | | | | |
| 2810038K19Rik | RIKEN cDNA | NM_023684 | NM_017806 | 198 | 2.0 | 2.1 | 1.9 | 2.0 | 1.2 | 1.9 | 1.7 | 22.5 | 8.4 |
| | 2810038K19 gene | | | | | | | | | | | | |
| 3300001M08Rik | RIKEN cDNA | NM_028232 | NM_001012409 | 16 | 4.6 | 2.9 | 4.3 | 3.7 | 1.2 | 5.0 | 1.4 | 53.0 | 29.7 |
| | 3300001M08 gene | | NM_138484 | | | | | | | | | | |
| | | | NM_001012413 | | | | | | | | | | |
| 5730403J10Rik | RIKEN cDNA | BC004617 | n/a | 228 | 1.8 | 1.5 | 1.9 | 1.2 | 1.6 | 1.5 | 1.3 | 11.2 | 5.0 |
| | 5730403J10 gene | | | | | | | | | | | | |
| A430107P09Rik | RIKEN cDNA | X01134 | n/a | 448 | 6.2 | 8.5 | 8.3 | 8.0 | 4.3 | 9.9 | 6.1 | 45.4 | 30.0 |
| | A430107P09 gene | | | | | | | | | | | | |
| E430034C16Rik | RIKEN cDNA | NM_134163 | NM_018388 | 22 | 3.2 | 3.6 | 3.0 | 2.5 | 1.6 | 3.2 | 2.4 | 44.9 | 10.9 |
| | E430034C16 gene | | NM_133486 | | | | | | | | | | |
| Slfn1 | schlafen 1 | NM_011407 | n/a | 15 | 20.4 | 29.0 | 23.2 | 18.7 | 15.1 | 34.0 | 19.5 | 89.8 | 62.1 |
| 6-Sep | septin 6 | NM_019942 | D50918 | 27 | 3.0 | 4.1 | 3.5 | 2.8 | 2.7 | 3.8 | 2.0 | 30.9 | 23.2 |
| | | | D50918 | | | | | | | | | | |
| | | | AF403061 | | | | | | | | | | |
| | | | AK026589 | | | | | | | | | | |
| | | | T91323 | | | | | | | | | | |
| | | | AW150913 | | | | | | | | | | |
| | | | AI968130 | | | | | | | | | | |
| | | | AL568374 | | | | | | | | | | |
| Stk12 | serine/threonine kinase 12 | BC003261 | AB011446 | 18 | 5.4 | 5.2 | 5.3 | 3.6 | 4.1 | 4.0 | 2.9 | 48.3 | 18.8 |
| Stk18 | serine/threonine kinase 18 | BB706079 | NM_014264 | 35 | 1.8 | 1.6 | 2.3 | 1.5 | 1.2 | 1.6 | 1.0 | 11.4 | 6.2 |
| Stk4 | serine/threonine kinase 4 | NM_021420 | Z25430 | 12 | 4.7 | 3.3 | 6.4 | 3.3 | 4.4 | 2.7 | 3.2 | 20.7 | 13.6 |
| | | | NM_006282 | | | | | | | | | | |
| | | | BC039023 | | | | | | | | | | |
| | | | BC005231 | | | | | | | | | | |
| | | | BE222274 | | | | | | | | | | |
| | | | BF433725 | | | | | | | | | | |
| | | | AI763206 | | | | | | | | | | |
| Stk6 | serine/threonine kinase 6 | U80932 | NM_003600 | 115 | 2.2 | 2.3 | 2.2 | 1.6 | 1.8 | 2.0 | 1.5 | 14.0 | 10.0 |
| Sh2d1a | SH2 domain protein 1A | NM_011364 | AF072930 | 2 | 2.6 | 3.9 | 4.1 | 4.4 | 1.6 | 2.9 | 2.0 | 119.9 | 64.7 |
| | | | AF100540 | | | | | | | | | | |
| | | | AF100539 | | | | | | | | | | |
| | | | AF100542 | | | | | | | | | | |
| Sh2d2a | SH2 domain protein 2A | NM_021309 | NM_003975 | 22 | 14.4 | 20.0 | 18.9 | 21.1 | 10.1 | 21.9 | 14.0 | 109.9 | 68.7 |
| Sh3kbp1 | SH3-domain kinase | AK007283 | AF230904 | 39 | 5.6 | 8.5 | 10.7 | 7.9 | 9.4 | 8.9 | 5.8 | 68.6 | 56.9 |
| | binding protein 1 | | AF542051 | | | | | | | | | | |
| Sh3kbp1 | SH3-domain kinase | AK018032 | AF230904 | 70 | 3.1 | 3.9 | 5.8 | 3.6 | 4.4 | 4.2 | 2.5 | 26.2 | 16.7 |
| | binding protein 1 | | AF542051 | | | | | | | | | | |
| Slc28a2 | solute carrier family 28 | NM_172980 | NM_004212 | 8 | 10.4 | 15.8 | 21.3 | 16.0 | 16.0 | 17.9 | 11.7 | 64.3 | 56.1 |
| | (sodium-coupled | | | | | | | | | | | | |
| | nucleoside transporter), | | | | | | | | | | | | |
| | member 2 | | | | | | | | | | | | |
| Satb1 | special AT-rich sequence | AV172776 | NM_002971 | 28 | 3.2 | 2.8 | 3.3 | 3.0 | 1.9 | 4.2 | 2.9 | 111.1 | 85.1 |
| | binding protein 1 | | | | | | | | | | | | |
| Satb1 | special AT-rich sequence | BG092481 | NM_002971 | 26 | 0.8 | 0.7 | 0.7 | 0.9 | 0.6 | 0.8 | 0.8 | 30.5 | 17.7 |
| | binding protein 1 | | | | | | | | | | | | |
| Tcrb-V13 | T-cell receptor beta, | M16120 | n/a | 173 | 12.7 | 15.2 | 19.1 | 13.0 | 8.8 | 15.6 | 9.4 | 122.8 | 88.3 |
| | variable 13 | | | | | | | | | | | | |
| Tcrb-V13 | T-cell receptor beta, | U07661 | n/a | 94 | 20.7 | 24.0 | 22.8 | 19.3 | 13.5 | 21.3 | 15.6 | 142.0 | 97.1 |
| | variable 13 | | | | | | | | | | | | |
| Tcrb-V13 | T-cell receptor beta, | U46841 | n/a | 67 | 2.1 | 2.3 | 2.7 | 1.7 | 1.8 | 2.0 | 1.5 | 44.5 | 12.3 |
| | variable 13 | | | | | | | | | | | | |
| Tcrb-V13 | T-cell receptor beta, | X14388 | n/a | 9 | 14.4 | 15.5 | 16.5 | 13.8 | 8.5 | 15.9 | 8.5 | 301.5 | 71.4 |
| | variable 13 | | | | | | | | | | | | |
| Tcrb- | T-cell receptor beta, | BF658725 | n/a | 96 | 1.5 | 1.7 | 1.6 | 1.2 | 1.6 | 1.8 | 1.7 | 7.3 | 7.1 |
| V8.2 | variable 8.2 | | | | | | | | | | | | |
| Tk1 | thymidine kinase 1 | NM_009387 | NM_003258 | 168 | 2.0 | 2.0 | 1.9 | 1.8 | 1.3 | 2.3 | 2.0 | 10.9 | 9.7 |
| | | | BC007986 | | | | | | | | | | |
| Tyms | thymidylate synthase | BM068975 | NM_001071 | 17 | 0.9 | 1.0 | 1.1 | 1.0 | 1.5 | 0.7 | 0.7 | 8.3 | 4.7 |
| Trip13 | thyroid hormone receptor | AK010336 | NM_004237 | 18 | 3.2 | 2.8 | 2.5 | 2.0 | 2.4 | 2.7 | 2.1 | 18.1 | 15.1 |
| | interactor 13 | | | | | | | | | | | | |
| Traip | TRAF-interacting protein | NM_011634 | NM_005879 | 4 | 1.9 | 1.6 | 1.2 | 1.6 | 1.2 | 1.0 | 1.1 | 24.1 | 9.1 |
| Tacc3 | transforming, acidic | NM_011524 | NM_006342 | 6 | 3.8 | 3.6 | 4.0 | 2.8 | 2.9 | 3.9 | 2.4 | 43.6 | 24.2 |
| | coiled-coil containing | | AF289576 | | | | | | | | | | |
| | protein 3 | | | | | | | | | | | | |
| Tpp2 | tripeptidyl peptidase II | BB484264 | NM_003291 | 12 | 1.0 | 1.3 | 2.3 | 1.6 | 0.9 | 1.3 | 1.5 | 11.0 | 6.7 |
| Tnfrsf7 | tumor necrosis factor | L24495 | n/a | 9 | 6.5 | 12.2 | 6.4 | 7.6 | 5.3 | 12.8 | 7.9 | 51.8 | 42.1 |
| | receptor superfamily, | | | | | | | | | | | | |
| | member 7 | | | | | | | | | | | | |
| Ubl5 | ubiquitin-like 5 | AV210814 | NM_017703 | 10 | 1.0 | 0.8 | 0.8 | 1.3 | 1.3 | 1.1 | 1.3 | 14.0 | 5.4 |
| | | | AI479104 | | | | | | | | | | |
| Xlr4 | X-linked lymphocyte- | NM_021365 | N/a | 62 | 7.6 | 9.5 | 10.3 | 10.3 | 5.2 | 8.9 | 7.0 | 110.7 | 62.9 |
| | regulated 4 | | | | | | | | | | | | |
| Zap70 | zeta-chain (TCR) | NM_009539 | AB083211 | 33 | 13.9 | 20.2 | 16.3 | 18.0 | 10.6 | 19.7 | 13.4 | 90.0 | 60.0 |
| | associated protein kinase | | | | | | | | | | | | |
| Znfn1a1 | zinc finger protein, | NM_009578 | S80876 | 30 | 5.7 | 7.1 | 5.6 | 4.2 | 3.7 | 8.0 | 3.4 | 39.2 | 26.4 |
| | subfamily 1A, 1 (Ikaros) | | NM_006060 | | | | | | | | | | |
| | | NM_053213 | NM_031300 | 30 | 1.5 | 1.6 | 1.3 | 1.7 | 0.7 | 1.7 | 1.5 | 24.4 | 10.4 |
| | | AV126179 | NM_018131 | 8 | 2.0 | 0.8 | 1.9 | 0.8 | 0.8 | 1.1 | 1.2 | 19.7 | 12.1 |

CLUSTER 2

| | | | | | | | | | | | | | |
|---|---|---|---|---|---|---|---|---|---|---|---|---|---|
| Bcl2a1a | B-cell | L16462 | NM_004049 | 73 | 15.1 | 25.1 | 36.0 | 39.3 | 26.7 | 23.4 | 22.6 | 59.7 | 73.4 |
| | leukemia/lymphoma 2 | | | | | | | | | | | | |
| | related protein A1a | | | | | | | | | | | | |
| Ccl3 | chemokine (C-C motif) | NM_011337 | NM_002983 | 6 | 16.1 | 25.1 | 54.7 | 33.9 | 51.1 | 32.6 | 31.7 | 43.9 | 28.9 |
| | ligand 3 | | | | | | | | | | | | |
| Cd44 | CD44 antigen | X66083 | AF098641 | 5 | 16.3 | 16.0 | 53.4 | 31.8 | 41.0 | 23.6 | 16.5 | 61.3 | 48.2 |
| | | | M24915 | | | | | | | | | | |
| | | | NM_000610 | | | | | | | | | | |
| | | | BC004372 | | | | | | | | | | |
| Gadd45b | growth arrest and DNA- | AK010420 | AF087853 | 108 | 3.8 | 2.8 | 4.6 | 5.6 | 4.9 | 4.9 | 3.4 | 6.8 | 11.6 |
| | damage-inducible 45 beta | | AF078077 | | | | | | | | | | |
| | | | NM_015675 | | | | | | | | | | |
| | | | AV658684 | | | | | | | | | | |
| Ikbke | inhibitor of kappaB kinase | NM_019777 | NM_014002 | 10 | 5.1 | 12.7 | 10.9 | 20.2 | 15.3 | 16.9 | 18.5 | 18.6 | 92.1 |
| | epsilon | | AW340333 | | | | | | | | | | |
| Il10ra | interleukin 10 receptor, | NM_008348 | NM_001558 | 10 | 8.8 | 13.2 | 19.1 | 20.4 | 18.1 | 12.9 | 13.7 | 28.0 | 26.7 |
| | alpha | | | | | | | | | | | | |
| Il16 | interleukin 16 | BB167822 | NM_004513 | 42 | 0.4 | 1.4 | 2.3 | 2.2 | 1.3 | 2.1 | 1.2 | 6.7 | 11.8 |
| Il21r | interleukin 21 receptor | AB049137 | AF269133 | 16 | 7.0 | 9.5 | 8.5 | 10.8 | 8.1 | 8.7 | 8.8 | 21.8 | 70.4 |
| | | | NM_021798 | | | | | | | | | | |
| | | | AK093371 | | | | | | | | | | |
| Map3k8 | mitogen activated protein | NM_007746 | NM_005204 | 14 | 6.4 | 6.1 | 10.5 | 11.3 | 9.7 | 7.6 | 7.9 | 20.9 | 27.8 |
| | kinase 8 | | | | | | | | | | | | |
| Pglyrp | peptidoglycan recognition | NM_009402 | NM_005091 | 9 | 6.1 | 12.3 | 9.4 | 13.2 | 9.9 | 17.3 | 18.1 | 22.8 | 78.9 |
| | protein | | | | | | | | | | | | |
| Pim1 | proviral integration site 1 | AI323550 | n/a | 90 | 8.7 | 9.0 | 9.3 | 10.0 | 9.2 | 8.5 | 7.3 | 13.8 | 25.2 |
| Plek | pleckstrin | NM_019549 | NM_002664 | 41 | 17.8 | 29.9 | 28.7 | 30.7 | 28.0 | 33.6 | 25.5 | 40.5 | 40.6 |
| Runx1 | runt related transcription | NM_009821 | U19601 | 5 | 3.5 | 4.1 | 9.2 | 11.8 | 9.3 | 4.5 | 4.3 | 7.5 | 8.9 |
| | factor 1 | | D89788 | | | | | | | | | | |
| | | | L34598 | | | | | | | | | | |
| | | | NM_001754 | | | | | | | | | | |
| | | | S76346 | | | | | | | | | | |
| | | | D43968 | | | | | | | | | | |
| | | | D43967 | | | | | | | | | | |
| Tap1 | transporter 1, ATP- | BC024897 | n/a | 257 | 10.3 | 11.4 | 10.5 | 13.4 | 9.3 | 12.6 | 11.5 | 12.3 | 23.1 |
| | binding cassette, sub- | | | | | | | | | | | | |
| | family B (MDR/TAP) | | | | | | | | | | | | |
| Trim30 | tripartite motif protein 30 | BG068242 | n/a | 116 | 3.9 | 4.0 | 4.5 | 4.6 | 4.9 | 4.3 | 3.7 | 5.4 | 6.0 |
| 1300004C08Rik | RIKEN cDNA | AK004894 | L13852 | 61 | 4.6 | 4.7 | 9.1 | 6.8 | 7.3 | 6.0 | 5.4 | 7.8 | 10.7 |
| | 1300004C08 gene | | NM_003335 | | | | | | | | | | |
| 2610043M05Rik | RIKEN cDNA | BM247370 | NM_002719 | 20 | 0.9 | 2.5 | 6.2 | 7.3 | 7.3 | 3.8 | 4.5 | 14.3 | 11.6 |
| | 2610043M05 gene | | NCBI | | | | | | | | | | |
| | | | NM_178586 | | | | | | | | | | |
| | | | NCBI | | | | | | | | | | |
| | | | NM_178587 | | | | | | | | | | |
| | | | NCBI | | | | | | | | | | |
| | | | NM_178588 | | | | | | | | | | |
| 9030412M04Rik | RIKEN cDNA | AK018504 | NM_014737 | 38 | 3.5 | 5.1 | 5.3 | 6.6 | 6.7 | 4.9 | 5.0 | 7.6 | 13.3 |
| | 9030412M04 gene | | NM_170773 | | | | | | | | | | |
| | | | NCBI | | | | | | | | | | |
| | | | NM_170774 | | | | | | | | | | |
| E430025L02Rik | RIKEN cDNA | BC027411 | NM_198565 | 120 | 4.2 | 6.4 | 5.8 | 6.3 | 6.5 | 7.7 | 7.6 | 8.4 | 11.8 |
| | E430025L02 gene | | | | | | | | | | | | |
| MGC41320 | hypothetical protein | BC006817 | NM_025079 | 31 | 1.9 | 2.3 | 3.3 | 3.4 | 3.1 | 2.7 | 2.4 | 5.3 | 5.6 |
| | MGC41320 | | | | | | | | | | | | |
| | | BC003855 | n/a | 174 | 1.2 | 0.9 | 3.7 | 2.3 | 2.2 | 1.6 | 2.2 | 4.5 | 5.9 |
| | | BC003855 | n/a | 5 | 3.4 | 4.9 | 10.1 | 15.8 | 8.7 | 5.9 | 9.9 | 10.9 | 16.6 |
| | | BC003855 | n/a | 20 | 5.0 | 8.8 | 15.4 | 18.6 | 11.4 | 5.8 | 13.1 | 12.3 | 27.7 |

CLUSTER 3

| | | | | | | | | | | | | | |
|---|---|---|---|---|---|---|---|---|---|---|---|---|---|
| Abca7 | ATP-binding cassette, | NM_013850 | NM_019112 | 109 | 1.9 | 2.6 | 1.9 | 2.7 | 1.9 | 2.6 | 2.5 | 8.6 | 10.7 |
| | sub-family A (ABC1), | | | | | | | | | | | | |
| | member 7 | | | | | | | | | | | | |
| Apbblip- | amyloid beta (A4) | BC023110 | NM_019043 | 21 | 6.5 | 8.9 | 5.1 | 8.8 | 4.9 | 9.6 | 9.3 | 21.1 | 23.3 |
| pending | precursor protein-binding, | | BC035636 | | | | | | | | | | |
| | family B, member 1 | | | | | | | | | | | | |
| | interacting protein | | | | | | | | | | | | |
| Batf | basic leucine zipper | NM_016767 | NM_006399 | 31 | 10.0 | 11.6 | 9.6 | 11.3 | 7.2 | 8.3 | 8.7 | 20.4 | 23.5 |
| | transcription factor, ATF- | | | | | | | | | | | | |
| | like | | | | | | | | | | | | |
| Bcl11b | B-cell | NM_021399 | AB043584 | 17 | 6.0 | 8.6 | 5.4 | 9.1 | 3.2 | 7.0 | 8.3 | 101.8 | 100.7 |
| | lymphoma/leukaemia 11B | | NM_022898 | | | | | | | | | | |
| | | | AA918317 | | | | | | | | | | |
| | | | AU146285 | | | | | | | | | | |
| Brca1 | breast cancer 1 | U36475 | NM_007294 | 9 | 4.2 | 5.2 | 5.0 | 4.5 | 3.3 | 5.2 | 3.4 | 36.0 | 36.3 |
| | | | NCBI | | | | | | | | | | |
| | | | NM_007295 | | | | | | | | | | |
| | | | NCBI | | | | | | | | | | |
| | | | NM_007296 | | | | | | | | | | |
| | | | NCBI | | | | | | | | | | |
| | | | NM_007297 | | | | | | | | | | |
| | | | NCBI | | | | | | | | | | |
| | | | NM_007298 | | | | | | | | | | |
| | | | NCBI | | | | | | | | | | |
| | | | NM_007299 | | | | | | | | | | |
| | | | NCBI | | | | | | | | | | |
| | | | NM_007300 | | | | | | | | | | |
| | | | NCBI | | | | | | | | | | |
| | | | NM_007301 | | | | | | | | | | |
| | | | NCBI | | | | | | | | | | |
| | | | NM_007302 | | | | | | | | | | |
| | | | NCBI | | | | | | | | | | |
| | | | NM_007303 | | | | | | | | | | |
| | | | NCBI | | | | | | | | | | |
| | | | NM_007304 | | | | | | | | | | |
| | | | NCBI | | | | | | | | | | |
| | | | NM_007305 | | | | | | | | | | |
| | | | NCBI | | | | | | | | | | |
| | | | NM_007306 | | | | | | | | | | |
| Brca1 | breast cancer 1 | U31625 | AF005068 | 1 | 0.9 | 0.8 | 0.8 | 0.8 | 0.9 | 0.9 | 0.9 | 7.3 | 8.9 |
| | | | NM_007295 | | | | | | | | | | |
| Cd37 | CD37 antigen | BC019402 | NM_001774 | 21 | 9.9 | 15.8 | 14.4 | 21.3 | 11.0 | 18.5 | 11.9 | 106.1 | 127.4 |
| Cd3d | CD3 antigen, delta | NM_013487 | NM_000732 | 8 | 42.1 | 59.0 | 60.8 | 48.9 | 31.8 | 66.9 | 38.2 | 812.4 | 910.7 |
| | polypeptide | | | | | | | | | | | | |
| Cd3z | CD3 antigen, zeta | X84237 | J04132 | 4 | 4.1 | 4.7 | 5.7 | 6.0 | 2.2 | 5.8 | 3.8 | 56.8 | 70.3 |
| | polypeptide | | | | | | | | | | | | |
| Cep2 | centrosomal protein 2 | NM_008383 | NM_007186 | 11 | 1.1 | 2.5 | 0.8 | 3.8 | 0.8 | 2.9 | 3.3 | 11.9 | 17.8 |
| Elmo1 | engulfment and cell | BC006054 | NM_014800 | 15 | 5.3 | 6.1 | 5.3 | 5.0 | 3.0 | 5.3 | 4.1 | 20.7 | 25.4 |
| | motility 1, ced-12 | | NCBI | | | | | | | | | | |
| | homolog (C. elegans) | | NM_130442 | | | | | | | | | | |
| Fgf13 | fibroblast growth factor 13 | BC018238 | NM_004114 | | 2.7 | 3.1 | 1.6 | 2.9 | 1.1 | 4.2 | 2.1 | 15.4 | 12.9 |
| | | | NM_033642 | | | | | | | | | | |
| Foxm1 | forkhead box M1 | AK008037 | NM_033642 | | 1.4 | 1.8 | 1.1 | 1.3 | 1.2 | 1.4 | 1.4 | 6.4 | 6.2 |
| Gfi1 | growth factor independent 1 | NM_010278 | NM_005263 | 8 | 2.4 | 3.7 | 3.6 | 4.8 | 2.3 | 4.2 | 2.7 | 30.2 | 45.0 |
| Gzmc | granzyme C | NM_010371 | n/a | 6 | 1.0 | 1.9 | 3.0 | 2.0 | 2.9 | 1.3 | 2.3 | 24.1 | 35.1 |
| Ian4 | immune associated | NM_031247 | NM_018384 | 276 | 2.6 | 3.6 | 2.3 | 3.1 | 2.0 | 3.6 | 3.4 | 14.0 | 19.0 |
| | nucleotide 4 | | AL080068 | | | | | | | | | | |
| | | | AL080068 | | | | | | | | | | |
| Il12rb2 | interleukin 12 receptor, | NM_008354 | NM_001559 | 56 | 1.3 | 1.6 | 1.8 | 1.2 | 1.0 | 1.7 | 1.1 | 8.1 | 14.0 |
| | beta 2 | | | | | | | | | | | | |
| Il2ra | interleukin 2 receptor, | M30856 | NM_000417 | 76 | 1.8 | 1.6 | 1.1 | 1.2 | 1.1 | 1.7 | 1.0 | 11.2 | 22.0 |
| | alpha chain | | | | | | | | | | | | |
| Il2rg | interleukin 2 receptor, | L20048 | NM_000206 | 186 | 9.9 | 13.3 | 14.0 | 12.6 | 9.7 | 16.0 | 12.6 | 36.9 | 37.4 |
| | gamma chain | | | | | | | | | | | | |
| Irf4 | interferon regulatory | NM_013674 | NM_002460 | 15 | 7.4 | 7.1 | 6.7 | 11.0 | 6.6 | 9.2 | 5.6 | 50.2 | 102.7 |
| | factor 4 | | | | | | | | | | | | |
| Itgal | integrin alpha L | AF065902 | BC008777 | 67 | 3.7 | 5.6 | 4.7 | 5.6 | 2.8 | 6.4 | 4.4 | 28.2 | 24.9 |
| Itgb7 | integrin beta 7 | NM_013566 | NM_000889 | 30 | 14.0 | 21.1 | 16.0 | 22.6 | 11.4 | 23.1 | 20.3 | 48.5 | 96.2 |
| | | | AI807169 | | | | | | | | | | |
| Itk | IL2-inducible T-cell | NM_010583 | D13720 | 8 | 10.7 | 17.1 | 15.5 | 20.3 | 8.2 | 15.4 | 13.7 | 152.5 | 152.4 |
| | kinase | | | | | | | | | | | | |
| Itk | IL2-inducible T-cell | L10628 | D13720 | 17 | 2.2 | 3.5 | 3.4 | 4.6 | 1.4 | 4.5 | 1.7 | 33.2 | 33.6 |
| | kinase | | | | | | | | | | | | |
| Kcna3 | potassium voltage-gated | NM_008418 | NM_002232 | 48 | 1.3 | 1.6 | 1.2 | 1.4 | 1.4 | 1.6 | 1.2 | 4.7 | 6.4 |
| | channel, shaker-related | | | | | | | | | | | | |
| | subfamily, member 3 | | | | | | | | | | | | |
| Lat | linker for activation of T | AF036907 | AF036905 | 18 | 32.1 | 35.5 | 25.0 | 31.5 | 17.9 | 43.8 | 34.9 | 205.2 | 179.7 |
| | cells | | AF036906 | | | | | | | | | | |
| Lef1 | lymphoid enhancer | NM_010703 | AF294627 | 19 | 2.3 | 2.3 | 0.9 | 1.8 | 0.8 | 1.5 | 1.7 | 22.6 | 44.6 |
| | binding factor 1 | | AF288571 | | | | | | | | | | |
| | | | AW117601 | | | | | | | | | | |
| | | | AI762816 | | | | | | | | | | |
| Ltb | lymphotoxin B | NM_008518 | NM_002341 | 8 | 41.1 | 66.4 | 52.2 | 70.2 | 30.3 | 65.5 | 59.8 | 354.8 | 366.2 |
| Ly108 | lymphocyte antigen 108 | AF248636 | NM_052931 | 61 | 5.4 | 5.4 | 6.0 | 4.7 | 3.3 | 6.4 | 3.9 | 7.7 | 8.9 |
| Map4k1 | mitogen activated protein | BB546619 | NM_007181 | 7 | 11.5 | 13.1 | 11.0 | 15.0 | 7.5 | 12.2 | 10.4 | 56.3 | 81.1 |
| | kinase 1 | | | | | | | | | | | | |
| MGC37568 | hypothetical protein | AU043488 | BC006107 | 7 | 7.2 | 11.2 | 11.4 | 18.7 | 4.8 | 13.0 | 11.1 | 81.5 | 64.9 |
| | MGC37568 | | | | | | | | | | | | |
| MGC37914 | hypothetical protein | BC021614 | n/a | 89 | 2.6 | 3.4 | 2.4 | 3.0 | 1.5 | 3.4 | 2.4 | 21.9 | 20.7 |
| | MGC37914 | | | | | | | | | | | | |
| Ms4a4c | membrane-spanning 4- | NM_029499 | AF237912 | 136 | 8.2 | 8.3 | 7.0 | 4.0 | 3.4 | 8.5 | 4.4 | 16.7 | 24.7 |
| | domains, subfamily A, | | AF354928 | | | | | | | | | | |
| | member 4C | | NM_024021 | | | | | | | | | | |
| Myb | myeloblastosis oncogene | NM_033597 | NM_005375 | 5 | 9.2 | 7.9 | 4.1 | 5.8 | 2.2 | 6.0 | 3.2 | 59.7 | 50.0 |
| Nfatc1 | nuclear factor of activated | AK004810 | NM_006162 | 150 | 2.9 | 4.3 | 3.8 | 3.8 | 2.7 | 4.5 | 3.3 | 7.9 | 13.5 |
| | T-cells, cytoplasmic 1 | | NM_172387 | | | | | | | | | | |
| | | | NM_172388 | | | | | | | | | | |
| | | | NM_172389 | | | | | | | | | | |
| | | | NM_172390 | | | | | | | | | | |
| Pglyrpl- | peptidoglycan recognition | NM_021319 | BE672390 | 3 | 1.4 | 2.9 | 5.6 | 5.6 | 1.3 | 4.1 | 3.2 | 47.0 | 34.1 |
| pending | protein-like | | | | | | | | | | | | |
| Pik3cd | phosphatidylinositol 3- | NM_008840 | U57843 | 10 | 6.8 | 10.5 | 15.1 | 12.5 | 9.0 | 12.8 | 7.5 | 111.8 | 154.1 |
| | kinase catalytic delta | | U86453 | | | | | | | | | | |
| | polypeptide | | | | | | | | | | | | |
| Pik3cd | phosphatidylinositol 3- | BB700084 | n/a | 100 | 3.3 | 5.2 | 5.9 | 7.2 | 4.6 | 5.2 | 4.4 | 35.0 | 38.8 |
| | kinase catalytic delta | | | | | | | | | | | | |
| | polypeptide | | | | | | | | | | | | |
| Plxnc1 | plexin C1 | BB765457 | NM_005761 | 64 | 2.3 | 3.3 | 2.7 | 4.7 | 1.8 | 3.3 | 3.2 | 10.3 | 15.6 |
| Pom121 | nuclear pore membrane | C80273 | AK022555 | 66 | 2.2 | 2.5 | 2.3 | 3.4 | 2.1 | 2.5 | 2.2 | 6.4 | 6.9 |
| | protein 121 | | | | | | | | | | | | |
| Prkcb | protein kinase C, beta | BF660388 | NM_002738 | 6 | 5.3 | 7.3 | 6.5 | 11.3 | 4.1 | 7.2 | 5.6 | 13.2 | 18.9 |
| | | | NM_212535 | | | | | | | | | | |
| Rad51ap1 | RAD51 associated protein 1 | BC003738 | NM_006479 | 71 | 2.1 | 2.0 | 1.3 | 1.5 | 1.3 | 2.2 | 1.7 | 10.2 | 11.3 |
| Rgs10 | regulator of G-protein | NM_026418 | NM_002925 | 208 | 2.5 | 3.3 | 4.0 | 4.1 | 3.1 | 3.7 | 4.0 | 9.0 | 11.1 |
| | signaling 10 | | AI744627 | | | | | | | | | | |
| Rgs19 | regulator of G-protein | BC003838 | NM_005873 | 104 | 4.1 | 4.9 | 5.0 | 5.3 | 4.5 | 5.2 | 4.2 | 15.9 | 16.1 |
| | signaling 19 | | | | | | | | | | | | |
| Rog- | repressor of GATA | AK015881 | NM_014383 | 11 | 10.9 | 9.5 | 6.8 | 3.8 | 5.3 | 11.0 | 2.1 | 49.3 | 143.0 |
| pending | | | | | | | | | | | | | |
| Selpl | selectin, platelet (p- | NM_009151 | U02297 | 115 | 7.6 | 13.3 | 11.0 | 16.4 | 8.4 | 13.9 | 12.7 | 69.5 | 70.8 |
| | selectin) ligand | | | | | | | | | | | | |
| Sema4d | sema domain, | NM_013660 | NM_006378 | 149 | 2.3 | 2.8 | 2.6 | 2.4 | 1.4 | 3.5 | 2.5 | 8.0 | 10.4 |
| | immunoglobulin domain | | | | | | | | | | | | |
| | (Ig), transmembrane | | | | | | | | | | | | |
| | domain (TM) and short | | | | | | | | | | | | |
| | cytoplasmic domain, | | | | | | | | | | | | |
| | (semaphorin) 4D | | | | | | | | | | | | |
| Sh3bp1 | SH3-domain binding | NM_009164 | NM_018957 | 48 | 4.3 | 7.4 | 4.1 | 7.6 | 2.1 | 7.2 | 7.4 | 10.8 | 14.3 |
| | protein 1 | | AK024971 | | | | | | | | | | |
| Slc1a7 | solute carrier family 1, | NM_009201 | AF105230 | 216 | 1.5 | 1.8 | 1.4 | 2.1 | 1.6 | 2.0 | 1.4 | 6.6 | 7.4 |
| | member 7 | | BC000986 | | | | | | | | | | |
| Slc2a3 | solute carrier family 2 | M75135 | NM_006931 | 5 | 0.9 | 0.8 | 0.8 | 1.0 | 1.2 | 1.3 | 1.2 | 19.4 | 48.5 |
| | (facilitated glucose | | AL110298 | | | | | | | | | | |
| | transporter), member 3 | | | | | | | | | | | | |
| Stat4 | signal transducer and | NM_011487 | NM_003151 | 8 | 7.4 | 10.5 | 12.0 | 10.5 | 7.6 | 8.2 | 8.7 | 143.6 | 144.0 |
| | activator of transcription 4 | | | | | | | | | | | | |
| Stk10 | serine/threonine kinase 10 | NM_009288 | AB015718 | 53 | 4.5 | 5.8 | 4.6 | 6.9 | 2.8 | 5.9 | 3.4 | 32.1 | 28.6 |
| | | | NM_005990 | | | | | | | | | | |
| | | | BE504180 | | | | | | | | | | |
| | | | BE501281 | | | | | | | | | | |
| | | | AF088069 | | | | | | | | | | |
| Tacc3 | transforming, acidic | BB787809 | NM_006342 | 77 | 2.8 | 2.4 | 1.9 | 2.2 | 2.1 | 2.8 | 1.6 | 11.7 | 19.3 |
| | coiled-coil containing | | AF289576 | | | | | | | | | | |
| | protein 3 | | | | | | | | | | | | |
| Tcrb-V13 | T-cell receptor beta, | M87849 | n/a | 15 | 6.5 | 6.4 | 4.5 | 5.7 | 3.9 | 7.1 | 4.2 | 19.2 | 27.3 |
| | variable 13 | | | | | | | | | | | | |
| Tcrb- | T-cell receptor beta, | BF318536 | n/a | 24 | 2.3 | 3.6 | 2.4 | 2.8 | 2.7 | 3.6 | 2.9 | 18.4 | 35.4 |
| V8.2 | variable 8.2 | | | | | | | | | | | | |
| Trim34 | tripartite motif protein 34 | NM_030684 | AB039904 | 94 | 2.8 | 3.7 | 4.4 | 3.7 | 3.9 | 3.6 | 2.5 | 7.7 | 8.4 |
| | | | NM_021616 | | | | | | | | | | |
| 9-Sep | septin 9 | NM_017380 | AF142408 | 469 | 1.6 | 2.4 | 1.9 | 3.3 | 2.1 | 2.6 | 2.0 | 6.3 | 5.4 |
| | | | AB023208 | | | | | | | | | | |
| | | | NM_006640 | | | | | | | | | | |
| 2310021G01Rik | RIKEN cDNA | AK011289 | AY029179 | 11 | 8.5 | 7.8 | 5.0 | 5.2 | 2.0 | 8.3 | 4.2 | 24.9 | 49.8 |
| | 2310021G01 gene | | | | | | | | | | | | |
| 2700084L22Rik | RIKEN cDNA | NM_026024 | AB032931 | 5 | 2.5 | 3.5 | 3.1 | 1.9 | 1.3 | 2.0 | 1.6 | 30.5 | 36.7 |
| | 2700084L22 gene | | | | | | | | | | | | |
| 2810047L02Rik | RIKEN cDNA | AV270035 | NM_016448 | 28 | 3.3 | 3.5 | 3.2 | 3.1 | 2.0 | 4.1 | 2.5 | 24.9 | 27.9 |
| | 2810047L02 gene | | | | | | | | | | | | |
| 2810425K19Rik | RIKEN cDNA | BC025911 | AF121856 | 6 | 2.1 | 4.6 | 1.5 | 3.5 | 3.2 | 1.3 | 0.9 | 9.4 | 11.0 |
| | 2810425K19 gene | | NM_021249 | | | | | | | | | | |
| 3322402E17Rik | RIKEN cDNA | AK014382 | AB006628 | 6 | 1.1 | 1.3 | 0.8 | 1.5 | 1.6 | 1.7 | 1.3 | 13.8 | 34.5 |
| | 3322402E17 gene | | | | | | | | | | | | |
| 3322402E17Rik | RIKEN cDNA | BF730694 | AB006628 | 14 | 6.0 | 8.6 | 6.5 | 8.6 | 2.8 | 9.5 | 9.2 | 38.3 | 80.9 |
| | 3322402E17 gene | | | | | | | | | | | | |
| 5031419I10Rik | RIKEN cDNA | BB474868 | NM_016573 | 39 | 2.9 | 3.7 | 2.3 | 3.6 | 2.7 | 3.9 | 3.8 | 8.7 | 13.1 |
| | 5031419I10 gene | | | | | | | | | | | | |
| 5830400A04Rik | RIKEN cDNA | BM243660 | NM_004310 | 14 | 5.7 | 8.7 | 10.5 | 11.5 | 9.6 | 9.1 | 7.4 | 43.0 | 102.2 |
| | 5830400A04 gene | | | | | | | | | | | | |
| 9130017C17Rik | RIKEN cDNA | AF395844 | AK055837 | 74 | 3.7 | 4.5 | 4.2 | 4.2 | 3.7 | 4.0 | 4.1 | 7.8 | 7.7 |
| | 9130017C17 gene | | AW104269 | | | | | | | | | | |
| | | | AI081246 | | | | | | | | | | |
| | | | AA521424 | | | | | | | | | | |
| | | | AL161979 | | | | | | | | | | |
| A430104N18Rik | RIKEN cDNA | AA254104 | n/a | 25 | 4.0 | 5.5 | 6.6 | 7.0 | 6.0 | 3.9 | 4.8 | 72.9 | 125.6 |
| | A430104N18 gene | | | | | | | | | | | | |
| AA409164 | expressed sequence | BC006054 | n/a | 12 | 1.3 | 1.5 | 1.1 | 1.6 | 1.8 | 2.1 | 1.8 | 5.5 | 5.9 |
| | AA409164 | | | | | | | | | | | | |
| | | AK004668 | NM_012452 | 51 | 3.0 | 4.8 | 5.6 | 6.5 | 4.8 | 5.0 | 3.7 | 29.6 | 31.3 |
| | Mus musculus BIC | AY096003 | n/a | 3 | 1.7 | 1.7 | 2.7 | 2.4 | 2.2 | 1.8 | 1.1 | 8.3 | 20.6 |
| | noncoding mRNA, | | | | | | | | | | | | |
| | complete sequence. | | | | | | | | | | | | |
| | | BG976607 | n/a | 75 | 2.9 | 2.3 | 2.4 | 2.2 | 1.1 | 2.4 | 1.3 | 9.4 | 12.6 |
| | Mus musculus adult | AW557946 | NM_016457 | 61 | 2.8 | 3.3 | 3.7 | 4.8 | 2.6 | 3.3 | 3.4 | 18.5 | 22.3 |
| | female vagina cDNA, | | | | | | | | | | | | |
| | RIKEN full-length | | | | | | | | | | | | |
| | enriched library, | | | | | | | | | | | | |
| | clone: 9930101D06 | | | | | | | | | | | | |
| | product: PROTEIN | | | | | | | | | | | | |
| | KINASE D2 homolog | | | | | | | | | | | | |
| | [Homo sapiens], full insert | | | | | | | | | | | | |
| | sequence. | | | | | | | | | | | | |
| | Mus musculus 9 days | AW552536 | n/a | 10 | 3.4 | 2.9 | 2.6 | 4.0 | 2.1 | 3.2 | 3.0 | 20.5 | 27.2 |
| | embryo whole body | | | | | | | | | | | | |
| | cDNA, RIKEN full-length | | | | | | | | | | | | |
| | enriched library, | | | | | | | | | | | | |
| | clone: D030060F23 | | | | | | | | | | | | |
| | product: Mus musculus | | | | | | | | | | | | |
| | U22 snoRNA host gene | | | | | | | | | | | | |
| | (UHG) gene, complete | | | | | | | | | | | | |
| | sequence, full insert | | | | | | | | | | | | |
| | sequence. | | | | | | | | | | | | |
| | Mus musculus adult male | BB014626 | n/a | 3 | 7.5 | 13.6 | 8.7 | 22.2 | 4.7 | 17.4 | 12.1 | 69.3 | 75.2 |
| | testis cDNA, RIKEN full- | | | | | | | | | | | | |
| | length enriched library, | | | | | | | | | | | | |
| | clone: 4930483L24 | | | | | | | | | | | | |
| | product: weakly similar to | | | | | | | | | | | | |
| | AT-HOOK PROTEIN | | | | | | | | | | | | |
| | AKNA [Homo sapiens], | | | | | | | | | | | | |
| | full insert sequence. | | | | | | | | | | | | |

CLUSTER 4

| | | | | | | | | | | | | | |
|---|---|---|---|---|---|---|---|---|---|---|---|---|---|
| Adcy7 | adenylate cyclase 7 | BB746807 | NM_001114 | 73 | 8.2 | 12.6 | 20.1 | 16.9 | 12.5 | 14.2 | 12.7 | 53.2 | 34.4 |
| AV278559 | expressed sequence | BC026563 | AA668763 | 83 | 7.0 | 8.7 | 9.1 | 9.2 | 7.9 | 9.7 | 6.7 | 69.9 | 24.8 |
| | AV278559 | | | | | | | | | | | | |
| C4st2- | chondroitin 4- | NM_021528 | NM_018641 | 9 | 5.0 | 8.9 | 10.3 | 13.9 | 11.0 | 10.3 | 9.7 | 32.9 | 21.5 |
| pending | sulfotransferase 2 | | BC002918 | | | | | | | | | | |
| | | | BC029471 | | | | | | | | | | |
| | | | BC029471 | | | | | | | | | | |
| C79673 | expressed sequence | BG066664 | NM_031471 | 34 | 6.5 | 14.1 | 20.3 | 19.0 | 17.6 | 18.0 | 15.7 | 56.8 | 54.5 |
| | C79673 | | NM_178443 | | | | | | | | | | |
| Cd80 | CD80 antigen | AA596883 | NM_005191 | 31 | 1.5 | 1.0 | 1.9 | 2.3 | 1.7 | 1.2 | 1.6 | 9.8 | 5.2 |
| Cd8a | CD8 antigen, alpha chain | AK017889 | NM_001768 | 14 | 18.4 | 36.3 | 45.6 | 33.4 | 23.2 | 41.3 | 26.9 | 100.2 | 84.3 |
| | | | NM_171827 | | | | | | | | | | |
| Cd8b | CD8 antigen, beta chain | U34882 | AW296309 | 22 | 26.9 | 39.6 | 50.3 | 40.0 | 24.8 | 47.1 | 29.1 | 251.6 | 111.8 |
| | | | NM_172100 | | | | | | | | | | |
| | | | NM_004931 | | | | | | | | | | |
| Crmp1 | collapsin response | AB006714 | NM_001313 | 14 | 1.9 | 2.6 | 7.3 | 3.7 | 4.5 | 3.5 | 3.9 | 69.8 | 8.7 |
| | mediator protein 1 | | | | | | | | | | | | |
| Cxcr6 | chemokine (C—X—C motif) | NM_030712 | NM_006564 | 13 | 5.3 | 17.8 | 34.2 | 27.9 | 16.2 | 11.8 | 14.6 | 434.8 | 15.9 |
| | receptor 6 | | | | | | | | | | | | |
| Dock2 | dedicator of cyto-kinesis 2 | NM_033374 | D86964 | 19 | 17.1 | 28.9 | 36.4 | 46.6 | 23.9 | 28.9 | 30.0 | 200.5 | 116.4 |
| | | | BC016996 | | | | | | | | | | |
| E430024D12 | hypothetical protein | AV173260 | AI342543 | 6 | 5.2 | 8.7 | 10.8 | 10.3 | 10.1 | 10.7 | 9.1 | 155.5 | 66.1 |
| | E430024D12 | | | | | | | | | | | | |
| Evi2 | ecotropic viral integration | BB201368 | NM_006495 | 19 | 14.2 | 24.5 | 27.0 | 24.5 | 19.9 | 20.9 | 16.9 | 107.4 | 55.5 |
| | site 2 | | | | | | | | | | | | |
| Flt3l | FMS-like tyrosine kinase | L23636 | U03858 | 43 | 1.8 | 2.2 | 3.3 | 2.8 | 2.5 | 2.3 | 2.2 | 14.3 | 8.1 |
| | 3 ligand | | NM_001459 | | | | | | | | | | |
| Glipr2 | GLI pathogenesis-related 2 | AK017557 | NM_022343 | 17 | 11.1 | 11.5 | 23.6 | 20.7 | 14.2 | 23.0 | 16.3 | 118.4 | 67.9 |
| Gng2 | guanine nucleotide | BC021599 | NM_053064 | 14 | 10.3 | 16.5 | 28.0 | 15.7 | 24.0 | 17.7 | 14.1 | 182.8 | 45.9 |
| | binding protein (G | | | | | | | | | | | | |
| | protein), gamma 2 subunit | | | | | | | | | | | | |
| Gpr34 | G protein-coupled | NM_011823 | NM_005300 | 5 | 1.0 | 1.3 | 2.1 | 2.5 | 2.3 | 0.9 | 2.0 | 16.5 | 6.9 |
| | receptor 34 | | | | | | | | | | | | |
| Hcls1 | hematopoietic cell specific | NM_008225 | NM_005335 | 8 | 31.0 | 44.1 | 51.3 | 48.2 | 33.3 | 44.8 | 36.1 | 175.3 | 75.3 |
| | Lyn substrate 1 | | | | | | | | | | | | |
| Hcst | hematopoietic cell signal | AF172930 | AF285447 | 228 | 1.3 | 1.5 | 2.0 | 2.0 | 1.6 | 1.8 | 1.7 | 8.9 | 4.7 |
| | transducer | | | | | | | | | | | | |
| Il18r1 | interleukin 18 receptor 1 | NM_008365 | NM_003855 | 60 | 8.8 | 10.9 | 5.8 | 10.8 | 8.7 | 10.1 | 7.8 | 75.9 | 19.1 |
| Klrc1 | killer cell lectin-like | AF106008 | NM_002260 | 5 | 8.7 | 16.6 | 33.2 | 20.3 | 13.3 | 20.3 | 10.1 | 331.4 | 11.3 |
| | receptor subfamily C, | | NM_002261 | | | | | | | | | | |
| | member 1 | | | | | | | | | | | | |
| Klrd1 | killer cell lectin-like | NM_010654 | U30610 | 27 | 14.4 | 19.4 | 28.3 | 22.5 | 27.9 | 22.9 | 18.8 | 489.9 | 94.6 |
| | receptor, subfamily D, | | AB009597 | | | | | | | | | | |
| | member 1 | | NM_007334 | | | | | | | | | | |
| Ly75 | lymphocyte antigen 75 | NM_013825 | NM_002349 | 30 | 1.6 | 2.5 | 2.5 | 3.0 | 2.4 | 1.9 | 1.5 | 6.2 | 4.1 |
| Ly9 | lymphocyte antigen 9 | NM_008534 | NM_002348 | 7 | 8.2 | 13.3 | 22.9 | 19.4 | 19.7 | 18.4 | 16.5 | 82.2 | 42.8 |
| Myolg | myosin IG | BB235320 | NM_033054 | 98 | 7.2 | 8.4 | 10.6 | 11.0 | 6.3 | 8.7 | 8.1 | 70.3 | 29.0 |
| Pik3cg | phosphoinositide-3-kinase, | BB205102 | AF327656 | 20 | 2.7 | 3.9 | 6.3 | 5.8 | 3.9 | 3.7 | 3.1 | 19.8 | 11.3 |
| | catalytic, gamma | | NM_002649 | | | | | | | | | | |
| | polypeptide | | | | | | | | | | | | |
| Plcl2 | phospholipase C-like 2 | BM207017 | NM_015184 | 144 | 2.4 | 2.8 | 4.1 | 3.7 | 4.8 | 3.6 | 3.3 | 8.1 | 6.9 |
| Plek | pleckstrin | AF303745 | NM_002664 | 22 | 17.0 | 17.1 | 25.6 | 15.2 | 20.4 | 20.4 | 15.1 | 31.1 | 25.7 |
| Rgs16 | regulator of G-protein | U94828 | U94829 | 3 | 6.5 | 13.3 | 12.2 | 16.3 | 14.5 | 16.5 | 13.9 | 17.1 | 8.7 |
| | signaling 16 | | | | | | | | | | | | |
| Ripk3 | receptor-interacting | NM_019955 | NM_006871 | 48 | 5.5 | 6.8 | 11.7 | 7.4 | 10.1 | 8.2 | 6.3 | 27.6 | 15.1 |
| | serine-threonine kinase 3 | | | | | | | | | | | | |
| Runx2 | runt related transcription | D14636 | L40992 | 52 | 3.1 | 4.6 | 4.0 | 7.1 | 4.0 | 3.9 | 3.2 | 46.1 | 16.8 |
| | factor 2 | | NM_004348 | | | | | | | | | | |
| | | | NM_004348 | | | | | | | | | | |
| | | | AL353944 | | | | | | | | | | |
| Sla | src-like adaptor | NM_009192 | NM_006748 | 102 | 4.0 | 5.1 | 5.8 | 4.8 | 5.2 | 5.6 | 3.8 | 13.6 | 9.6 |
| Sla2 | Src-like-adaptor 2 | AF287467 | AF290986 | 24 | 1.5 | 8.1 | 6.3 | 5.9 | 2.9 | 6.6 | 3.1 | 23.5 | 15.7 |
| Sp100 | nuclear antigen Sp100 | U83636 | AF056322 | 89 | 2.5 | 2.9 | 3.5 | 3.5 | 2.5 | 3.3 | 2.7 | 28.1 | 10.0 |
| | | | U36501 | | | | | | | | | | |
| | | | U36501 | | | | | | | | | | |
| | | | NM_003113 | | | | | | | | | | |
| | | | NM_003113 | | | | | | | | | | |
| Tcrb-V13 | T-cell receptor beta, | U63547 | n/a | 5 | 1.5 | 3.0 | 3.4 | 2.3 | 1.2 | 2.2 | 1.4 | 26.6 | 7.8 |
| | variable 13 | | | | | | | | | | | | |
| Tcrg-V2 | T-cell receptor gamma, | X03802 | n/a | 22 | 1.9 | 2.1 | 2.8 | 4.6 | 3.1 | 1.9 | 3.1 | 16.7 | 11.8 |
| | variable 2 | | | | | | | | | | | | |
| Tnfsf6 | tumor necrosis factor | NM_010177 | AF288573 | 73 | 1.8 | 2.4 | 4.2 | 4.2 | 2.8 | 2.8 | 3.0 | 57.7 | 20.3 |
| | (ligand) superfamily, | | D38122 | | | | | | | | | | |
| | member 6 | | | | | | | | | | | | |
| Tpm3 | tropomyosin 3, gamma | NM_022314 | AF362887 | 16 | 5.7 | 4.2 | 6.3 | 4.2 | 4.8 | 4.0 | 2.9 | 26.0 | 8.9 |
| | | | AF362887 | | | | | | | | | | |
| | | | AY004867 | | | | | | | | | | |
| | | | BC000771 | | | | | | | | | | |
| | | | X04201 | | | | | | | | | | |
| Trex1 | three prime repair | NM_011637 | AJ243797 | 100 | 6.7 | 6.0 | 9.9 | 8.4 | 7.5 | 9.3 | 6.9 | 12.0 | 8.3 |
| | exonuclease 1 | | NM_130384 | | | | | | | | | | |
| | | | NM_016381 | | | | | | | | | | |
| | | | BC002903 | | | | | | | | | | |
| Trim12 | tripartite motif protein 12 | BM244351 | n/a | 3 | 2.0 | 3.7 | 7.1 | 5.4 | 3.1 | 4.0 | 3.1 | 30.6 | 17.2 |
| Vav1 | vav 1 oncogene | NM_011691 | NM_005428 | 7 | 4.7 | 7.2 | 7.2 | 8.2 | 8.6 | 7.3 | 5.8 | 26.3 | 17.3 |
| 2410004L22Rik | RIKEN cDNA | NM_029621 | NM_033417 | 23 | 4.5 | 7.0 | 7.6 | 7.3 | 5.4 | 6.3 | 6.3 | 24.8 | 16.8 |
| | 2410004L22 gene | | | | | | | | | | | | |
| 2810433K01Rik | RIKEN cDNA | NM_025581 | BF038461 | 2 | 1.0 | 1.4 | 2.3 | 1.2 | 1.0 | 1.1 | 0.9 | 33.2 | 9.2 |
| | 2810433K01 gene | | | | | | | | | | | | |
| 4930422C14 | hypothetical protein | BM241008 | n/a | 33 | 15.1 | 17.1 | 20.7 | 11.4 | 15.6 | 20.0 | 10.1 | 202.2 | 45.1 |
| | 4930422C14 | | | | | | | | | | | | |
| 9830126M18 | hypothetical protein | BM224662 | NM_019018 | 124 | 3.3 | 4.5 | 4.8 | 4.6 | 4.5 | 4.6 | 3.8 | 12.8 | 6.8 |
| | 9830126M18 | | | | | | | | | | | | |
| | | NM_011558 | n/a | 25 | 7.4 | 10.9 | 30.7 | 18.6 | 21.2 | 9.7 | 14.1 | 138.9 | 43.4 |
| | Mus musculus adult | BB204677 | NM_016457 | 81 | 1.7 | 1.8 | 2.7 | 2.3 | 2.8 | 2.0 | 1.6 | 5.8 | 5.7 |
| | female vagina cDNA, | | | | | | | | | | | | |
| | RIKEN full-length | | | | | | | | | | | | |
| | enriched library, | | | | | | | | | | | | |
| | clone: 9930101D06 | | | | | | | | | | | | |
| | product: PROTEIN | | | | | | | | | | | | |
| | KINASE D2 homolog | | | | | | | | | | | | |
| | [Homo sapiens], full insert | | | | | | | | | | | | |
| | sequence. | | | | | | | | | | | | |

CLUSTER 5

| | | | | | | | | | | | | | |
|---|---|---|---|---|---|---|---|---|---|---|---|---|---|
| Pdcd1 | programmed cell death 1 | NM_008798 | NM_005018 | 13 | 15.6 | 25.7 | 38.3 | 27.5 | 21.9 | 26.9 | 17.6 | 22.9 | 18.5 |
| Socs1 | suppressor of cytokine | AB000710 | AB005043 | 46 | 10.5 | 9.3 | 7.5 | 9.3 | 7.1 | 13.3 | 8.6 | 7.9 | 26.9 |
| | signaling 1 | | U88326 | | | | | | | | | | |
| Stat1 | signal transducer and | NM_009283 | NM_007315 | 359 | 17.1 | 15.0 | 23.2 | 14.1 | 18.1 | 15.9 | 13.8 | 7.1 | 11.7 |
| | activator of transcription 1 | | NM_139266 | | | | | | | | | | |
| | | BC002065 | n/a | 95 | 45.0 | 42.6 | 81.1 | 50.9 | 68.8 | 56.8 | 48.5 | 13.7 | 33.4 |

Numbers indicate signal strength for NCBA and fold changes versus NCBA for allografts and lymphocyte cultures.
The abbreviations are as follows:
NCBA = normal CBA kidney;
WT allografts = CBA kidneys rejecting in wild-type B6 hosts;
IghKO allografts = CBA kidneys rejecting in B-cell deficient B6 hosts;
CTL = CTL clone;
MLRD4 = mixed lymphocyte culture day 4;
D5 = day 5 post transplant.

Expression of CATs in Allografts Rejecting in B-Cell Deficient Hosts

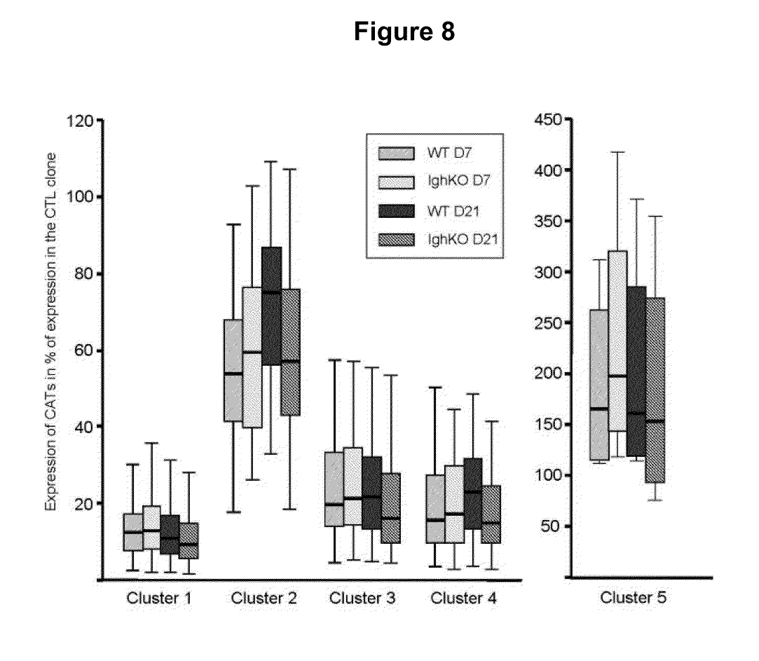

Whether the absence of B cells affects T-cell mediated rejection was analyzed by comparing CAT expression in kidneys rejecting in wild-type hosts to those rejecting in IghKO hosts at day 7 and day 21. The level of expression of CATs in grafts rejecting in IghKO hosts was highly correlated with that in wild-type hosts (D7: r=0.98; D21: r=0.98). The mean expression of the five clusters of CATs was also similar in IghKO versus wild-type hosts (FIG. 8), but was slightly higher in IghKO at day 7 (mean 23.2 percent in wild-type versus 25.3 percent in IghKO of the signal in the CTL clone) and lower in IghKO at day 21 (mean 26.2 percent in wild-type compared to the CTL clone versus 21.1 percent in IghKO).

In summary, the relationship between the pathologic Banff lesions of kidney rejection and the transcriptome, particularly in the CTL-associated transcripts, was studied. The interstitial infiltrate was established by day 5 and stable after day 7, whereas tubulitis and arteritis evolved slowly and progressively, being absent at day 5 and fully developed only after 14 days. The transcriptome changed markedly by day 5, with appearance of T cell and macrophage CD antigen transcripts. A set of CATs present in d4MLR and in a CTL clone but absent in normal kidney were identified. The CATs appeared in the transplants with a mean signal intensity about one fifth of that in the CTL clone, and was independent of B cells and alloantibody. In addition, CAT expression was essentially constant from day 5 through 42, despite massive changes in the histopathology. Thus, CTL transcripts appear early in rejecting kidneys, before the diagnostic Banff lesions, and persist for at least 6 weeks, providing a robust measurement of this aspect of rejection. This permits separation of the effectors of rejection, CTL, from the downstream consequences, parenchymal deterioration and pathologic lesions. In addition, CAT expression provides an approximation of the effector T cell burden and activity in rejecting kidneys. The interpretation of the CAT expression does not depend on the assumption that CATs are expressed exclusively in CTL, although it is likely that CTL account for most CAT expression.

The CD transcripts provide an overview of leukocyte population changes, and support the concept of a CTL and macrophage infiltrate with late B cell infiltration indicated by the histologic analysis. There is no real “gold standard” unbiased assessment of the composition of the infiltrate in rejecting transplants: both immunostaining of sections and cell isolation have potential for errors. Nevertheless, the arrays' estimates are fully compatible with estimates based on these methods. CD transcripts with high expression in CTL and d4MLR increased early during rejection and persisted throughout the time course, consistent with CTL infiltration and supporting the contention that CATs in the rejecting kidneys reflect transcripts in effector T cells. The macrophage markers CD14 and CD68 were present in rejecting kidneys, with low expression in CTL and d4MLR, consistent with macrophage infiltration. B cell markers CD79A and CD79B were present in d4MLR but not CTL, and appeared late in rejection, reflecting late B cell infiltration. There were few CD4⁺ cells in the infiltrate by immunostaining, and CD4 expression in the microarrays was low, in keeping with rejection being mainly driven by CD8⁺CTL.

The constancy of CAT expression over weeks establishes a new concept of T cell mediated rejection, namely that CTL generated from secondary lymphoid organs create and maintain a constant state in which the parenchyma progressively changes, yielding the pathologic lesions. The surprising stability of CAT levels over time suggests that the CTLs in the graft are occupying a finite “space,” similar to other emerging concepts of space in the secondary lymphoid organs (Stockinger et al., Immunology, 111(3):241-247 (2004)). The differences in the regression coefficients indicate that relative expression of individual CATs was consistent over time in vivo, although somewhat altered relative to the patterns of expression in vitro in the d4MLR and CTL clone. The moderate differences in relative expression of transcripts in the in vivo grafts versus the in vitro conditions may reflect different stimuli for CTL in these conditions (e.g., CD44). Other cells may also be recruited to express selected CAT in vivo: transcripts in cluster 5 exhibited high expression in vivo, perhaps reflecting IFN-γ effects (e.g., STAT1). The algorithm defining CATs, however, may exclude most IFN-γ inducible genes.

B cells do appear late in kidney rejection in this model but have no critical role, either as antigen presenting cells or alloantibody producing cells. Grafts in IghKO hosts exhibited very similar CAT expression to those in wild-type hosts by regression analysis, with slightly higher mean CAT expression at day 7 and lower at day 21. The small decline in CAT expression at day 21 in B cell deficient hosts suggest a role of B cells as second line antigen presenting cells sustaining CTL generation in secondary lymphoid organs.

The sustained expression of transcripts associated with cytotoxicity (e.g., perforin, granzymes A and B) in rejecting grafts raises the question of the role of cytotoxic mechanisms. Typical lesions develop in mice lacking perforin or granzyme A plus granzyme B (Halloran et al., Am. J. Transplant., 4(5):705-712 (2004)). Fas ligand (Tnfsf6) is expressed in CTL and rejecting grafts, but is not necessary for organ rejection across MHC disparities (Larsen et al., Transplant, 60(3):221-224 (1995)). Thus, the alterations in the parenchyma could reflect non-cytotoxic CTL and macrophage products, acting either by direct engagement or by indirect actions, e.g., extracellular matrix alterations triggering secondary changes in the epithelium. On the other hand, the lytic mechanisms such as perforin, granzymes, and Fas ligand could contribute to homeostasis, through fratricide of T cells (Huang et al., Science, 286(5441):952-954 (1999)) or interactions with antigen presenting cells (Ludewig et al., Eur. J. Immunol., 31(6):1772-1779 (2001)).

CAT expression can be used in estimating the burden of CTL in rejecting grafts, by analogy with viral load measurements in viral diseases. Moreover, although CD8⁻ CTL were used as the basis of the effector T cell signature, the definition of CATs probably includes most transcripts in CD4⁺ effector T cells. Less is known about effector CD4⁺ T cells in rejection, perhaps because CD8⁺ effectors develop more rapidly after short term stimulation (Seder and Ahmed, Nat. Immunol., 4(9):835-842 (2003)). CD4⁺ T cells may play a bigger role in human kidney allograft rejection than in mice, although in human rejection CD8⁺ T cells predominate (Hancock et al., Transplant, 35(5):458-463 (1983)). CD4⁺ effectors that home to inflammatory sites share many properties with CD8⁺ effectors, e.g., IFN-γ production, expression of P-selectin ligand and CXCR3, absence of CCR7 (Campbell et al., Nat. Immunol., 2(9):876-881 (2001)). Other transcript sets can be developed to reflect distinct events in a disease state, e.g., IFN-γ inducible transcripts or macrophage-associated transcripts.

### Example 2

### Kidney Rejection in Humans

### Human Database and Comparison with Mouse Transcripts

Data obtained from the mouse model were compared to the gene expression data obtained from human kidney biopsies from nine living donor controls, seven recipients with histologically confirmed acute rejection, five recipients with renal dysfunction without rejection on biopsy, and 10 protocol biopsies carried out more than one year post-transplant in patients with good transplant function and normal histology. Microarray data from these biopsies were obtained from a database available on the World Wide Web at scrips.edu/services/dna_array/. Flechner et al., Halloran laboratory Reference Manager #18134: Am. J. Transplant., 4(9):1475-1489 (2004)). Raw data were normalized as described herein for the mouse data, using the donor biopsies as controls. In GeneSpring, a homology database was created for the mouse and human data, and gene lists of interest were then used for supervised hierarchical clustering of the human biopsy samples.

CTL Gene Expression in Human Kidney Transplant Biopsies

The following was performed to determine whether or not the transcriptome pattern observed in mouse CTL and in rejecting mouse kidney reflects the rejection process in human transplant kidneys. A set of human kidney biopsies was analyzed based on the CTL signature identified in the mouse model. The database includes biopsies of normal kidneys (healthy donor biopsies), control biopsies of well functioning kidney transplants, rejecting transplants, and transplants with dysfunction but no rejection. The expression of CTL genes identified in mice in a published database of human renal transplants was examined. Of the 284 mouse CTL transcripts, 164 corresponding transcripts in the human database were identified. Supervised hierarchical cluster analysis based on the CTL transcripts separated the rejecting transplants from the other samples. In rejecting transplants, gene expression of CTL transcripts was increased compared to normal transplants with dysfunction but no rejection. Compared to donor biopsies, control biopsies of well functioning transplants had decreased expression of a subset of CTL transcripts, possibly due to immunosuppressive treatment. Another subset of transcripts exhibited increased expression in control biopsies, indicating some CTL activity in the transplant; however, expression levels were much lower than in rejecting kidneys. A class prediction model based on two classes (rejection-no rejection) identified 19 of the 21 samples correctly based on the expression of CTL transcripts in transplant biopsies (using the 100 best predictor genes (Fisher's Exact Test) and K-nearest neighbors (K=4)). The two samples that could not be classified were diagnosed as “borderline rejection” (AR5) and “tubular nephropathy” (NR5) based on histologic criteria.

In a first analysis of human kidney biopsies, the set of CTL genes identified in the mouse model exhibited striking upregulation in rejecting kidneys and permitted identification of samples from rejecting transplants without further refinement, indicating that the transcriptome patterns observed in rejecting mouse kidney reflect the rejection process in human transplant kidneys. Although this analysis includes only a limited number of human biopsies and may require verification and further refinement in a large patient population, this is a first indication that analysis of the CTL pattern in the transcriptome of kidney biopsies can be used as a diagnostic tool. Addition of other elements of the transcriptome to the CTL gene set may improve the diagnostic power, therefore allowing refinement of the gene set and reduction of the number of transcripts required for a diagnosis. The clinical application of this knowledge can involve either a microarray system using large numbers of genes or an RT-PCR system, depending on an evaluation of sensitivity, specificity, cost, and practicability. Based on the observation in the mouse model that transcriptome changes occur early before tubulitis develops, this approach can be more sensitive and quantitative than evaluation by histopathology and could be developed for use as an endpoint in clinical trials.

### Example 3

### CATs Identified Using a Second Algorithm

A second, more refined algorithm was used to identify CATs. This method involved RMA (robust multichip analysis). CATs were identified based on the following: a signal of less than 50 in normal kidneys in all three strains (CBA, B6, and Balbc); five times higher in CTL, MLR, and CD8 compared to normal kidneys; significantly (p (fdr)<0.01) higher in MLR versus normal kidney; two times increased in wild type allografts (CBA into B6) at day 5 compared to normal kidney; and significant in comparison to normal kidney (p(fdr)<0.01). This algorithm produced a list of 332 CATs, 91 of which were included in the original list of 287 CATs. The new list was checked for polymorphisms that would have been excluded if there had been any polymorphisms (5× difference between the strains or genes that are known to be highly polymorphic e.g., TCR, NKR, Ig, MHC). The list of 332 CATs is provided in Table 5.

TABLE 5
CATs identified using an RMA-based algorithm.

| | | | | | | Locus |
|---|---|---|---|---|---|---|
| Systematic | Symbol | Title | Genbank | Swissprot | Unigene | link |
| 1424965_at | Lpxn | leupaxin | BC026563 | 0 | Mm.313136 | 107321 |
| 1416016_at | Tap1 | transporter 1, ATP- | AW048052 | P21958, | Mm.207996 | 21354 |
| | | binding cassette, sub- | | Q62427, | | |
| | | family B | | Q62428, | | |
| | | (MDR/TAP) | | Q62429, | | |
| | | | | Q64333 | | |
| 1425226_x_at | Tcrb-V13 | T-cell receptor beta, | M16120 | 0 | Mm.333026 | 269846 |
| | | variable 13 | | | | |
| 1433935_at | AU020206 | expressed sequence | BI151331 | 0 | Mm.200422 | 101757 |
| | | AU020206 | | | | |
| 1419194_s_at | Gmfg | glia maturation | NM_022024 | 0 | Mm.194536 | 63986 |
| | | factor, gamma | | | | |
| 1451174_at | Lrrc33 | leucine rich repeat | BC027411 | 0 | Mm.33498 | 224109 |
| | | containing 33 | | | | |
| 1454169_a_at | Epstil | epithelial stromal | AK017174 | 0 | Mm.68134 | 108670 |
| | | interaction 1 (breast) | | | | |
| 1449127_at | Selpl | selectin, platelet (p- | NM_009151 | Q62170 | Mm.332590 | 20345 |
| | | selectin) ligand | | | | |
| 1436199_at | Trim14 | Tripartite motif- | AU042532 | 0 | Mm.240252 | 74735 |
| | | containing 14 | | | | |
| 1436423_at | E430004N04Rik | RIKEN cDNA | BE628523 | 0 | Mm.123021 | 210757 |
| | | E430004N04 gene | | | | |
| 1439595_at | Tcra | T-cell receptor alpha | BM243643 | 0 | Mm.213248 | 21473 |
| | | chain | | | | |
| 1452352_at | Ctla2b | cytotoxic T | BG064656 | 0 | 0 | 13025 |
| | | lymphocyte- | | | | |
| | | associated protein 2 | | | | |
| | | beta | | | | |
| 1437886_at | Klhl6 | kelch-like 6 | BM247104 | 0 | Mm.86699 | 239743 |
| | | (Drosophila) | | | | |
| 1460245_at | Klrd1 | killer cell lectin-like | NM_010654 | O54707, | Mm.8186 | 16643 |
| | | receptor, subfamily | | O54708 | | |
| | | D, member 1 | | | | |
| 1449925_at | Cxcr3 | chemokine (C—X—C | NM_009910 | O88410 | Mm.12876 | 12766 |
| | | motif) receptor 3 | | | | |
| 1436212_at | AI661017 | expressed sequence | AV173260 | 0 | Mm.132299 | 213068 |
| | | AI661017 | | | | |
| 1444088_at | | Similar to T-cell | BE447255 | P04212 | Mm.347827 | 381764 |
| | | receptor beta chain | | | | |
| | | VNDNJC precursor | | | | |
| 1440811_x_at | Cd8a | CD8 antigen, alpha | BB030365 | P01731, | Mm.1858 | 12525 |
| | | chain | | Q60965 | | |
| 1456064_at | AI504432 | expressed sequence | AI323624 | 0 | Mm.347584 | 229694 |
| | | AI504432 | | | | |
| 1448759_at | Il2rb | interleukin 2 | M28052 | P16297 | Mm.35287 | 16185 |
| | | receptor, beta chain | | | | |
| 1417597_at | Cd28 | CD28 antigen | NM_007642 | P31041 | Mm.255003 | 12487 |
| 1429270_a_at | 1700013H19Rik | RIKEN cDNA | AK005954 | 0 | Mm.229128 | 71846 |
| | | 1700013H19 gene | | | | |
| 1426025_s_at | Laptm5 | lysosomal-associated | U29539 | Q61168, | Mm.271868 | 16792 |
| | | protein | | Q60924 | | |
| | | transmembrane 5 | | | | |
| 1449220_at | Gimap3 | GTPase, IMAP | NM_031247 | 0 | Mm.333050 | 83408 |
| | | family member 3 | | | | |
| 1420876_a_at | 6-Sep | septin 6 | NM_019942 | 0 | Mm.260036 | 56526 |
| 1456494_a_at | Trim30 | tripartite motif | BG068242 | P15533 | Mm.295578 | 20128, |
| | | protein 30 | | | | 209387 |
| 1436570_at | | Transcribed locus | BG143461 | 0 | Mm.23897 | 0 |
| 1419178_at | Cd3g | CD3 antigen, gamma | M58149 | P11942 | Mm.335106 | 12502 |
| | | polypeptide | | | | |
| 1434280_at | | | BG976607 | 0 | 0 | 0 |
| 1448713_at | Stat4 | signal transducer and | NM_011487 | P42228 | Mm.1550 | 20849 |
| | | activator of | | | | |
| | | transcription 4 | | | | |
| 1417171_at | Itk | IL2-inducible T-cell | NM_010583 | Q03526 | Mm.339927 | 16428 |
| | | kinase | | | | |
| 1416118_at | | | NM_025863 | 0 | 0 | 0 |
| 1423760_at | Cd44 | CD44 antigen | M27130 | P15379 | Mm.330428 | 12505 |
| 1434929_at | BC035044 | cDNA sequence | BI076809 | 0 | Mm.373829 | 232406 |
| | | BC035044 | | | | |
| 1454764_s_at | | Transcribed locus | BF165681 | 0 | Mm.376972 | 0 |
| 1416956_at | Kcnab2 | potassium voltage- | U31908 | P62482 | Mm.302496 | 16498 |
| | | gated channel, | | | | |
| | | shaker-related | | | | |
| | | subfamily, beta | | | | |
| | | member 2 | | | | |
| 1417546_at | Il2rb | interleukin 2 | M28052 | P16297 | Mm.35287 | 16185 |
| | | receptor, beta chain | | | | |
| 1419569_a_at | Isg20 | interferon-stimulated | BC022751 | 0 | Mm.322843 | 57444 |
| | | protein | | | | |
| 1454850_at | Tbc1d10c | TBC1 domain family, | AV060417 | 0 | Mm.288312 | 108995 |
| | | member 10c | | | | |
| 1434380_at | | Diabetic | BM241271 | 0 | Mm.254851 | 0 |
| | | nephropathy-like | | | | |
| | | protein (Dnr12) | | | | |
| | | mRNA, partial | | | | |
| | | sequence | | | | |
| 1426396_at | Cd3z | CD3 antigen, zeta | AK017904 | P29020, | Mm.217308 | 12503 |
| | | polypeptide | | P24161 | | |
| 1443937_at | Il2rb | Interleukin 2 | BE634648 | P16297 | Mm.35287 | 16185 |
| | | receptor, beta chain | | | | |
| 1454893_at | 1110013L07Rik | RIKEN cDNA | BB765852 | 0 | Mm.274708 | 68521 |
| | | 1110013L07 gene | | | | |
| 1418842_at | Hcls1 | hematopoietic cell | NM_008225 | P49710 | Mm.4091 | 15163 |
| | | specific Lyn substrate 1 | | | | |
| 1425396_a_at | Lck | lymphocyte protein | BC011474 | P06240 | Mm.293753 | 16818 |
| | | tyrosine kinase | | | | |
| 1429197_s_at | Rabgap1l | RAB GTPase | BB431654 | 0 | Mm.25833 | 29809 |
| | | activating protein 1- | | | | |
| | | like | | | | |
| 1436097_x_at | Arhgap9 | Rho GTPase | BB327418 | 0 | Mm.227198 | 216445 |
| | | activating protein 9 | | | | |
| 1438439_at | Gpr171 | G protein-coupled | BB229616 | 0 | Mm.123648 | 229323 |
| | | receptor 171 | | | | |
| 1431592_a_at | Sh3kbp1 | SH3-domain kinase | AK007283 | 0 | Mm.286495 | 58194 |
| | | binding protein 1 | | | | |
| 1455899_x_at | Socs3 | suppressor of | BB241535 | O35718 | Mm.3468 | 12702 |
| | | cytokine signaling 3 | | | | |
| 1419193_a_at | Gmfg | glia maturation | NM_022024 | 0 | Mm.194536 | 63986 |
| | | factor, gamma | | | | |
| 1457725_at | 0 | Similar to membrane- | BB221406 | 0 | Mm.233909 | 381214 |
| | | spanning 4-domains, | | | | |
| | | subfamily A, member | | | | |
| | | 4C; membrane- | | | | |
| | | spanning 4-domains, | | | | |
| | | subfamily A, member 9 | | | | |
| 1434745_at | Ccnd2 | cyclin D2 | BQ175880 | P30280 | Mm.333406 | 12444 |
| 1423614_at | Lrrc8c | leucine rich repeat | BB329408 | 0 | Mm.319847 | 100604 |
| | | containing 8 family, | | | | |
| | | member C | | | | |
| 1427539_a_at | Zwint | ZW10 interactor | BC013559 | 0 | Mm.62876 | 52696 |
| 1454632_at | 6330442E10Rik | RIKEN cDNA | AV328515 | 0 | Mm.341747 | 268567 |
| | | 6330442E10 gene | | | | |
| 1424542_at | S100a4 | S100 calcium binding | D00208 | P07091 | Mm.3925 | 20198 |
| | | protein A4 | | | | |
| 1435331_at | AI447904 | expressed sequence | BM241008 | 0 | Mm.360525 | 236312 |
| | | AI447904 | | | | |
| 1448441_at | Cks1b | CDC28 protein | NM_016904 | P61025 | Mm.3049 | 54124 |
| | | kinase 1b | | | | |
| 1436171_at | Arhgap30 | Rho GTPase | BM244999 | 0 | Mm.251048 | 226652 |
| | | activating protein 30 | | | | |
| 1455576_at | 5830482F20Rik | RIKEN cDNA | AW493583 | 0 | Mm.74632 | 320435 |
| | | 5830482F20 gene | | | | |
| 1417104_at | Emp3 | epithelial membrane | BC001999 | O35912 | Mm.20829 | 13732 |
| | | protein 3 | | | | |
| 1424727_at | Ccr5 | chemokine (C-C | D83648 | P51682 | Mm.14302 | 12774 |
| | | motif) receptor 5 | | | | |
| 1419033_at | 2610018G03Rik | RIKEN cDNA | AW556821 | 0 | Mm.377135 | 70415 |
| | | 2610018G03 gene | | | | |
| 1416246_a_at | Coro1a | coronin, actin binding | BC002136 | O89053 | Mm.290482 | 12721 |
| | | protein 1A | | | | |
| 1439956_at | 0 | Adult male aorta and | BE692425 | 0 | Mm.123404 | 0 |
| | | vein cDNA, RIKEN | | | | |
| | | full-length enriched | | | | |
| | | library, | | | | |
| | | clone: A530049N04 | | | | |
| | | product: unknown | | | | |
| | | EST, full insert | | | | |
| | | sequence | | | | |
| 1433466_at | AI467606 | expressed sequence | BB234337 | 0 | Mm.284102 | 101602 |
| | | AI467606 | | | | |
| 1424560_at | Pstpip1 | proline-serine- | U87814 | P97814 | Mm.2534 | 19200 |
| | | threonine | | | | |
| | | phosphatase- | | | | |
| | | interacting protein 1 | | | | |
| 1425947_at | Ifng | interferon gamma | K00083 | P01580 | Mm.240327 | 15978 |
| 1460338_a_at | Crlf3 | cytokine receptor-like | BB161253 | 0 | Mm.272093 | 54394 |
| | | factor 3 | | | | |
| 1450698_at | Dusp2 | dual specificity | L11330 | Q05922 | Mm.4729 | 13537 |
| | | phosphatase 2 | | | | |
| 1438052_at | A130071D04Rik | RIKEN cDNA | BM239436 | 0 | 0 | 320791 |
| | | A130071D04 gene | | | | |
| 1425335_at | Cd8a | CD8 antigen, alpha | M12825 | P01731, | Mm.1858 | 12525 |
| | | chain | | Q60965 | | |
| 1455898_x_at | Slc2a3 | solute carrier family | BB414515 | P32037, | Mm.269857 | 20527 |
| | | 2 (facilitated glucose | | Q61607 | | |
| | | transporter), member 3 | | | | |
| 1419135_at | Ltb | lymphotoxin B | NM_008518 | P41155 | Mm.1715 | 16994 |
| 1416022_at | Fabp5 | fatty acid binding | BC002008 | Q05816 | Mm.741 | 16592 |
| | | protein 5, epidermal | | | | |
| 1434873_a_at | Centb1 | centaurin, beta 1 | BB115902 | 0 | Mm.288671 | 216859 |
| 1460419_a_at | Prkcb1 | protein kinase C, beta 1 | X59274 | P68404 | Mm.207496 | 18751 |
| 1441677_at | Smc4l1 | SMC4 structural | BM244144 | 0 | Mm.206841 | 70099 |
| | | maintenance of | | | | |
| | | chromosomes 4-like | | | | |
| | | 1 (yeast) | | | | |
| 1448500_a_at | Lime1 | Lck interacting | NM_023684 | 0 | Mm.272712 | 72699 |
| | | transmembrane | | | | |
| | | adaptor 1 | | | | |
| 1447788_s_at | AW212607 | expressed sequence | BB308532 | 0 | Mm.277243 | 241732 |
| | | AW212607 | | | | |
| 1424927_at | Glipr1 | GLI pathogenesis- | BC025083 | 0 | Mm.173790 | 73690 |
| | | related 1 (glioma) | | | | |
| 1455000_at | Gpr68 | G protein-coupled | BB538372 | 0 | Mm.32160 | 238377 |
| | | receptor 68 | | | | |
| 1439034_at | Spn | sialophorin | BB160586 | 0 | Mm.283714 | 20737 |
| 1425854_x_at | Tcrb-V13 | T-cell receptor beta, | U07661 | 0 | Mm.333026 | 269846 |
| | | variable 13 | | | | |
| 1418126_at | Ccl5 | chemokine (C-C | NM_013653 | P30882 | Mm.284248 | 20304 |
| | | motif) ligand 5 | | | | |
| 1437176_at | LOC434341 | similar to nucleotide- | AV277444 | 0 | 0 | 434341 |
| | | binding | | | | |
| | | oligomerization | | | | |
| | | domains 27 | | | | |
| 1424278_a_at | Birc5 | baculoviral IAP | BC004702 | O70201 | Mm.8552 | 11799 |
| | | repeat-containing 5 | | | | |
| 1424923_at | Serpina3g | serine (or cysteine) | BC002065 | Q62259 | Mm.264709 | 20715 |
| | | proteinase inhibitor, | | | | |
| | | clade A, member 3G | | | | |
| 1435529_at | 0 | Brain CRL-1443 | BM245961 | 0 | Mm.371956 | 0 |
| | | BC3H1 cDNA, | | | | |
| | | RIKEN full-length | | | | |
| | | enriched library, | | | | |
| | | clone: G430091H17 | | | | |
| | | product: weakly | | | | |
| | | similar to | | | | |
| | | GLUCOCORTICOID- | | | | |
| | | ATTENUATED | | | | |
| | | RESPONSE GENE | | | | |
| | | 16 PRODUCT | | | | |
| | | [Rattus norvegicus], | | | | |
| | | full insert sequence | | | | |
| 1416296_at | Il2rg | interleukin 2 | L20048 | P34902 | Mm.2923 | 16186 |
| | | receptor, gamma | | | | |
| | | chain | | | | |
| 1424181_at | 38966 | septin 6 | BC010489 | 0 | Mm.260036 | 56526 |
| 1451099_at | Mbc2 | membrane bound C2 | BC011482 | 0 | Mm.66056 | 23943 |
| | | domain containing | | | | |
| | | protein | | | | |
| 1426652_at | Mcm3 | minichromosome | BI658327 | P25206 | Mm.4502 | 17215 |
| | | maintenance deficient | | | | |
| | | 3 (S. cerevisiae) | | | | |
| 1416869_x_at | Lime1 | Lck interacting | NM_023684 | 0 | Mm.272712 | 72699 |
| | | transmembrane | | | | |
| | | adaptor 1 | | | | |
| 1452954_at | Ube2c | ubiquitin-conjugating | AV162459 | 0 | Mm.89830 | 68612 |
| | | enzyme E2C | | | | |
| 1440196_at | 0 | 3 days neonate | BB207611 | 0 | Mm.1891 | 0 |
| | | thymus cDNA, | | | | |
| | | RIKEN full-length | | | | |
| | | enriched library, | | | | |
| | | clone: A630020E03 | | | | |
| | | product: unknown | | | | |
| | | EST, full insert | | | | |
| | | sequence | | | | |
| 1452117_a_at | Fyb | FYN binding protein | BB157866 | O35601 | Mm.170905 | 23880 |
| 1450842_a_at | Cenpa | centromere | AV132173 | O35216 | Mm.290563 | 12615 |
| | | autoantigen A | | | | |
| 1427325_s_at | AI597013 | expressed sequence | BB014626 | 0 | Mm.258930 | 100182 |
| | | AI597013 | | | | |
| 1437432_a_at | Trim12 | tripartite motif | BM244351 | 0 | Mm.327033 | 76681 |
| | | protein 12 | | | | |
| 1418980_a_at | Cnp1 | cyclic nucleotide | M58045 | P16330 | Mm.15711 | 12799 |
| | | phosphodiesterase 1 | | | | |
| 1427007_at | 1200013B08Rik | RIKEN cDNA | AK004734 | 0 | Mm.276131 | 74131 |
| | | 1200013B08 gene | | | | |
| 1435945_a_at | Kcnn4 | potassium | BG865910 | O89109 | Mm.9911 | 16534 |
| | | intermediate/small | | | | |
| | | conductance calcium- | | | | |
| | | activated channel, | | | | |
| | | subfamily N, member 4 | | | | |
| 1451910_a_at | Cd6 | CD6 antigen | U12434 | Q61003 | Mm.290897 | 12511 |
| 1422808_s_at | Dock2 | dedicator of cytokinesis 2 | NM_033374 | 0 | Mm.217288 | 94176 |
| 1423895_a_at | Cugbp2 | CUG triplet repeat, | BB644164 | 0 | Mm.147091 | 14007 |
| | | RNA binding protein 2 | | | | |
| 1418770_at | Cd2 | CD2 antigen | NM_013486 | P08920 | Mm.22842 | 12481 |
| 1418465_at | Ncf4 | neutrophil cytosolic | NM_008677 | P97369 | Mm.2068 | 17972 |
| | | factor 4 | | | | |
| 1418641_at | Lcp2 | lymphocyte cytosolic | BC006948 | Q60787 | Mm.265350 | 16822 |
| | | protein 2 | | | | |
| 1448409_at | Lrmp | lymphoid-restricted | NM_008511 | Q60664 | Mm.843 | 16970 |
| | | membrane protein | | | | |
| 1436953_at | Waspip | Wiskott-Aldrich | C76969 | 0 | Mm.223504 | 215280 |
| | | syndrome protein | | | | |
| | | interacting protein | | | | |
| 1416619_at | 4632428N05Rik | RIKEN cDNA | BC003967 | 0 | Mm.273584 | 74048 |
| | | 4632428N05 gene | | | | |
| 1417898_a_at | Gzma | granzyme A | NM_010370 | P11032 | Mm.15510 | 14938 |
| 1449393_at | Sh2d1a | SH2 domain protein | NM_011364 | O88890 | Mm.235391 | 20400 |
| | | 1A | | | | |
| 1438577_at | 0 | Transcribed locus | BB376947 | 0 | Mm.130040 | 0 |
| 1416759_at | Mical1 | microtubule | NM_138315 | 0 | Mm.290431 | 171580 |
| | | associated | | | | |
| | | monoxygenase, | | | | |
| | | calponin and LIM | | | | |
| | | domain containing 1 | | | | |
| 1436905_x_at | Laptm5 | lysosomal-associated | BB218107 | Q61168, | Mm.271868 | 16792 |
| | | protein | | Q60924 | | |
| | | transmembrane 5 | | | | |
| 1418396_at | Gpsm3 | G-protein signalling | NM_134116 | 0 | Mm.26584 | 106512 |
| | | modulator 3 (AGS3- | | | | |
| | | like, C. elegans) | | | | |
| 1424724_a_at | D16Ertd472e | DNA segment, Chr | BC019957 | 0 | Mm.37332 | 67102 |
| | | 16, ERATO Doi 472, | | | | |
| | | expressed | | | | |
| 1429947_a_at | Zbp1 | Z-DNA binding | AK008179 | 0 | Mm.116687 | 58203 |
| | | protein 1 | | | | |
| 1448748_at | Plek | pleckstrin | AF181829 | 0 | Mm.98232 | 56193 |
| 1417620_at | Rac2 | RAS-related C3 | NM_009008 | Q05144 | Mm.1972 | 19354 |
| | | botulinum substrate 2 | | | | |
| 1427911_at | 2610307O08Rik | RIKEN cDNA | AK012006 | 0 | Mm.45995 | 72512 |
| | | 2610307O08 gene | | | | |
| 1451154_a_at | Cugbp2 | CUG triplet repeat, | BB644164 | 0 | Mm.147091 | 14007 |
| | | RNA binding protein 2 | | | | |
| 1416008_at | Satb1 | special AT-rich | AV172776 | Q60611 | Mm.311655 | 20230 |
| | | sequence binding | | | | |
| | | protein 1 | | | | |
| 1442700_at | Pde4b | phosphodiesterase | BG793493 | 0 | Mm.20181 | 18578 |
| | | 4B, cAMP specific | | | | |
| 1437249_at | Scap1 | src family associated | BG075562 | 0 | Mm.340720 | 78473 |
| | | phosphoprotein 1 | | | | |
| 1438475_at | 0 | 0 | BM246462 | 0 | 0 | 0 |
| 1421931_at | Icos | inducible T-cell co- | AB023132 | 0 | Mm.42044 | 54167 |
| | | stimulator | | | | |
| 1419206_at | Cd37 | CD37 antigen | BC019402 | Q61470 | Mm.3689 | 12493 |
| 1449175_at | Gpr65 | G-protein coupled | NM_008152 | Q61038 | Mm.207528 | 14744 |
| | | receptor 65 | | | | |
| 1422701_at | Zap70 | zeta-chain (TCR) | NM_009539 | P43404, | Mm.8038 | 22637 |
| | | associated protein | | P97455 | | |
| | | kinase | | | | |
| 1450291_s_at | Ms4a4c | membrane-spanning | NM_022429 | 0 | Mm.353643 | 64380 |
| | | 4-domains, subfamily | | | | |
| | | A, member 4C | | | | |
| 1417601_at | Rgs1 | regulator of G-protein | NM_015811 | 0 | Mm.103701 | 50778 |
| | | signaling 1 | | | | |
| 1437072_at | Arhgap25 | Rho GTPase | BM241218 | 0 | Mm.119564 | 232201 |
| | | activating protein 25 | | | | |
| 1436847_s_at | Cdca8 | cell division cycle | BB702047 | 0 | Mm.28038 | 52276 |
| | | associated 8 | | | | |
| 1457404_at | Nfkbiz | nuclear factor of | BM240058 | 0 | Mm.247272 | 80859 |
| | | kappa light | | | | |
| | | polypeptide gene | | | | |
| | | enhancer in B-cells | | | | |
| | | inhibitor, zeta | | | | |
| 1421173_at | Irf4 | interferon regulatory | U34307 | Q64287 | Mm.4677 | 16364 |
| | | factor 4 | | | | |
| 1416295_a_at | Il2rg | interleukin 2 | L20048 | P34902 | Mm.2923 | 16186 |
| | | receptor, gamma | | | | |
| | | chain | | | | |
| 1428242_at | 6330406L22Rik | RIKEN cDNA | AK018130 | 0 | Mm.243954 | 70719 |
| | | 6330406L22 gene | | | | |
| 1418392_a_at | Gbp4 | guanylate nucleotide | NM_018734 | Q61107 | Mm.1909 | 55932 |
| | | binding protein 4 | | | | |
| 1437025_at | Cd28 | CD28 antigen | AV313615 | P31041 | Mm.255003 | 12487 |
| 1422637_at | Rassf5 | Ras association | NM_018750 | O70407 | Mm.248291 | 54354 |
| | | (RalGDS/AF-6) | | | | |
| | | domain family 5 | | | | |
| 1439323_a_at | Map4k1 | mitogen activated | BB546619 | P70218 | Mm.148278 | 26411 |
| | | protein kinase kinase | | | | |
| | | kinase kinase 1 | | | | |
| 1424674_at | Slc39a6 | solute carrier family | BB825002 | 0 | Mm.21688 | 106957 |
| | | 39 (metal ion | | | | |
| | | transporter), member 6 | | | | |
| 1434920_a_at | Evl | Ena-vasodilator | AW553781 | P70429 | Mm.238841 | 14026 |
| | | stimulated | | | | |
| | | phosphoprotein | | | | |
| 1415850_at | Rasa3 | RAS p21 protein | NM_009025 | Q60790 | Mm.18517 | 19414 |
| | | activator 3 | | | | |
| 1435560_at | 0 | 0 | BI554446 | 0 | 0 | 0 |
| 1428735_at | Cd69 | CD69 antigen | AK017979 | 0 | Mm.74745 | 12515 |
| 1434573_at | Traf3ip3 | TRAF3 interacting | BE986588 | 0 | Mm.261259 | 215243 |
| | | protein 3 | | | | |
| 1419060_at | Gzmb | granzyme B | NM_013542 | P04187 | Mm.14874 | 14939 |
| 1450241_a_at | Evi2a | ecotropic viral | NM_010161 | P20934 | Mm.164948 | 14017 |
| | | integration site 2a | | | | |
| 1442219_at | Ms4a6b | Membrane-spanning | BB218965 | 0 | Mm.278844 | 69774 |
| | | 4-domains, subfamily | | | | |
| | | A, member 6B | | | | |
| 1460337_at | Sh3kbp1 | SH3-domain kinase | BB326929 | 0 | Mm.286495 | 58194 |
| | | binding protein 1 | | | | |
| 1425084_at | Gimap7 | GTPase, IMAP | BC026200 | 0 | Mm.30479 | 231932 |
| | | family member 7 | | | | |
| 1435343_at | Dock10 | dedicator of | BF715043 | 0 | Mm.133473 | 210293 |
| | | cytokinesis 10 | | | | |
| 1436598_at | Icos | inducible T-cell co- | AV313923 | 0 | Mm.42044 | 54167 |
| | | stimulator | | | | |
| 1422612_at | Hk2 | hexokinase 2 | NM_013820 | O08528 | Mm.255848 | 15277 |
| 1423135_at | Thy1 | thymus cell antigen 1, | AV028402 | P01831 | Mm.3951 | 21838 |
| | | theta | | | | |
| 1439436_x_at | Incenp | inner centromere | BB418702 | 0 | Mm.29755 | 16319 |
| | | protein | | | | |
| 1426505_at | Evi2b | ecotropic viral | AI122415 | 0 | 0 | 216984 |
| | | integration site 2b | | | | |
| 1420515_a_at | Pglyrp2 | peptidoglycan | NM_021319 | 0 | Mm.86752 | 57757 |
| | | recognition protein 2 | | | | |
| 1448511_at | Ptprcap | protein tyrosine | NM_016933 | Q64697 | Mm.329686 | 19265 |
| | | phosphatase, receptor | | | | |
| | | type, C polypeptide- | | | | |
| | | associated protein | | | | |
| 1442338_at | 0 | Transcribed locus | BB740904 | 0 | Mm.35746 | 0 |
| 1417391_a_at | Il16 | interleukin 16 | BC026894 | O54824 | Mm.10137 | 16170 |
| 1434376_at | Cd44 | CD44 antigen | AW146109 | P15379 | Mm.330428 | 12505 |
| 1433465_a_at | AI467606 | expressed sequence | BB234337 | 0 | Mm.284102 | 101602 |
| | | AI467606 | | | | |
| 1460253_at | Cklfsf7 | chemokine-like factor | NM_133978 | 0 | Mm.35600 | 102545 |
| | | super family 7 | | | | |
| 1429028_at | Dock11 | dedicator of | AK017170 | 0 | Mm.32873 | 75974 |
| | | cytokinesis 11 | | | | |
| 1428787_at | Nckap11 | NCK associated | BM238906 | 0 | Mm.30805 | 105855 |
| | | protein 1 like | | | | |
| 1436576_at | A630077B13Rik | RIKEN cDNA | BB239429 | 0 | Mm.34479 | 215900 |
| | | A630077B13 gene | | | | |
| 1440481_at | 0 | 0 | BB229853 | 0 | 0 | 0 |
| 1418353_at | Cd5 | CD5 antigen | NM_007650 | P13379 | Mm.779 | 12507 |
| 1427301_at | Cd48 | CD48 antigen | BE634960 | P18181 | Mm.1738 | 12506 |
| 1417756_a_at | Lsp1 | lymphocyte specific 1 | NM_019391 | P19973 | Mm.234003 | 16985 |
| 1422812_at | Cxcr6 | chemokine (C—X—C | NM_030712 | 0 | Mm.124289 | 80901 |
| | | motif) receptor 6 | | | | |
| 1456307_s_at | Adcy7 | Adenylate cyclase 7 | BB746807 | P51829 | Mm.288206 | 11513 |
| 1418131_at | Samhd1 | SAM domain and HD | NM_018851 | Q60710 | Mm.248478 | 56045 |
| | | domain, 1 | | | | |
| 1455132_at | A430107D22Rik | RIKEN cDNA | AV312663 | 0 | Mm.122284 | 320484 |
| | | A430107D22 gene | | | | |
| 1440275_at | Runx3 | Runt related | AV233043 | Q64131, | Mm.247493 | 12399 |
| | | transcription factor 3 | | O88674 | | |
| 1417786_a_at | Rgs19 | regulator of G-protein | BC003838 | 0 | Mm.274366 | 56470 |
| | | signaling 19 | | | | |
| 1448449_at | Ripk3 | receptor-interacting | NM_019955 | 0 | Mm.46612 | 56532 |
| | | serine-threonine | | | | |
| | | kinase 3 | | | | |
| 1422632_at | Ctsw | cathepsin W | NM_009985 | P56203 | Mm.113590 | 13041 |
| 1454694_a_at | Top2a | topoisomerase | BM211413 | Q01320 | Mm.4237 | 21973 |
| | | (DNA) II alpha | | | | |
| 1434940_x_at | Rgs19 | regulator of G-protein | BB233670 | 0 | Mm.274366 | 56470 |
| | | signaling 19 | | | | |
| 1449156_at | Ly9 | lymphocyte antigen 9 | NM_008534 | Q01965 | Mm.560 | 17085 |
| 1435084_at | C730049O14Rik | RIKEN cDNA | BB200607 | 0 | Mm.209644 | 320117 |
| | | C730049O14 gene | | | | |
| 1420819_at | Sla | src-like adaptor | NM_009192 | Q60898 | Mm.7601 | 20491 |
| 1434067_at | AI662270 | expressed sequence | BE688410 | 0 | Mm.295569 | 103814 |
| | | AI662270 | | | | |
| 1416007_at | Satb1 | special AT-rich | AV172776 | Q60611 | Mm.311655 | 20230 |
| | | sequence binding | | | | |
| | | protein 1 | | | | |
| 1452087_at | Epsti1 | epithelial stromal | BF020640 | 0 | Mm.68134 | 108670 |
| | | interaction 1 (breast) | | | | |
| 1436649_at | Zfpn1a3 | RIKEN cDNA | BB151746 | O08900 | Mm.133367 | 22780 |
| | | 5830411O07 gene | | | | |
| 1449235_at | Fasl | Fas ligand (TNF | NM_010177 | P41047 | Mm.3355 | 14103 |
| | | superfamily, member | | | | |
| | | 6) | | | | |
| 1450639_at | Slc28a2 | solute carrier family | NM_021520 | O88627 | Mm.29510 | 269346, |
| | | 28 (sodium-coupled | | | | 381417 |
| | | nucleoside | | | | |
| | | transporter), member 2 | | | | |
| 1416076_at | Ccnb1-rs1 | cyclin B1, related | NM_007629 | P24860 | Mm.260114 | 12429, |
| | | sequence 1 | | | | 268697, |
| | | | | | | 434175, |
| | | | | | | 545021 |
| 1421038_a_at | Kcnn4 | potassium | NM_008433 | O89109 | Mm.9911 | 16534 |
| | | intermediate/small | | | | |
| | | conductance calcium- | | | | |
| | | activated channel, | | | | |
| | | subfamily N, member 4 | | | | |
| 1447792_x_at | 0 | Adult male thymus | BB241847 | 0 | Mm.179798 | 0 |
| | | cDNA, RIKEN full- | | | | |
| | | length enriched | | | | |
| | | library, | | | | |
| | | clone: 5830404C02 | | | | |
| | | product: unknown | | | | |
| | | EST, full insert | | | | |
| | | sequence | | | | |
| 1419598_at | Ms4a6d | membrane-spanning | NM_026835 | 0 | Mm.290390 | 68774 |
| | | 4-domains, subfamily | | | | |
| | | A, member 6D | | | | |
| 1426159_x_at | Tcrb-V13 | T-cell receptor beta, | U46841 | 0 | Mm.333026 | 269846 |
| | | variable 13 | | | | |
| 1456014_s_at | BC032204 | cDNA sequence | BB113173 | 0 | Mm.157591 | 108101 |
| | | BC032204 | | | | |
| 1443534_at | 0 | 0 | BM201095 | 0 | 0 | 0 |
| 1419226_at | Cd96 | CD96 antigen | NM_032465 | 0 | Mm.29204 | 84544 |
| 1428696_at | 2310015N21Rik | RIKEN cDNA | AK009372 | 0 | Mm.41854 | 76438 |
| | | 2310015N21 gene | | | | |
| 1448314_at | Cdc2a | cell division cycle 2 | NM_007659 | P11440 | Mm.281367 | 12534 |
| | | homolog A (S. pombe) | | | | |
| 1424443_at | Tm6sf1 | transmembrane 6 | AV378394 | P58749 | Mm.221412 | 107769 |
| | | superfamily member 1 | | | | |
| 1433826_at | AW212607 | expressed sequence | AV325152 | 0 | Mm.277243 | 241732 |
| | | AW212607 | | | | |
| 1455269_a_at | Corola | coronin, actin binding | BB740218 | O89053 | Mm.290482 | 12721 |
| | | protein 1A | | | | |
| 1450106_a_at | Evl | Ena-vasodilator | NM_007965 | P70429 | Mm.238841 | 14026 |
| | | stimulated | | | | |
| | | phosphoprotein | | | | |
| 1434399_at | Galnt6 | UDP-N-acetyl-alpha- | AV231866 | 0 | Mm.22969 | 207839 |
| | | D- | | | | |
| | | galactosamine: polypeptide | | | | |
| | | N- | | | | |
| | | acetylgalactosaminyltransferase 6 | | | | |
| 1419153_at | 2810417H13Rik | RIKEN cDNA | AK017673 | 0 | Mm.269025 | 68026 |
| | | 2810417H13 gene | | | | |
| 1426278_at | Ifi27 | interferon, alpha- | AY090098 | 0 | Mm.271275 | 76933 |
| | | inducible protein 27 | | | | |
| 1432459_a_at | MGI: 1891838 | repressor of GATA | AK015881 | 0 | Mm.116789 | 58206 |
| 1451860_a_at | Trim30 | tripartite motif | AF220015 | P15533 | Mm.295578 | 20128 |
| | | protein 30 | | | | |
| 1452393_at | AI597013 | expressed sequence | BB014626 | 0 | Mm.258930 | 100182 |
| | | AI597013 | | | | |
| 1452205_x_at | Tcrb-V13 | T-cell receptor beta, | X67128 | 0 | Mm.333026 | 269846 |
| | | variable 13 | | | | |
| 1420394_s_at | Gp49a | glycoprotein 49 A | U05264 | Q61450, | Mm.358601 | 14727, |
| | | | | Q64281 | | 14728 |
| 1427656_at | Tcrb-V13 | T-cell receptor beta, | X14388 | 0 | Mm.333026 | 269846 |
| | | variable 13 | | | | |
| 1430165_at | Stk17b | serine/threonine | AI661948 | 0 | Mm.25559 | 98267 |
| | | kinase 17b | | | | |
| | | (apoptosis-inducing) | | | | |
| 1450997_at | Stk17b | serine/threonine | AV173139 | 0 | Mm.25559 | 98267 |
| | | kinase 17b | | | | |
| | | (apoptosis-inducing) | | | | |
| 1415899_at | Junb | Jun-B oncogene | NM_008416 | P10922, | Mm.1167 | 16477 |
| | | | | P09450 | | |
| 1449988_at | Gimap1 | GTPase, IMAP | NM_008376 | P70224 | Mm.252599 | 16205 |
| | | family member 1 | | | | |
| 1431292_a_at | Ptk91 | protein tyrosine | AK002699 | 0 | Mm.274346 | 23999 |
| | | kinase 9-like (A6- | | | | |
| | | related protein) | | | | |
| 1447621_s_at | 2610307O08Rik | RIKEN cDNA | AV300716 | 0 | Mm.45995 | 72512 |
| | | 2610307O08 gene | | | | |
| 1434980_at | Pik3r5 | phosphoinositide-3- | AV230647 | 0 | Mm.244960 | 320207 |
| | | kinase, regulatory | | | | |
| | | subunit 5, p101 | | | | |
| 1424953_at | BC021614 | cDNA sequence | BC021614 | 0 | Mm.26996 | 225884 |
| | | BC021614 | | | | |
| 1435144_at | 0 | Transcribed locus | BM243379 | 0 | Mm.364092 | 0 |
| 1433963_a_at | BC032204 | cDNA sequence | BG066664 | 0 | Mm.157591 | 108101 |
| | | BC032204 | | | | |
| 1419599_s_at | Ms4a11 | membrane-spanning | NM_026835 | 0 | 0 | 64382 |
| | | 4-domains, subfamily | | | | |
| | | A, member 11 | | | | |
| 1422303_a_at | Tnfrsf18 | tumor necrosis factor | AF229434 | O35714 | Mm.3180 | 21936 |
| | | receptor superfamily, | | | | |
| | | member 18 | | | | |
| 1450678_at | Itgb2 | integrin beta 2 | NM_008404 | P11835 | Mm.1137 | 16414 |
| 1427892_at | Myolg | myosin IG | BB235320 | 0 | Mm.239554 | 246177 |
| 1427511_at | B2m | Beta-2 microglobulin | AA170322 | P01887 | Mm.163 | 12010 |
| 1444177_at | 0 | Transcribed locus, | AI451538 | 0 | Mm.31556 | 0 |
| | | moderately similar to | | | | |
| | | XP_576460.1 | | | | |
| | | PREDICTED: similar | | | | |
| | | to hypothetical | | | | |
| | | protein | | | | |
| | | PB402898.00.0 | | | | |
| | | [Rattus norvegicus] | | | | |
| 1452539_a_at | Cd3z | CD3 antigen, zeta | X84237 | P29020, | Mm.217308 | 12503 |
| | | polypeptide | | P24161 | | |
| 1416882_at | Rgs10 | regulator of G-protein | NM_026418 | 0 | Mm.18635 | 67865 |
| | | signalling 10 | | | | |
| 1449361_at | Tbx21 | T-box 21 | NM_019507 | 0 | Mm.94519 | 57765 |
| 1417065_at | Egr1 | early growth response 1 | NM_007913 | P08046 | Mm.181959 | 13653 |
| 1425860_x_at | Cklf | chemokine-like factor | AY046597 | 0 | Mm.269219 | 75458 |
| 1419561_at | Ccl3 | chemokine (C-C | NM_011337 | P10855 | Mm.1282 | 20302 |
| | | motif) ligand 3 | | | | |
| 1450753_at | Nkg7 | natural killer cell | NM_024253 | 0 | Mm.34613 | 72310 |
| | | group 7 sequence | | | | |
| 1422875_at | Cd84 | CD84 antigen | NM_013489 | 0 | Mm.259115 | 12523 |
| 1426817_at | Mki67 | antigen identified by | X82786 | Q61769 | Mm.4078 | 17345 |
| | | monoclonal antibody | | | | |
| | | Ki 67 | | | | |
| 1418655_at | Galgt1 | UDP-N-acetyl-alpha- | U18975 | Q09200 | Mm.1853 | 14421 |
| | | D-galactosamine: (N- | | | | |
| | | acetylneuraminyl)- | | | | |
| | | galactosylglucosylceramide- | | | | |
| | | beta-1,4-N- | | | | |
| | | acetylgalactosaminyltransferase | | | | |
| 1456439_x_at | Mical1 | microtubule | BB209438 | 0 | Mm.290431 | 171580 |
| | | associated | | | | |
| | | monoxygenase, | | | | |
| | | calponin and LIM | | | | |
| | | domain containing 1 | | | | |
| 1452348_s_at | Mnda | myeloid cell nuclear | AI481797 | 0 | 0 | 381308 |
| | | differentiation | | | | |
| | | antigen | | | | |
| 1453228_at | Stx11 | syntaxin 11 | AK017897 | 0 | Mm.248648 | 74732 |
| 1449347_a_at | Xlr4 | X-linked | NM_021365 | 0 | Mm.104764 | 27083, |
| | | lymphocyte-regulated 4 | | | | 434794 |
| 1416379_at | Panx1 | pannexin 1 | NM_019482 | 0 | Mm.142253 | 55991 |
| 1416935_at | Trpv2 | transient receptor | NM_011706 | 0 | Mm.288064 | 22368 |
| | | potential cation | | | | |
| | | channel, subfamily V, | | | | |
| | | member 2 | | | | |
| 1450069_a_at | Cugbp2 | CUG triplet repeat, | BB667096 | 0 | Mm.147091 | 14007 |
| | | RNA binding protein 2 | | | | |
| 1458299_s_at | Nfkbie | nuclear factor of | BB820441 | O54910 | Mm.57043 | 18037 |
| | | kappa light | | | | |
| | | polypeptide gene | | | | |
| | | enhancer in B-cells | | | | |
| | | inhibitor, epsilon | | | | |
| 1415945_at | Mcm5 | minichromosome | NM_008566 | P49718 | Mm.5048 | 17218 |
| | | maintenance deficient | | | | |
| | | 5, cell division cycle | | | | |
| | | 46 (S. cerevisiae) | | | | |
| 1426170_a_at | Cd8b1 | CD8 antigen, beta | U34882 | P10300 | Mm.333148 | 12526 |
| | | chain 1 | | | | |
| 1434388_at | Mobkl2a | MOB1, Mps One | BB023868 | 0 | Mm.49309 | 208228 |
| | | Binder kinase | | | | |
| | | activator-like 2A | | | | |
| | | (yeast) | | | | |
| 1428786_at | Nckap1l | NCK associated | BM238906 | 0 | Mm.30805 | 105855 |
| | | protein 1 like | | | | |
| 1429525_s_at | Myo1f | myosin IF | AK021181 | 0 | Mm.42019 | 17916 |
| 1419004_s_at | Bcl2a1a | B-cell | L16462 | Q07440, | Mm.244917 | 12044, |
| | | leukemia/lymphoma | | O55179 | | 12045, |
| | | 2 related protein A1a | | | | 12047 |
| 1421317_x_at | Myb | myeloblastosis | NM_033597 | P06876, | Mm.52109 | 17863 |
| | | oncogene | | Q61927, | | |
| | | | | Q61421, | | |
| | | | | Q61926, | | |
| | | | | Q61928 | | |
| 1443894_at | Evi2b | ecotropic viral | BB236216 | 0 | 0 | 216984 |
| | | integration site 2b | | | | |
| 1433699_at | Tnfaip3 | tumor necrosis factor, | BM241351 | Q60769 | Mm.116683 | 21929 |
| | | alpha-induced protein 3 | | | | |
| 1452389_at | Tnfrsf7 | tumor necrosis factor | L24495 | P41272 | Mm.121 | 21940 |
| | | receptor superfamily, | | | | |
| | | member 7 | | | | |
| 1418398_a_at | Phemx | pan hematopoietic | AF175771 | 0 | Mm.28172 | 27027 |
| | | expression | | | | |
| 1419186_a_at | St8sia4 | ST8 alpha-N-acetyl- | NM_009183 | Q64692 | Mm.306228 | 20452 |
| | | neuraminide alpha- | | | | |
| | | 2,8-sialyltransferase 4 | | | | |
| 1438676_at | Mpa2l | macrophage | BM241485 | 0 | Mm.275893 | 100702 |
| | | activation 2 like | | | | |
| 1423182_at | 0 | 0 | AK004668 | 0 | 0 | 0 |
| 1421628_at | Il18r1 | interleukin 18 | NM_008365 | Q61098 | Mm.253664 | 16182 |
| | | receptor 1 | | | | |
| 1424906_at | E030024M05Rik | RIKEN cDNA | BC025220 | 0 | Mm.5675 | 217430 |
| | | E030024M05 gene | | | | |
| 1418612_at | Slfn1 | schlafen 1 | NM_011407 | 0 | Mm.10948 | 20555 |
| 1418776_at | 5830443L24Rik | RIKEN cDNA | NM_029509 | 0 | Mm.301868 | 76074 |
| | | 5830443L24 gene | | | | |
| 1439440_x_at | Ptk9l | protein tyrosine | BB397672 | 0 | Mm.274346 | 23999 |
| | | kinase 9-like (A6- | | | | |
| | | related protein) | | | | |
| 1434068_s_at | AI662270 | expressed sequence | BE688410 | 0 | Mm.295569 | 103814 |
| | | AI662270 | | | | |
| 1435458_at | 0 | 0 | AI323550 | 0 | 0 | 0 |
| 1453281_at | Pik3cd | Phosphatidylinositol | BB700084 | O35904 | Mm.229108 | 18707 |
| | | 3-kinase catalytic | | | | |
| | | delta polypeptide | | | | |
| 1435710_at | AI661384 | expressed sequence | BB034038 | 0 | Mm.30743 | 106930 |
| | | AI661384 | | | | |
| 1451673_at | Cd8a | CD8 antigen, alpha | M12825 | P01731, | Mm.1858 | 12525 |
| | | chain | | Q60965 | | |
| 1452815_at | P2ry10 | purinergic receptor | AK020001 | 0 | Mm.74639 | 78826 |
| | | P2Y, G-protein | | | | |
| | | coupled 10 | | | | |
| 1416811_s_at | Ctla2a | cytotoxic T | NM_007796 | P12399, | Mm.358584 | 13024, |
| | | lymphocyte- | | P12400 | | 13025 |
| | | associated protein 2 | | | | |
| | | alpha | | | | |
| 1436329_at | Egr3 | early growth response 3 | AV346607 | P43300 | Mm.103737 | 13655 |
| 1416875_at | Parvg | parvin, gamma | NM_022321 | 0 | Mm.251356 | 64099 |
| 1423467_at | Ms4a4b | membrane-spanning | BB199001 | 0 | Mm.33957 | 60361 |
| | | 4-domains, subfamily | | | | |
| | | A, member 4B | | | | |
| 1444078_at | Cd8a | CD8 antigen, alpha | BB154331 | P01731, | Mm.1858 | 12525 |
| | | chain | | Q60965 | | |
| 1436808_x_at | Mcm5 | minichromosome | AI324988 | P49718 | Mm.5048 | 17218 |
| | | maintenance deficient | | | | |
| | | 5, cell division cycle | | | | |
| | | 46 (S. cerevisiae) | | | | |
| 1416802_a_at | Cdca5 | cell division cycle | NM_026410 | 0 | Mm.23526 | 67849 |
| | | associated 5 | | | | |
| 1426239_s_at | 0 | 0 | BC016642 | 0 | 0 | 0 |
| 1416028_a_at | Hn1 | hematological and | NM_008258 | P97825 | Mm.1775 | 15374 |
| | | neurological | | | | |
| | | expressed sequence 1 | | | | |
| 1429524_at | Myo1f | myosin IF | AK021181 | 0 | Mm.42019 | 17916 |
| 1419254_at | Mthfd2 | methylenetetrahydrofolate | BG076333 | P18155 | Mm.443 | 17768 |
| | | dehydrogenase | | | | |
| | | (NAD+ dependent), | | | | |
| | | methenyltetrahydrofolate | | | | |
| | | cyclohydrolase | | | | |
| 1441317_x_at | MGI: 1923321 | gamma-aminobutyric | BB316060 | 0 | Mm.228812 | 76071 |
| | | acid (GABA-B) | | | | |
| | | receptor binding | | | | |
| | | protein | | | | |
| 1438917_x_at | Nup62 | nucleoporin 62 | AW240611 | Q63850 | Mm.2565 | 18226 |
| 1429319_at | Rhoh | ras homolog gene | BM243660 | 0 | Mm.358763 | 74734 |
| | | family, member H | | | | |
| 1437636_at | LOC433377 | similar to Interferon- | BB135602 | 0 | 0 | 433377 |
| | | activatable protein | | | | |
| | | 203 (Ifi-203) | | | | |
| | | (Interferon-inducible | | | | |
| | | protein p203) | | | | |
| 1435330_at | AI447904 | expressed sequence | BM241008 | 0 | Mm.360525 | 236312, |
| | | AI447904 | | | | 545384 |
| 1416698_a_at | Cks1b | CDC28 protein | NM_016904 | P61025 | Mm.3049 | 54124 |
| | | kinase 1b | | | | |
| 1460651_at | Lat | linker for activation | AF036907 | O54957 | Mm.10280 | 16797 |
| | | of T cells | | | | |
| 1433964_s_at | BC032204 | cDNA sequence | BG066664 | 0 | Mm.157591 | 108101 |
| | | BC032204 | | | | |
| 1434295_at | Rasgrp1 | RAS guanyl releasing | BE691356 | 0 | Mm.42150 | 19419 |
| | | protein 1 | | | | |
| 1437325_x_at | Aldh18a1 | aldehyde | BB251523 | Q63739 | Mm.233117 | 56454 |
| | | dehydrogenase 18 | | | | |
| | | family, member A1 | | | | |
| 1426772_x_at | Tcrb-J | T-cell receptor beta, | M11456 | 0 | Mm.333026 | 21580, |
| | | joining region | | | | 269846, |
| | | | | | | 381765 |
| 1451363_a_at | 2010308M01Rik | RIKEN cDNA | BC008266 | 0 | Mm.371646 | 72121 |
| | | 2010308M01 gene | | | | |
| 1439814_at | 0 | Transcribed locus | BM246630 | 0 | Mm.315271 | 0 |
| 1448575_at | Il7r | interleukin 7 receptor | AI573431 | P16872 | Mm.389 | 16197 |
| 1422188_s_at | Tcrg | T-cell receptor | NM_011558 | 0 | Mm.350873 | 110067, |
| | | gamma chain | | | | 434531 |
| 1437760_at | Galnt12 | UDP-N-acetyl-alpha- | AV376137 | 0 | Mm.132246 | 230145 |
| | | D- | | | | |
| | | galactosamine: polypeptide | | | | |
| | | N- | | | | |
| | | acetylgalactosaminyltransferase | | | | |
| | | 12 | | | | |
| 1428492_at | Glipr2 | GLI pathogenesis- | BM208214 | 0 | Mm.22213 | 384009 |
| | | related 2 | | | | |
| 1460437_at | Pscd4 | pleckstrin homology, | AK010908 | 0 | Mm.32911 | 72318 |
| | | Sec7 and coiled/coil | | | | |
| | | domains 4 | | | | |
| 1437052_s_at | Slc2a3 | solute carrier family | BB414515 | P32037, | Mm.269857 | 20527 |
| | | 2 (facilitated glucose | | Q61607 | | |
| | | transporter), member 3 | | | | |
| 1422638_s_at | Rassf5 | Ras association | NM_018750 | O70407 | Mm.248291 | 54354 |
| | | (RalGDS/AF-6) | | | | |
| | | domain family 5 | | | | |
| 1418826_at | Ms4a6b | membrane-spanning | NM_027209 | 0 | Mm.278844 | 69774 |
| | | 4-domains, subfamily | | | | |
| | | A, member 6B | | | | |
| 1422828_at | Cd3d | CD3 antigen, delta | NM_013487 | 0 | Mm.4527 | 12500 |
| | | polypeptide | | | | |
| 1452948_at | Tnfaip8l2 | tumor necrosis factor, | AK007540 | 0 | Mm.34368 | 69769 |
| | | alpha-induced protein | | | | |
| | | 8-like 2 | | | | |
| 1422932_a_at | Vav1 | vav 1 oncogene | NM_011691 | P27870, | Mm.248172 | 22324 |
| | | | | O08526 | | |
| 1436312_at | Zfpn1a1 | zinc finger protein, | AV317621 | Q03267 | Mm.103545 | 22778 |
| | | subfamily 1A, 1 | | | | |
| | | (Ikaros) | | | | |
| 1418451_at | Gng2 | guanine nucleotide | BB522409 | P63213 | Mm.41737 | 14702 |
| | | binding protein (G | | | | |
| | | protein), gamma 2 | | | | |
| | | subunit | | | | |
| 1418166_at | I112rb1 | interleukin 12 | NM_008353 | Q60837 | Mm.731 | 16161 |
| | | receptor, beta 1 | | | | |
| 1448749_at | Plek | pleckstrin | AF181829 | 0 | Mm.98232 | 56193 |
| 1452483_a_at | Cd44 | CD44 antigen | X66083 | P15379 | Mm.330428 | 12505 |
| 1448617_at | Cd53 | CD53 antigen | NM_007651 | Q61451 | Mm.316861 | 12508 |
| 1425832_a_at | Cxcr6 | chemokine (C—X—C | AF301018 | 0 | Mm.124289 | 80901 |
| | | motif) receptor 6 | | | | |
| 1421855_at | Fgl2 | fibrinogen-like | BF136544 | P12804 | Mm.292100 | 14190 |
| | | protein 2 | | | | |
| 1419202_at | Cst7 | cystatin F | NM_009977 | O89098 | Mm.12965 | 13011 |
| | | (leukocystatin) | | | | |
| 1423602_at | Traf1 | Tnf receptor- | BG064103 | P39428 | Mm.239514 | 22029 |
| | | associated factor 1 | | | | |
| 1450905_at | Plxnc1 | plexin C1 | BB476707 | 0 | Mm.256712 | 54712 |
| 1439141_at | Gpr18 | G protein-coupled | BG145550 | 0 | Mm.37405 | 110168 |
| | | receptor 18 | | | | |
| 1426324_at | H2-D1 | histocompatibility 2, | M33151 | P01899, | Mm.33263 | 14964 |
| | | D region locus 1 | | P01900, | | |
| | | | | P01897, | | |
| | | | | P01895, | | |
| | | | | Q31116, | | |
| | | | | Q31198, | | |
| | | | | Q31168, | | |
| | | | | O19467, | | |
| | | | | O78207, | | |
| | | | | Q31167, | | |
| | | | | Q31209, | | |
| | | | | Q31149, | | |
| | | | | Q31169, | | |
| | | | | Q31170, | | |
| | | | | Q31188, | | |
| | | | | Q61891, | | |
| | | | | Q61892 | | |
| 1425086_a_at | Slamf6 | SLAM family | AF248636 | 0 | Mm.245727 | 30925 |
| | | member 6 | | | | |
| 1420671_x_at | Ms4a4c | membrane-spanning | NM_029499 | 0 | Mm.353643 | 64380 |
| | | 4-domains, subfamily | | | | |
| | | A, member 4C | | | | |
| 1422628_at | 4632417K18Rik | RIKEN cDNA | NM_026640 | 0 | Mm.1643 | 107373 |
| | | 4632417K18 gene | | | | |
| 1417164_at | Dusp10 | dual specificity | NM_022019 | 0 | Mm.266191 | 63953 |
| | | phosphatase 10 | | | | |
| 1452796_at | Def6 | differentially | AK010356 | 0 | Mm.204731 | 23853 |
| | | expressed in FDCP 6 | | | | |
| 1419631_at | Was | Wiskott-Aldrich | NM_009515 | P70315, | Mm.4735 | 22376 |
| | | syndrome homolog | | Q61078 | | |
| | | (human) | | | | |
| 1421457_a_at | Samsn1 | SAM domain, SH3 | NM_023380 | P57725 | Mm.131406 | 67742 |
| | | domain and nuclear | | | | |
| | | localisation signals, 1 | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting tissue rejection, wherein said method comprises determining whether or not tissue transplanted into a mammal contains cells that express at least two of the nucleic acids listed in Table 4 or Table 5, wherein the presence of said cells indicates that said tissue is being rejected.

2. The method of claim 1, wherein said mammal is a human.

3. The method of claim 1, wherein said tissue is kidney tissue.

4. The method of claim 1, wherein said tissue is a kidney.

5. The method of claim 1, wherein said method comprises determining whether or not said tissue contains cells that express at least five of said nucleic acids.

6. The method of claim 1, wherein said method comprises determining whether or not said tissue contains cells that express at least ten of said nucleic acids.

7. The method of claim 1, wherein said method comprises determining whether or not said tissue contains cells that express at least twenty of said nucleic acids.

8. The method of claim 1, wherein said determining step comprises measuring the level of mRNA expressed from said at least two nucleic acids.

9. The method of claim 1, wherein said determining step comprises measuring the level of polypeptide expressed from said at least two nucleic acids.

10. The method of claim 1, wherein said method comprises determining whether or not said tissue contains cells that express at least two of said nucleic acids at a level greater than the average level of expression exhibited in cells from control tissue that has not been transplanted.
